(19) United States
(12) Patent Application Publication
(10) Pub. No.: US 2010/0317848 A1
(43) Pub. Date: Dec. 16, 2010

(54) PROCESS FOR THE SYNTHESIS OF DERIVATIVES OF 3-AMINO-TETRAHYDROFURAN-3-CARBOXYLIC ACID AND USE THEREOF AS MEDICAMENTS

(75) Inventors: Zhengxu Han, Shrewsbury, MA (US); Kai Gerlach, Mittelbiberach (DE); Dhileepkumar Krishnamurthy, Brookfield, CT (US); Burkhard Matthes, Bad Wurzach (DE); Herbert Nar, Ochsenhausen (DE); Roland Pfau, Biberach (DE); Henning Priepke, Warthausen (DE); Annette Schuler-Metz, Ulm (DE); Chris H. Senanayake, Brookfield, CT (US); Peter Sieger, Mittelbiberach (DE); Wenjun Tang, Southbury, CT (US); Wolfgang Wienen, Biberach (DE); Yibo Xu, New Milford, CT (US); Nathan K. Yee, Danbury, CT (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/521,608

(22) PCT Filed: Dec. 21, 2007
(86) PCT No.: PCT/EP07/64406

§ 371 Date: Oct. 8, 2009

(30) Foreign Application Priority Data
Dec. 31, 2006 (US) 60882937

(51) Int. Cl. C07D 413/14; C07D 409/14

(52) U.S. Cl. 540/488; 540/594; 540/544; 544/146; 548/527; 540/524; 540/586

(57) ABSTRACT

The present invention relates to a process for the manufacturing of substituted 3-amino-tetrahydrofuran-3-carboxylic acid amides of general formula (I) and their precursors in high optical purity to the precursors of the synthesis of substituted S-Amino-tetrahydrofuran-S-carboxylic acid amides of general formula (I) in high optical purity, and to the tautomers, enantiomers, diastereomers, mixtures and salts of substituted 3-amino-tetrahydrofuran-3-carboxylic acid amides of general formula (I) in high optical purity, particularly the physiologically acceptable salts thereof with inorganic or organic acids or bases, which have valuable properties.

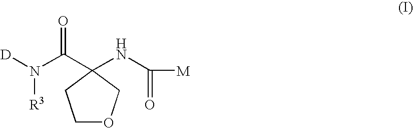

### BACKGROUND AND SUMMARY OF THE INVENTION

The present invention relates to a process for the manufacturing of substituted 3-amino-tetrahydrofuran-3-carboxylic acid amides of general formula (I) and their precursors in high optical purity

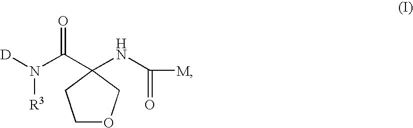

to the precursors of the synthesis of substituted 3-amino-tetrahydrofuran-3-carboxylic acid amides of general formula (I) in high optical purity, and to the tautomers, enantiomers, diastereomers, mixtures and salts of substituted 3-amino-tetrahydrofuran-3-carboxylic acid amides of general formula (I) in high optical purity, particularly the physiologically acceptable salts thereof with inorganic or organic acids or bases, which have valuable properties.

Thus, the present invention relates to the stereoselective preparation of compounds of the above general formula (I).

Within the meaning of the present invention, the high optical purity of the substituted 3-amino-tetrahydrofuran-3-carboxylic acid amides of general formula (I) and of the precursors of these substituted 3-amino-tetrahydrofuran-3-carboxylic acid amides of general formula (I) is with respect to the carbon atom in position 3 of the tetrahydrofuran ring, the position of which is depicted by the numbering “3” in the following structural formula (I)

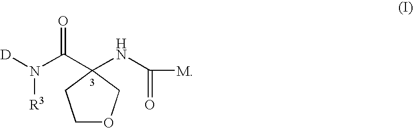

Within the meaning of the present invention, “highly optical pure” means in an enantiomeric excess of more than 96%, preferably of more than 98%.

The invention also relates to pharmaceutical compositions containing a compound or a physiologically acceptable salt of a compound of the above general formula (I) according to the embodiments defined below and in the Examples, optionally together with one or more inert carriers and/or diluents.

The invention also relates to the use of a compound or a physiologically acceptable salt of a compound according to the embodiments defined below and in the Examples, for preparing a pharmaceutical composition with an inhibitory effect on Factor Xa, an inhibitory effect on related serine proteases, and/or an antithrombotic activity.

Although the pharmacologically valuable properties of the compounds in accordance with the present invention constitute the basic prerequisite for effective use of the compounds in a pharmaceutical composition, an active substance must in any case satisfy additional requirements in order to be accepted for use as a drug. These parameters are largely connected with the physicochemical nature of the active substance. Hence, there continues to be a need for crystalline forms of active substances, which can be conveniently formulated for administration to patients and which are pure and highly crystalline in order to fulfil exact pharmaceutical requirements and specifications.

Preferably, such compounds will be readily formed and have favourable bulk characteristics. Examples of favourable bulk characteristics are drying times, filterability, solubility, intrinsic dissolution rate and stability in general.

As the crystal modification of an active substance is important to the reproducible active substance content of a preparation, there is a need to clarify as far as possible any existing polymorphism of an active substance present in crystalline form. If there are different polymorphic modifications of an active substance care must be taken to ensure that the crystalline modification of the substance does not change in the pharmaceutical preparation later produced from it. Otherwise, this could have a harmful effect on the reproducible potency of the drug. Against this background, active substances characterised by only slight polymorphism are preferred.

Decreased levels of organic solvents in the crystal lattice are also favourable, due in part to potential solvent toxicity to the recipient as a function of the solvent.

Another criterion which may be of exceptional importance under certain circumstances depending on the choice of formulation or the choice of manufacturing process is the solubility and dissolution rate of the active substance. If for example pharmaceutical solutions are prepared (e.g. for infusions) it is essential that the active substance should be sufficiently soluble in physiologically acceptable solvents. For drugs which are to be taken orally, it is in general very important that the active substance should be sufficiently soluble over a suitable range of pH, and bioavailable.

Hence, without being restrictive, examples of the parameters which need to be controlled are the stability of the starting substance under various environmental conditions, the stability during production of the pharmaceutical formulation and the stability in the final compositions of the drug.

The pharmaceutically active substance used to prepare the pharmaceutical compositions should therefore have great stability which is ensured even under all kinds of environmental conditions.

The present invention further provides a pharmaceutically active substance which is not only characterised by high pharmacological potency but also satisfies the physicochemical requirements of high purity and high crystallinity mentioned hereinbefore, in order to fulfil as far as possible exact pharmaceutical requirements and specifications.

Hence, the present invention relates as well to an anhydrous crystalline form of a compound which may be obtained via the manufacturing process in accordance with the present invention, namely the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide, to a process for the manufacture thereof, and to the use thereof in a pharmaceutical composition. The structure of this compound is depicted below in the form of the free base as example 2. The characterizing data of its anhydrous crystalline form are described further below in the experimental section.

The present invention also provides a process for the manufacture of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide, which is described further below in the experimental section.

### BRIEF DESCRIPTION OF THE FIGURES

FIG. 1 shows the X-ray powder diffractogram of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)tetrahydrofuran-3-carboxylic acid amide, recorded using a STOE Stadi P-diffractometer fitted with a location-sensitive detector (OED) and a Cu anode as the x-ray source and a Germanium monochromator (CuK radiation, =1.54056 Å, 40 kV, 40 mA).

FIG. 2 shows a light microscopy photograph of crystals of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide.

FIG. 3 shows the thermoanalysis and determination of the melting point and loss on drying (DSC/TG) of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide, recorded using a DSC and TG and evaluated by the peak onset (heating rate: 10° C./min) for the melting point and by the weight loss step between room temperature and 180° C. for the loss on drying. The values given are determined using a DSC 821e and a TGA/STDA 851e, both made by Mettler Toledo.

### DETAILED DESCRIPTION OF THE INVENTION

A 1st embodiment of the present invention includes those compounds of general formula (I) wherein

D denotes D¹ a substituted bicyclic ring system of formula (II),

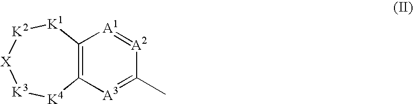

wherein

### K¹ and K⁴

each independently of one another denote a —CH₂, —CHR^{7a}, —CR^{7b}R^{7c} or a —C(O) group, and

### R^{7a}/R^{7b}/R^{7c}

each independently of one another denote a fluorine atom, a hydroxy, C₁₋₅-alkyloxy, amino, C₁₋₅-alkylamino, di-(C₁₋₅-alkyl)-amino, C₃₋₅-cycloalkyleneimino or C₁₋₅-alkylcarbonylamino group,
a C₁₋₅-alkyl group which may be substituted by 1-3 fluorine atoms, or
two groups R^{7b}/R^{7c} together with the cyclic carbon atom may form a 3, 4, 5-, 6- or 7-membered saturated carbocyclic group
wherein the methylene groups thereof may be substituted by 1-2 C₁₋₃-alkyl or CF₃— groups, and/or
the methylene groups thereof, if they are not bound to a heteroatom, may be substituted by 1-2 fluorine atoms, and

### K² and K³

each independently of one another denote a —CH₂, —CHR^{8a}, —CR^{8b}R^{8c} or a —C(O)— group, and

### R^{8a}/R^{8b}/R^{8c}

each independently of one another denote a C₁₋₅-alkyl group which may be substituted by 1-3 fluorine atoms,
or two groups R^{8b}/R^{8c} together with the cyclic carbon atom may form a 3, 4, 5-, 6- or 7-membered saturated carbocyclic group, and
in all there should be no more than four groups selected from among R^{7a}, R^{7b}, R^{7c}, R^{8a}, R^{8b}, and R^{8c} in formula (II), and
X denotes a NR¹ group, wherein
R¹ denotes a hydrogen atom or a hydroxy, C₁₋₃-alkyloxy, amino, C₁₋₃-alkylamino, di-(C₁₋₃-alkyl)-amino, a C₁₋₅-alkyl, C₂₋₅-alkenyl-CH₂, C₂₋₅-alkynyl-CH₂ or C₃₋₆-cycloalkyl group,
wherein the methylene and methyl groups present in the above-mentioned groups
may additionally be substituted by a C₁₋₃-alkyl, carboxy, C₁₋₅-alkoxycarbonyl group or
by a hydroxy, C₁₋₅-alkyloxy, amino, C₁₋₅-alkylamino, C₁₋₅-dialkylamino or C₄₋₇-cycloalkyleneimino group,
provided that O—C—O or O—C—N or N—C—N-bonds are excluded and/or one to three hydrogen atoms may be replaced by fluorine atoms, provided that the methylene or methyl groups are not directly bound to a nitrogen atom, and wherein
A¹ denotes either N or CR¹⁰,
A² denotes either N or CR¹¹,
A³ denotes either N or CR¹²,
wherein R¹⁰, R¹¹ and R¹² each independently of one another represent
a hydrogen, fluorine, chlorine, bromine or iodine atom, or a C₁₋₅-alkyl, CF₃, C₂₋₅-alkenyl, C₂₋₅-alkynyl, a cyano, carboxy, C₁₋₅-alkyloxycarbonyl, hydroxy, C₁₋₃-alkyloxy, CF₃O, CHF₂O, CH₂FO, or
   D denotes D² a group of general formula

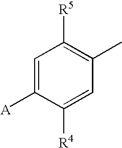

wherein A denotes A⁴, a group

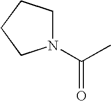

or wherein A denotes A⁵, a group of general formula

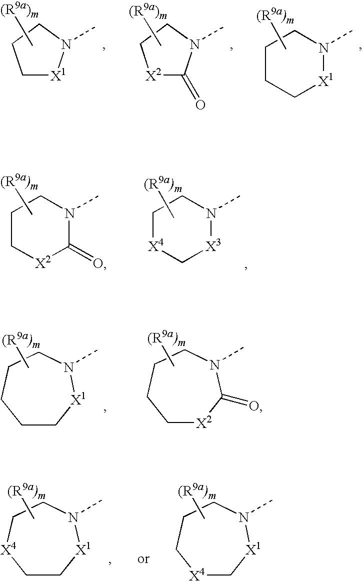

wherein
m is the number 1 or 2,
X¹ denotes a carbonyl, thiocarbonyl, C═NR^{9c}, C═N—OR^{9c}, C═N—NO₂, C═N—CN or sulphonyl group,
X² denotes an oxygen atom or a —NR^{9b} group,
X³ denotes a carbonyl, thiocarbonyl, C═NR^{9c}, C═N—OR^{9c}, C═N—NO₂, C═N—CN or sulphonyl group,
X⁴ denotes an oxygen or sulphur atom or a —NR^{9c} group,
R^{9a} in each case independently of one another denotes a hydrogen or halogen atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
in the previously mentioned substituted 5- to 7-membered groups A⁵ the heteroatoms F, Cl, Br, I, O or N optionally introduced with R^{9a} as substituent are not separated by precisely one carbon atom from a heteroatom selected from among N, O, S,
R^{9b} each independently of one another denote a hydrogen atom or a C₁₋₅-alkyl group,
R^{9b} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R⁴ denotes a hydrogen or halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R⁵ denotes a hydrogen or halogen atom or a C₁₋₃-alkyl group,
R³ denotes a hydrogen atom or a C₁₋₃-alkyl group, and
M denotes a thiophene ring according to formula (III),

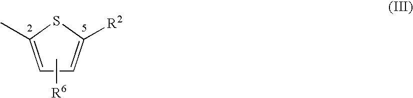

which is bound to the carbonyl group in formula (I) via the 2-position and which is substituted in the 5-position by R² and optionally additionally by R⁶, wherein
R² denotes
R^{2a} a hydrogen, fluorine or iodine atom, or
R^{2b} a methoxy, C₁₋₂-alkyl, formyl, NH₂CO, or
R^{2c} a chlorine, bromine atom or an ethynyl group,
R⁶ denotes a hydrogen, fluorine, chlorine, bromine or iodine atom or a C₁₋₂-alkyl or amino group,
wherein, unless otherwise stated, by the term “halogen atom” mentioned hereinbefore in the definitions is meant an atom selected from among fluorine, chlorine, bromine and iodine,
and wherein the alkyl, alkenyl, alkynyl and alkyloxy groups contained in the previously mentioned definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
the tautomers, enantiomers, diastereomers, mixtures and salts thereof.

Examples of the C₁₋₆-alkyl groups mentioned hereinbefore in the definitions are the methyl, ethyl, 1-propyl, 2-propyl, n-butyl, sec-butyl, tert-butyl, 1-pentyl, 2-pentyl, 3-pentyl, neo-pentyl, 3-methyl-2-butyl, 1-hexyl, 2-hexyl, 3-hexyl, 3-methyl-2-pentyl, 4-methyl-2-pentyl, 3-methyl-3-pentyl, 2-methyl-3-pentyl, 2,2-dimethyl-3-butyl or 2,3-dimethyl-2-butyl group.

Examples of the C₁₋₅-alkyloxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy, 3-pentyloxy or neo-pentyloxy group.

Examples of the C₂₋₅-alkenyl groups mentioned hereinbefore in the definitions are the ethenyl, 1-propen-1-yl, 2-propen-1-yl, 1-buten-1-yl, 2-buten-1-yl, 3-buten-1-yl, 1-penten-1-yl, 2-penten-1-yl, 3-penten-1-yl, 4-penten-1-yl, 1-hexen-1-yl, 2-hexen-1-yl, 3-hexen-1-yl, 4-hexen-1-yl, 5-hexen-1-yl, but-1-en-2-yl, but-2-en-2-yl, but-1-en-3-yl, 2-methyl-prop-2-en-1-yl, pent-1-en-2-yl, pent-2-en-2-yl, pent-3-en-2-yl, pent-4-en-2-yl, pent-1-en-3-yl, pent-2-en-3-yl, 2-methyl-but-1-en-1-yl, 2-methyl-but-2-en-1-yl, 2-methyl-but-3-en-1-yl or 2-ethyl-prop-2-en-1-yl group.

Examples of the C₂₋₅-alkynyl groups mentioned hereinbefore in the definitions are the ethynyl, 1-propynyl, 2-propynyl, 1-butyn-1-yl, 1-butyn-3-yl, 2-butyn-1-yl, 3-butyn-1-yl, 1-pentyn-1-yl, 1-pentyn-3-yl, 1-pentyn-4-yl, 2-pentyn-1-yl, 2-pentyn-3-yl, 3-pentyn-1-yl, 4-pentyn-1-yl, 2-methyl-1-butyn-4-yl, 3-methyl-1-butyn-1-yl or 3-methyl-1-butyn-3-yl group.

A 2nd embodiment of the present invention includes those compounds of general formula (I), wherein

D denotes a substituted bicyclic ring system of formula (II),

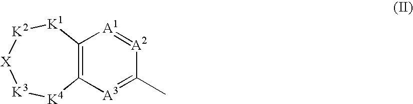

wherein

### K¹ and K⁴

each independently of one another denote a —CH₂, —CHR^{7a}, or a —CR^{7b}R^{7c} group, wherein

### R^{7a}/R^{7b}/R^{7c}

each independently of one another denote a fluorine atom, a hydroxy, methoxy or C₁₋₂-alkyl group which may be substituted by 1-3 fluorine atoms,
wherein the two groups R^{7b}/R^{7c} cannot both simultaneously be bound to the cyclic carbon atom via a heteroatom, except if —C(R^{7b}R^{7c})— corresponds to a —CF₂ group, or
two groups R^{7b}/R^{7c} may form, together with the cyclic carbon atom, a 3-, 4- or 5-membered saturated carbocyclic group, and

### K² and K³

each independently of one another represent a —CH₂, —CHR^{8a}, or —CR^{8b}R^{8c} group, and

### R^{8a}/R^{8b}/R^{8c}

each independently of one another denote a C₁₋₂-alkyl group which may be substituted by 1-3 fluorine atoms,
or two groups R^{8b}/R^{8c} may form, together with the cyclic carbon atom, a 3-, 4-, 5-membered carbocyclic group, and
in all in formula (II) there should be no more than four groups selected from among R^{7a}, R^{7b}, R^{7c}, R^{8a}, R^{8b} and R^{8c}, and
X denotes an NR¹ group, wherein
R¹ denotes a hydrogen atom or
a C₁₋₂-alkyl or C₃₋₄-cycloalkyl group,
wherein the methylene and methyl groups present in the above-mentioned groups may additionally be substituted by a methyl group,
and wherein
A¹ denotes CR¹⁰,
A² denotes CR¹¹,
A³ denotes CR¹²,
wherein R¹⁰, R¹¹ and R¹² each independently of one another represent
a hydrogen, fluorine, chlorine, bromine atom or a Methyl, CF₃, a cyano, Methoxy, CF₃O, CHF₂O, CH₂FO— group, or
D denotes D² a group of general formula

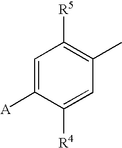

wherein A denotes A⁴, a group

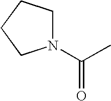

or wherein A denotes A⁵, a group of general formula

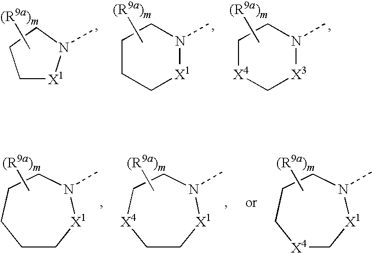

wherein
m is the number 1 or 2,
X¹ denotes a carbonyl or C═N—CN group,
X³ denotes a carbonyl or C═N—CN group,
X⁴ denotes an oxygen atom,
R^{9a} in each case independently of one another denotes a hydrogen atom or a C₁₋₂-alkyl group, while
R⁴ denotes a hydrogen or fluorine, chlorine or bromine atom, a methyl or a methoxy group,
R⁵ denotes a hydrogen, fluorine or chlorine atom or a methyl group,
R³ denotes a hydrogen atom and
M denotes a thiophene ring according to formula (III),

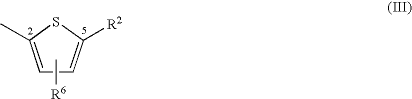

which is bound to the carbonyl group in formula (I) via the 2-position and which is substituted in the 5-position by R² and optionally additionally by R⁶, wherein
R² denotes
R^{2c} a chlorine, bromine atom or an ethynyl group,
R⁶ denotes a hydrogen atom,
the tautomers, diastereomers, mixtures and salts thereof.

A 3rd embodiment of the present invention includes all those compounds of the first and second embodiment, wherein

D denotes a substituted bicyclic ring system of formula (II),

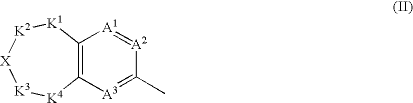

wherein K1, K2, K3, K4, X, A1, A2 and A3 are as defined in the 1^{st} or 2^{nd} embodiment,
the tautomers, diastereomers, mixtures and salts thereof.

A 4th embodiment of the present invention includes all those compounds of the first and second embodiment, wherein

D denotes D² a group of general formula

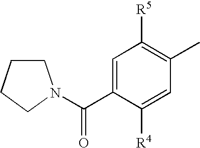

wherein R4 and R5 are as defined in the 1^{st} or 2^{nd} embodiment, the tautomers, diastereomers, mixtures and salts thereof.

A 5th embodiment of the present invention includes all those compounds of the first and second embodiment, wherein

D denotes D² a group of general formula

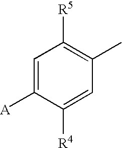

or wherein A denotes A⁵, wherein A5, R4 and R5 are as defined in the 1^{st} or 2^{nd} embodiment,
the tautomers, diastereomers, mixtures and salts thereof.

A 6th embodiment of the present invention includes all those compounds of the previous embodiments in high optical purity at the carbon in position 3 of the tetrahydrofuran ring, wherein the amino-tetrahydrofuran carboxylic acid amide moiety has the R-configuration.

A 7th embodiment of the present invention includes all those compounds of the previous embodiments in high optical purity at the carbon in position 3 of the tetrahydrofuran ring, wherein the amino-tetrahydrofuran carboxylic acid amide moiety has the S-configuration.

The following preferred compounds of general formula (I) are mentioned by way of example, both as the tautomers, mixtures and salts thereof:
(S)-3-[(5-bromo-thiophen-2-yl)-carbonylamino]-N-(3-methyl-2,3,4,5-tetrahydro-1H-benzo[d]azepin-7-yl)-tetrahydrofuran-3-carboxylic acid amide

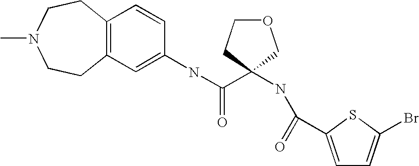

(S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

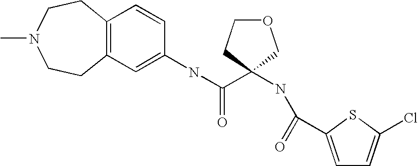

(3S)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide

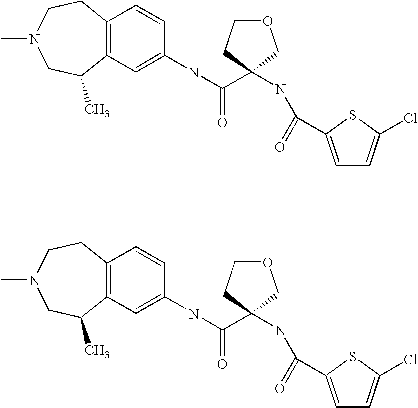

(3S)-3-[(5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amide

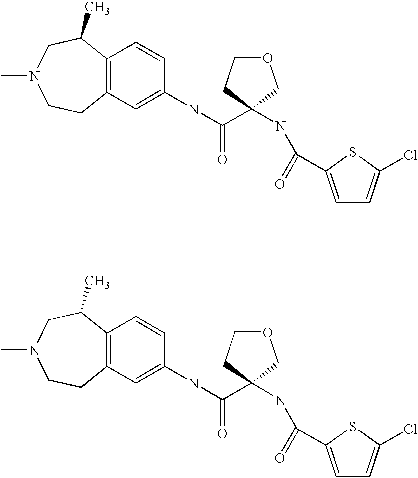

(S)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide

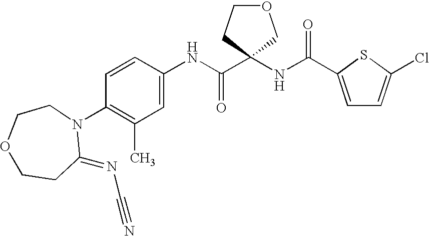

(S)-5-chloro-thiophene-2-carboxylic acid-N-{3-[4-(5-cyanimin-[1.4]oxazepan-4-yl)-phenylcarbamoyl]-tetrahydrofuran-3-yl}-amide

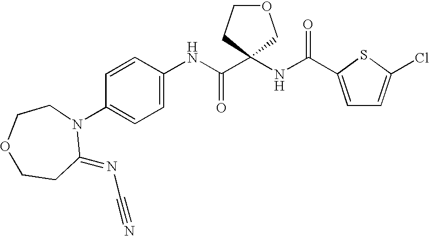

(S)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide

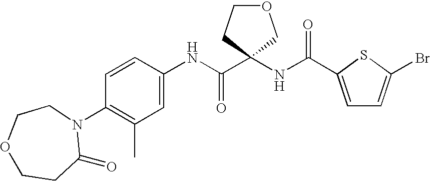

(S)-5-bromo-thiophene-2-carboxylic acid-N-{3-[4-(5-cyanimin-[1.4]oxazepan-4-yl)-phenylcarbamoyl]-tetrahydrofuran-3-yl}-amide

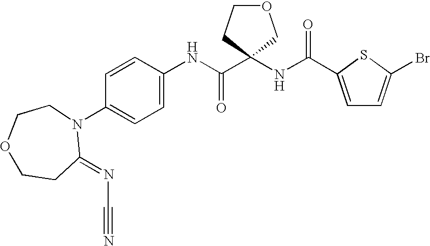

(S)-5-bromo-thiophene-2-carboxylic acid-N-{3-[3-methyl-4-(3-oxo-morpholin-4-yl)-phenylcarbamoyl]-tetrahydro-thiophen-3-yl}-amide

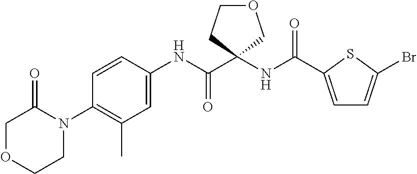

(S)-5-ethinyl-thiophene-2-carboxylic acid-N-{3-[4-(3-oxo-morpholin-4-yl)-phenylcarbamoyl]-tetrahydro-thiophen-3-yl}-amide

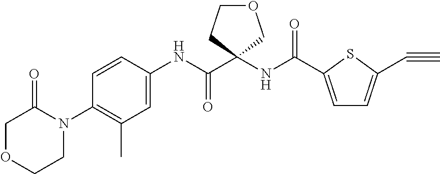

and
(S)-5-bromo-thiophene-2-carboxylic acid-N-{3-[3-methyl-4-(pyrrolidin-1-yl-carbonyl)-phenylcarbamoyl]-tetrahydrofuran-3-yl}-amide

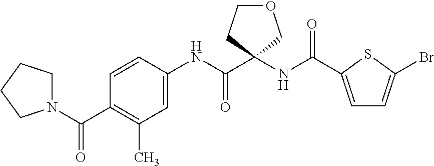

(S)-5-bromo-thiophene-2-carboxylic acid-N-{3-[4-(pyrrolidin-1-yl-carbonyl)-phenylcarbamoyl]-tetrahydrofuran-3-yl}-amide

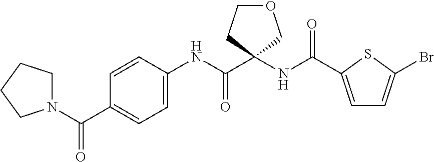

(S)-5-ethinyl-thiophene-2-carboxylic acid-N-{3-[3-chloro-4-(3-oxo-morpholin-4-yl)-phenylcarbamoyl]-tetrahydro-thiophen-3-yl}-amide,

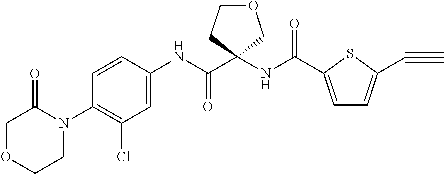

(S)-5-bromo-thiophene-2-carboxylic acid-N-{3-[3-chloro-4-(2-oxo-azepan-1-yl)-phenylcarbamoyl]-tetrahydro-thiophen-3-yl}-amide,

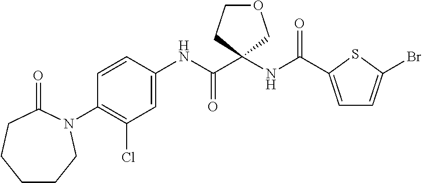

(S)-5-bromo-thiophene-2-carboxylic acid-N-{3-[4-(2-oxo-azepan-1-yl)-phenylcarbamoyl]-tetrahydro-thiophen-3-yl}-amide,

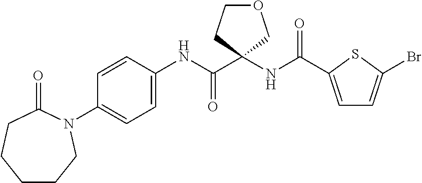

(S)-5-bromo-thiophene-2-carboxylic acid-N-{3-[3-methyl-4-(5-cyanimin-[1.4]oxazepan-4-yl)-phenylcarbamoyl]-tetrahydrofuran-3-yl}-amide

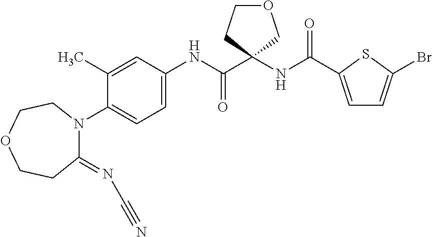

(S)-5-bromo-thiophene-2-carboxylic acid-N-{3-[3-chloro-4-(5-cyanimin-[1.4]oxazepan-4-yl)-phenylcarbamoyl]-tetrahydrofuran-3-yl}-amide

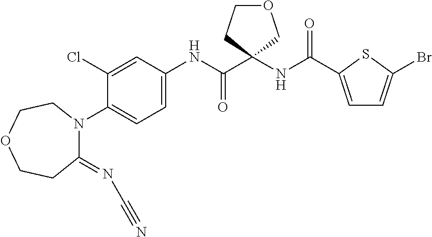

(S)-5-chloro-thiophene-2-carboxylic acid-N-{3-[3-methyl-4-(3-cyanimino-morpholin-4-yl)-phenylcarbamoyl]-tetrahydrofuran-3-yl}-amide

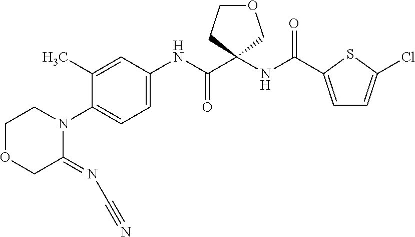

(S)-5-chloro-thiophene-2-carboxylic acid-N-{3-[3-chloro-4-(3-cyanimino-morpholin-4-yl)-phenylcarbamoyl]-tetrahydrofuran-3-yl}-amide

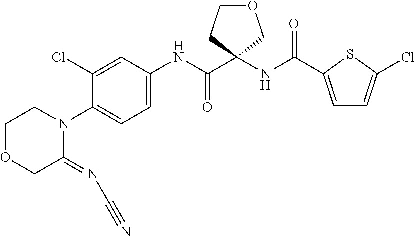

(S)-3-[(5-chloro-thiophen-2-yl)-carbonylamino]-N-((2S)-2,3-dimethyl-2,3,4,5-tetrahydro-1H-benzo[d]azepin-7-yl)-tetrahydrofuran-3-carboxylic acid amide and
(S)-3-[(5-chloro-thiophen-2-yl)-carbonylamino]-N-((2R)-2,3-dimethyl-2,3,4,5-tetrahydro-1H-benzo[d]azepin-7-yl)-tetrahydrofuran-3-carboxylic acid amide

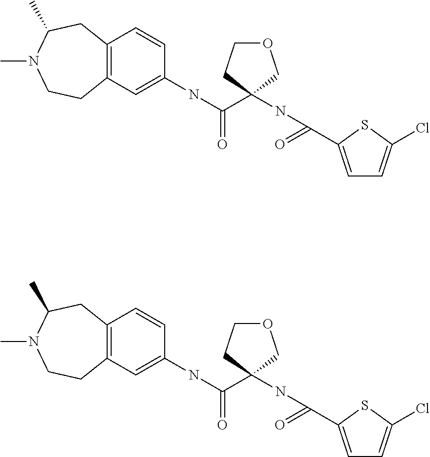

(S)-3-[(5-chloro-thiophen-2-yl)-carbonylamino]-N-((4S)-3,4-dimethyl-2,3,4,5-tetrahydro-1H-benzo[d]azepin-7-yl)-tetrahydrofuran-3-carboxylic acid amide and
(S)-3-[(5-chloro-thiophen-2-yl)-carbonylamino]-N-((2R)-3,4-dimethyl-2,3,4,5-tetrahydro-1H-benzo[d]azepin-7-yl)-tetrahydrofuran-3-carboxylic acid amide

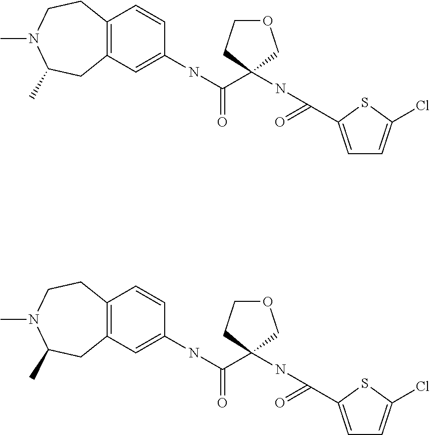

the following compound,

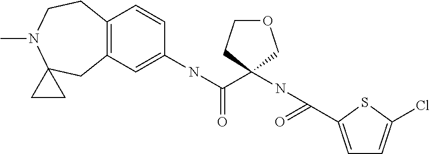

the following compound,

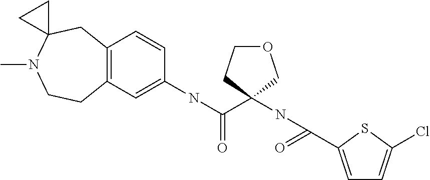

(S)-3-[(5-chloro-thiophen-2-yl)-carbonylamino]-N-(2,2,3-trimethyl-2,3,4,5-tetrahydro-1H-benzo[d]azepin-7-yl)-tetrahydrofuran-3-carboxylic acid amide

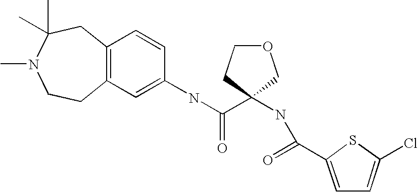

(S)-3-[(5-chloro-thiophen-2-yl)-carbonylamino]-N-(3,4,4-trimethyl-2,3,4,5-tetrahydro-1H-benzo[d]azepin-7-yl)-tetrahydrofuran-3-carboxylic acid amide

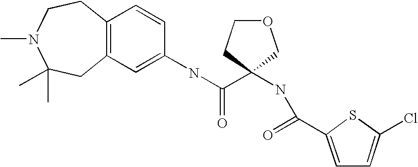

(S)-3-[(5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide

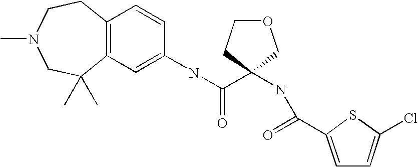

(S)-3-[(5-chloro-thiophen-2-yl)-carbonylamino]-N-(3-methyl-9-fluor-2,3,4,5-tetrahydro-1H-benzo[d]azepin-7-yl)-tetrahydrofuran-3-carboxylic acid amide

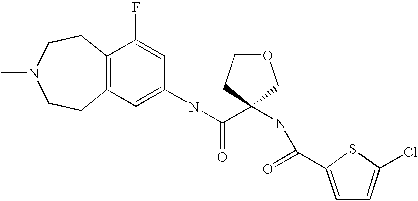

(S)-3-[(5-chloro-thiophen-2-yl)-carbonylamino]-N-(3-methyl-8-fluor-2,3,4,5-tetrahydro-1H-benzo[d]azepin-7-yl)-tetrahydrofuran-3-carboxylic acid amide

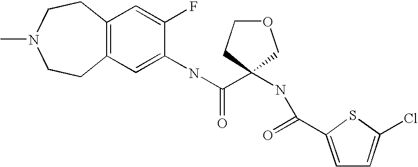

as well as
(R)-3-[(5-bromo-thiophen-2-yl)-carbonylamino]-N-(3-methyl-2,3,4,5-tetrahydro-1H-benzo[d]azepin-7-yl)-tetrahydrofuran-3-carboxylic acid amide,
(R)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide,
(3R)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and
(3R)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide,
(R)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide,
(R)-5-chloro-thiophene-2-carboxylic acid-N-{3-[4-(5-cyanimin-[1.4]oxazepan-4-yl)phenylcarbamoyl]-tetrahydrofuran-3-yl}-amide,
(R)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide,
(R)-5-bromo-thiophene-2-carboxylic acid-N-{3-[4-(5-cyanimin-[1.4]oxazepan-4-yl)phenylcarbamoyl]-tetrahydrofuran-3-yl}-amide,
(R)-5-bromo-thiophene-2-carboxylic acid-N-{3-[3-methyl-4-(3-oxo-morpholin-4-yl)-phenylcarbamoyl]-tetrahydro-thiophen-3-yl}-amide,
(R)-5-ethinyl-thiophene-2-carboxylic acid-N-{3-[4-(3-oxo-morpholin-4-yl)-phenylcarbamoyl]-tetrahydro-thiophen-3-yl}-amide,
(R)-5-bromo-thiophene-2-carboxylic acid-N-{3-[3-methyl-4-(pyrrolidin-1-yl-carbonyl)-phenylcarbamoyl]-tetrahydrofuran-3-yl}-amide,
(R)-5-bromo-thiophene-2-carboxylic acid-N-{3-[4-(pyrrolidin-1-yl-carbonyl)-phenylcarbamoyl]-tetrahydrofuran-3-yl}-amide,
(R)-5-ethinyl-thiophene-2-carboxylic acid-N-{3-[3-chloro-4-(3-oxo-morpholin-4-yl)-phenylcarbamoyl]-tetrahydro-thiophen-3-yl}-amide,
(R)-5-bromo-thiophene-2-carboxylic acid-N-{3-[3-chloro-4-(2-oxo-azepan-1-yl)-phenylcarbamoyl]-tetrahydro-thiophen-3-yl}-amide,
(R)-5-bromo-thiophene-2-carboxylic acid-N-{3-[4-(2-oxo-azepan-1-yl)-phenylcarbamoyl]-tetrahydro-thiophen-3-yl}-amide,
(R)-5-bromo-thiophene-2-carboxylic acid-N-{3-[3-methyl-4-(5-cyanimin-[1.4]oxazepan-4-yl)-phenylcarbamoyl]-tetrahydrofuran-3-yl}-amide,
(R)-5-bromo-thiophene-2-carboxylic acid-N-{3-[3-chloro-4-(5-cyanimin-[1.4]oxazepan-4-yl)-phenylcarbamoyl]-tetrahydrofuran-3-yl}-amide,
(R)-5-chloro-thiophene-2-carboxylic acid-N-{3-[3-methyl-4-(3-cyanimino-morpholin-4-yl)-phenylcarbamoyl]-tetrahydrofuran-3-yl}-amide,
(R)-5-chloro-thiophene-2-carboxylic acid-N-{3-[3-chloro-4-(3-cyanimino-morpholin-4-yl)-phenylcarbamoyl]-tetrahydrofuran-3-yl}-amide,
   the following compound

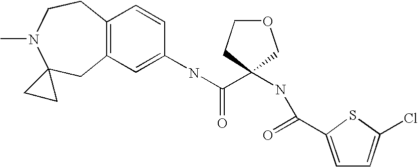

the following compound

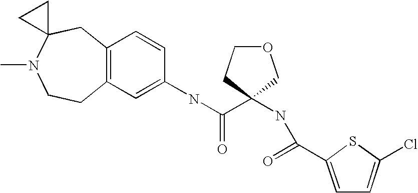

(R)-3-[(5-chloro-thiophen-2-yl)-carbonylamino]-N-(2,2,3-trimethyl-2,3,4,5-tetrahydro-1H-benzo[d]azepin-7-yl)-tetrahydrofuran-3-carboxylic acid amide,
(R)-3-[(5-chloro-thiophen-2-yl)-carbonylamino]-N-(3,4,4-trimethyl-2,3,4,5-tetrahydro-1H-benzo[d]azepin-7-yl)-tetrahydrofuran-3-carboxylic acid amide,
(R)-3-[(5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide,
(R)-3-[(5-chloro-thiophen-2-yl)-carbonylamino]-N-(3-methyl-9-fluor-2,3,4,5-tetrahydro-1H-benzo[d]azepin-7-yl)-tetrahydrofuran-3-carboxylic acid amide, and
(R)-3-[(5-chloro-thiophen-2-yl)-carbonylamino]-N-(3-methyl-8-fluor-2,3,4,5-tetrahydro-1H-benzo[d]azepin-7-yl)-tetrahydrofuran-3-carboxylic acid amide.

In a preferred embodiment, the following compounds are preferred:
(S)-3-[(5-bromo-thiophen-2-yl)-carbonylamino]-N-(3-methyl-2,3,4,5-tetrahydro-1H-benzo[d]azepin-7-yl)-tetrahydrofuran-3-carboxylic acid amide

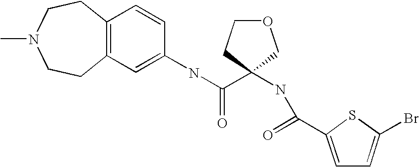

(S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

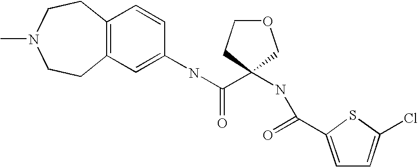

(3S)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide

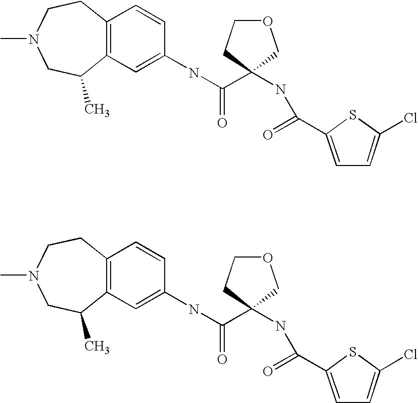

(3S)-3-[(5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amide

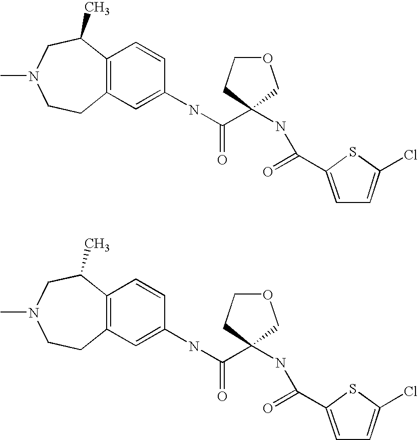

(S)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide

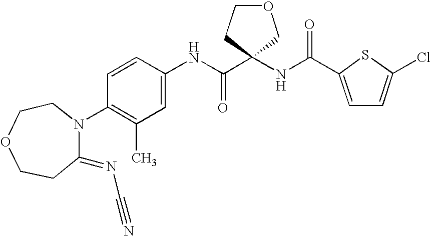

(S)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide

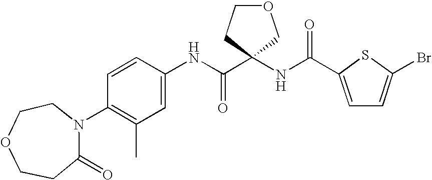

(S)-5-chloro-thiophene-2-carboxylic acid-N-{3-[4-(5-cyanimin-[1.4]oxazepan-4-yl)-phenylcarbamoyl]-tetrahydrofuran-3-yl}-amide

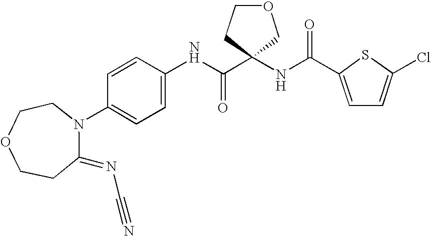

(S)-5-bromo-thiophene-2-carboxylic acid-N-{3-[3-methyl-4-(3-oxo-morpholin-4-yl)-phenylcarbamoyl]-tetrahydro-thiophen-3-yl}-amide

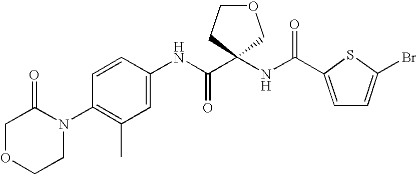

(S)-3-[(5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide

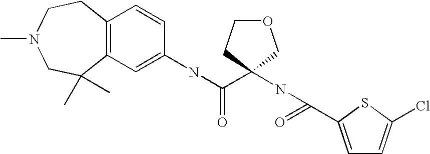

The invention also relates to physiologically acceptable salts of the compounds according to the embodiments defined above and the Examples.

### Description of the Manufacturing Process of the Invention

### a) The Preparation of a Compound of General Formula (Ia)

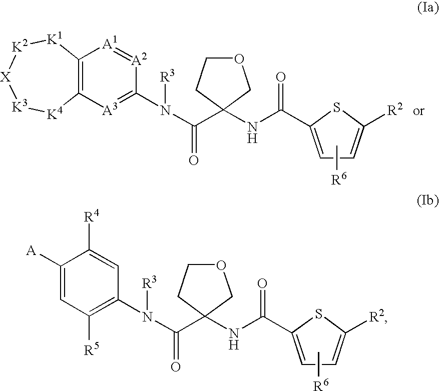

wherein A, A¹ to A³, K¹ to K⁴, X and R¹ to R⁶ are defined as in embodiment 1, and which may optionally be protected at any amino, hydroxy, or carboxy groups present by the usual protective groups such as for example those described in T. W. Greene, P. G. M. Wuts in “Protective Groups in Organic Synthesis” and the protective groups of which may be cleaved in a manner known from the literature,
is described in the exemplifying embodiments or can be done according to known procedures from the literature or may be carried out for example according to one of the following formula schemes 1a or 1b or 2:

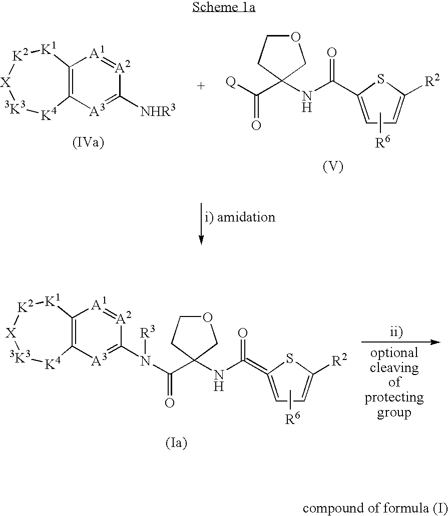

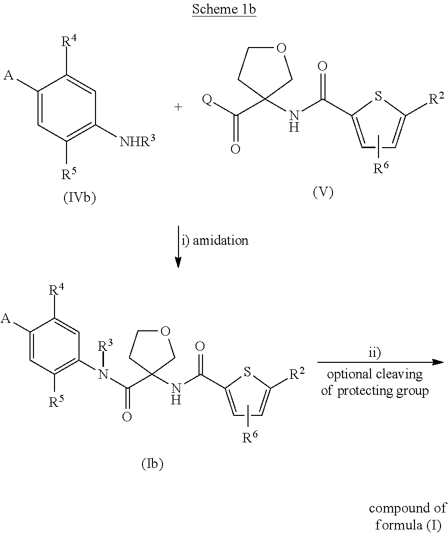

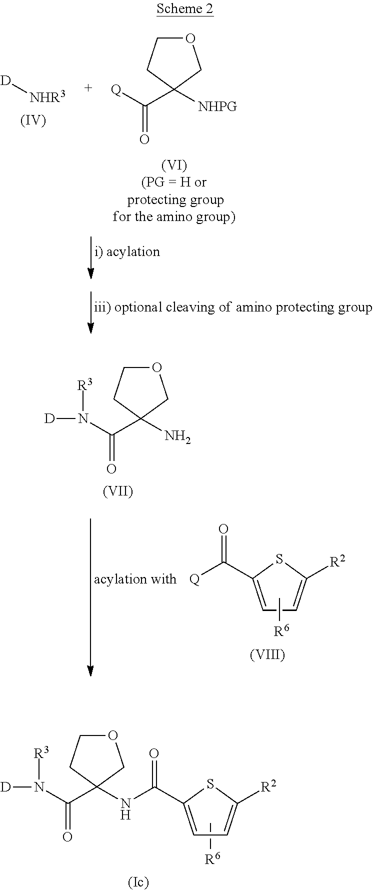

where
Q denotes a hydroxy or C₁₋₄-alkyloxy group, a halogen atom or a C₁₋₅-alkyloxycarbonyloxy or acyloxy group and
PG denotes a hydrogen atom or a protective group for the amino function known from the literature such as for example a tert.-butoxycarbonyl, benzyloxycarbonyl, p-methoxybenzyloxycarbonyl, allyloxycarbonyl, ethyloxycarbonyl, isopropyloxycarbonyl, 2,2,2-trichlorethyloxycarbonyl, methyloxycarbonyl, 9-fluorenylmethyloxycarbonyl, 2-trimethylsilylethyloxycarbonyl, phenylethyloxycarbonyl, acetyl or a trifluoroacetyl group.

Enantiomerically pure compounds Ia, Ib and Ic can be obtained either by chiral chromatography or chemical resolution of racemic Ia, Ib or Ic or optically active intermediates V, VI or VII can be employed in the synthetic steps described in Scheme 1a, 1b and 2.

Accordingly, in a further embodiment, the present invention includes a method for the preparation of substituted 3-amino-tetrahydrofuran-3-carboxylic acid amides of general formula (Ia) in high optical purity at the carbon in position 3 of the tetrahydrofuran ring comprising reacting a compound of the general formula (IVa) with a compound of the general formula (V) in high optical purity at the carbon in position 3 of the tetrahydrofuran ring optionally further comprising cleaving protecting groups, wherein K1, K2, K3, K4, X, A1, A2, A3, R2, R3 and R6, and Q are as defined hereinabove. The amino-tetrahydrofuran carboxylic acid amide moiety of the compound of the general formula (V) and of the 3-amino-tetrahydrofuran-3-carboxylic acid amides of general formula (Ia) may have the R-configuration or the S-configuration.

In a further embodiment, the present invention also includes a method for the preparation of substituted 3-amino-tetrahydrofuran-3-carboxylic acid amides of general formula (Ib) in high optical purity at the carbon in position 3 of the tetrahydrofuran ring comprising reacting a compound of the general formula (IVb) with a compound of the general formula (V) in high optical purity at the carbon in position 3 of the tetrahydrofuran ring, optionally further comprising cleaving protecting groups, wherein A, R4, R5, R2, R3 and R6, and Q are as defined hereinabove. The amino-tetrahydrofuran carboxylic acid amide moiety of the compound of the general formula (V) and of the 3-amino-tetrahydrofuran-3-carboxylic acid amides of general formula (Ib) may have the R-configuration or the S-configuration.

In a further embodiment, the present invention also includes a method for the preparation of substituted 3-amino-tetrahydrofuran-3-carboxylic acid amides of general formula (Ic) in high optical purity at the carbon in position 3 of the tetrahydrofuran ring comprising the steps of: a) reacting a compound of the formula (IV) with a compound of the formula (VI) in high optical purity at the carbon in position 3 of the tetrahydrofuran ring to obtain a compound of the formula (VII) in high optical purity at the carbon in position 3 of the tetrahydrofuran ring, optionally cleaving the amino protecting group; and b) reacting said compound (VII) of step a) with a compound of formula (VIII), wherein Q, PG, D, R3, R2 and R6 are as defined hereinabove. The amino-tetrahydrofuran carboxylic acid amide moiety of the compound of the general formula (VI) and of the 3-amino-tetrahydrofuran-3-carboxylic acid amides of general formula (Ic) may have the R-configuration or the S-configuration.

The reaction steps i)-iii) described in Scheme 1 and 2 may for example be carried out as described in the Examples or under conditions known from the literature, for example as follows:

i) acylation of an amine (IV) or (VII) with an optionally activated carboxylic acid (V) or (VI) or (VIII)

The acylation is expediently carried out with a corresponding halide or anhydride in a solvent such as methylene chloride, chloroform, carbon tetrachloride, ether, ethyl acetate, tetrahydrofuran, dioxane, benzene, toluene, acetonitrile, dimethylformamide, sodium hydroxide solution or sulpholane, optionally in the presence of an inorganic or organic base at temperatures between −20 and 200° C., but preferably at temperatures between −10 and 160° C.

The acylation may however also be carried out with the free acid, optionally in the presence of an acid-activating agent or a dehydrating agent, for example in the presence of isobutyl chloroformate, thionyl chloride, trimethylchlorosilane, hydrogen chloride, sulphuric acid, methanesulphonic acid, p-toluenesulphonic acid, phosphorus trichloride, 1-propylphosphonic acid cyclic anhydride, phosphorus pentoxide, 2-ethoxy-1-ethoxycarbonyl-1,2-dihydroquinoline (EEDQ), N,N′-dicyclohexylcarbodiimide, N,N′-dicyclohexylcarbodiimide/camphorsulphonic acid, N,N′-dicyclohexylcarbodiimide/N-hydroxysuccinimide or 1-hydroxy-benzotriazole, N,N′-carbonyldiimidazole, O-(benzotriazol-1-yl)-N,N,N′,N′-tetramethyl-uronium tetrafluoroborate/N-methylmorpholine, O-(benzotriazol-1-yl)-N,N,N′,N′-tetramethyluronium tetrafluoroborate/N-ethyldiisopropylamine, O-pentafluorophenyl-N,N,N′,N′-tetramethyluronium-hexafluorophosphate/triethylamine, N,N′-thionyldiimidazole or triphenylphosphine/carbon tetrachloride, at temperatures between −20 and 200° C., but preferably at temperatures between −10 and 160° C.

The acylation may also be carried out with a carboxylic acid ester (V) or (VI) and the amine (IV) by activation with trimethylaluminium.

The acylation of a compound of general formula (IV) may however also be carried out with a reactive carboxylic acid derivative of general formula (R- or S-IX)

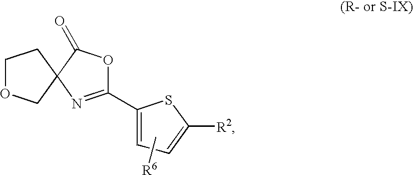

wherein R⁶ and R² are defined as in embodiment 1. The acylation is then conveniently carried out in a solvent such as for example toluene, tetrahydrofuran or dimethylformamide, with the addition of an acid such as acetic acid or camphorsulphonic acid or optionally in the presence of a Lewis acid such as zinc chloride or copper(II)chloride and optionally by the addition of amine bases such as for example diisopropylethylamine, triethylamine or N-methylmorpholine, at temperatures between −10 and 100° C., for example using a microwave oven or as described in P. Wipf et al., Helvetica Chimica Acta, 69, 1986, 1153.

Compounds of general formula (IX) may be prepared from compounds of general formula (V), expediently in a solvent or mixture of solvents such as dichloromethane, trichloromethane, carbon tetrachloride, benzene, chlorobenzene, toluene, xylene, hexamethyldisiloxane, ether, tetrahydrofuran, dioxane, acetonitrile, pyridine, optionally in the presence of N,N′-dicyclohexylcarbodiimide, N,N′-dicyclohexylcarbodiimide/N-hydroxysuccinimide or 1-hydroxy-benzotriazole, N,N′-carbonyldiimidazole, O-(benzotriazol-1-yl)-N,N,N′,N′-tetramethyl-uronium tetrafluoroborate/N-methylmorpholine,
-(benzotriazol-1-yl)-N,N,N′,N′-tetramethyluronium tetrafluoroborate/N-ethyldiisopropylamine, or in acetic anhydride at temperatures between −20 and 200° C., but preferably at temperatures between −10 and 100° C.

Other methods of amide coupling are described for example in P. D. Bailey, I. D. Collier, K. M. Morgan in “Comprehensive Functional Group Interconversions”, Vol. 5, page 257ff., Pergamon 1995 or in Supplementary Volume 22 to Houben-Weyl, Thieme Verlag, 2003 and literature cited therein.

ii) or iii) Cleaving a Protective Group

The optional subsequent cleaving of any protective group used is carried out hydrolytically, for example, in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether cleavage, e.g. in the presence of iodotrimethylsilane, at temperatures between 0 and 100° C., preferably at temperatures between 10 and 50° C.

A benzyl, methoxybenzyl or benzyloxycarbonyl group may, however, be cleaved hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at room temperature, and under a hydrogen pressure of 1 to 7 bar, but preferably 1 to 5 bar.

A protective group may however also be cleaved by the methods described in T. W. Greene, P. G. M. Wuts in “Protective Groups in Organic Synthesis”.

### (b) The Components of General Formula IV (Including IVa and IVb)

D-NH—R3 (IV),

wherein D and R³ are defined as in embodiment 1,
and which may optionally be protected at any amino, hydroxy, or carboxy groups present by the usual protective groups such as for example those described in T. W. Greene, P. G. M. Wuts in “Protective Groups in Organic Synthesis” and the protective groups of which may be cleaved in a manner known from the literature in the course of the synthesis sequence to form compounds of formula (I),
are known from the literature, or their synthesis is described in the exemplifying embodiments, or they may be prepared for example using methods of synthesis known from the literature or analogously to methods of synthesis known from the literature as described for example in DE4429079, U.S. Pat. No. 4,490,369, DE3515864, U.S. Pat. No. 5,175,157, DE1921861, WO85/00808 or in G. Bobowski et al., J. Heterocyclic Chem. 16, 1525, 1979 or in P. D. Johnson et al., Bioorg. Med. Chem. Letter 2003, 4197, or in WO04/46138, WO05/111014, WO05/111029 or WO06/34822.

Fragments bridged in the azepine moiety as shown in formula II-1 or II-2 may for example be prepared analogously to J. W. Coe et al. J. Med. Chem., 2005, 48, 3474 or J. W. Coe et al., US Patent application US2005/0020616, WO05/111014, WO05/111029 or WO06/34822.

For example, a compound of general formula (IV), wherein R³ denotes a hydrogen atom and A1, A2, A3, K1, K2, K3, K4 and X are defined as in embodiment 1, may be prepared by reduction of the nitro group of a compound of general formula (III)

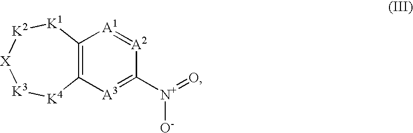

wherein A1, A2, A3, K1, K2, K3, K4 and X are defined as in embodiment 1, as follows.

The reduction of the nitro group is for example conveniently carried out in a solvent or mixture of solvents such as water, aqueous ammonium chloride solution, hydrochloric acid, sulphuric acid, phosphoric acid, formic acid, acetic acid, acetic anhydride with base metals such as iron, zinc, tin or sulphur compounds such as ammonium sulphide, sodium sulphide or sodium dithionite or by catalytic hydrogenation with hydrogen, for example under a pressure between 0.5 and 100 bar, but preferably between 1 and 50 bar, or with hydrazine as reducing agent, conveniently in the presence of a catalyst such as for example Raney nickel, palladium charcoal, platinum oxide, platinum on mineral fibres or rhodium, or with complex hydrides such as lithium aluminium hydride, sodium borohydride, sodium cyanoborohydride, diisobutylaluminium hydride, conveniently in a solvent or mixture of solvents such as water, methanol, ethanol, isopropanol, pentane, hexane, cyclohexane, heptane, benzene, toluene, xylene, ethyl acetate, methylpropionate, glycol, glycoldimethylether, diethyleneglycoldimethylether, dioxane, tetrahydrofuran, N-methylpyrrolidinone, or N-ethyl-diisopropylamine, N—C₁₋₅-alkylmorpholine, N—C₁₋₅-alkylpiperidine, N—C₁₋₅-alkylpyrrolidine, triethylamine, pyridine, for example at temperatures between −30 and 250° C., but preferably between 0 and 150° C.

### (c) The Components of General Formula

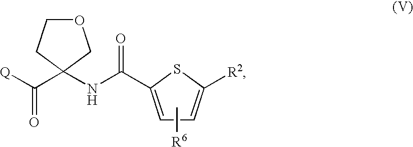

wherein R⁴, R⁵, R⁶ and R² are defined as in embodiment 1, and where Q denotes a hydroxy or C₁₋₄-alkyloxy group, a halogen atom or a C₁₋₅-alkyloxycarbonyloxy or acyloxy group
which may optionally be protected at any amino, hydroxy, carboxy or thiol groups present by the usual protective groups such as for example those described in T. W. Greene, P. G. M. Wuts in “Protective Groups in Organic Synthesis” and the protective groups of which may be cleaved in a manner known from the literature in the course of the synthesis sequence to form compounds of formula (I),
are known from the literature, or their synthesis is described in the exemplifying embodiments, or they may be prepared for example using methods of synthesis known from the literature or analogously to methods of synthesis known from the literature as described for example in WO04/46138, WO05/111014, WO05/111029 or WO06/34822.

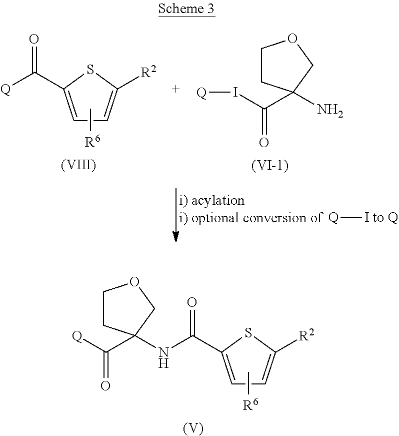

For example they may also be prepared according to Scheme 3 by reacting a compound (VIII) with an amine (VI-1), where Q denotes a hydroxy or C₁₋₄-alkyloxy group, a halogen atom or an alkyloxycarbonyloxy or acyloxy group and Q-I denotes a hydroxy or C₁₋₄-alkyloxy group, which may optionally be converted into Q after the acylation step by saponification and activation as described above. The acylation may be carried out according to the acylation conditions described above.

The amino acid derivatives (VI-1) are known from the literature or may be prepared analogously to methods known from the literature as described in the Examples, for example, from commercially obtainable amino acid derivatives.

### d) The Components of General Formula V or

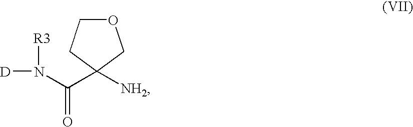

wherein D and R³ are defined as in embodiment 1,
can be synthesized as described in the exemplifying embodiments, or they may be prepared for example using methods of synthesis known from the literature or they may be prepared according to Scheme 4.

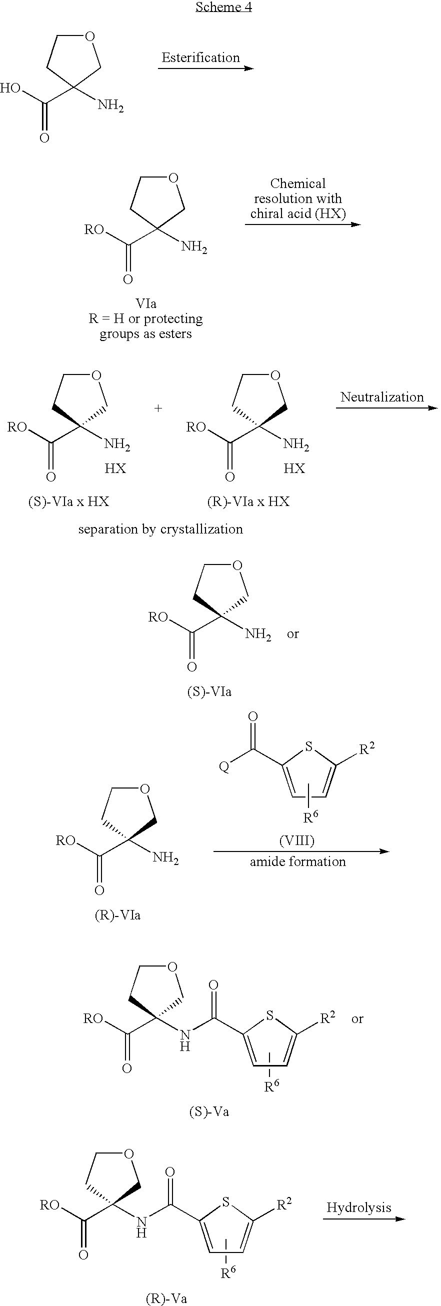

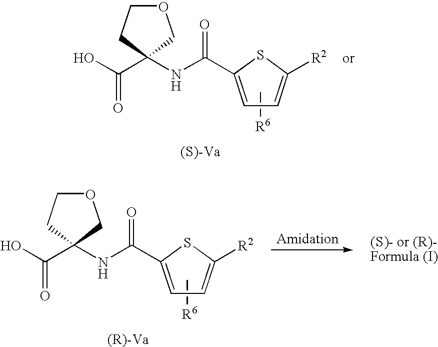

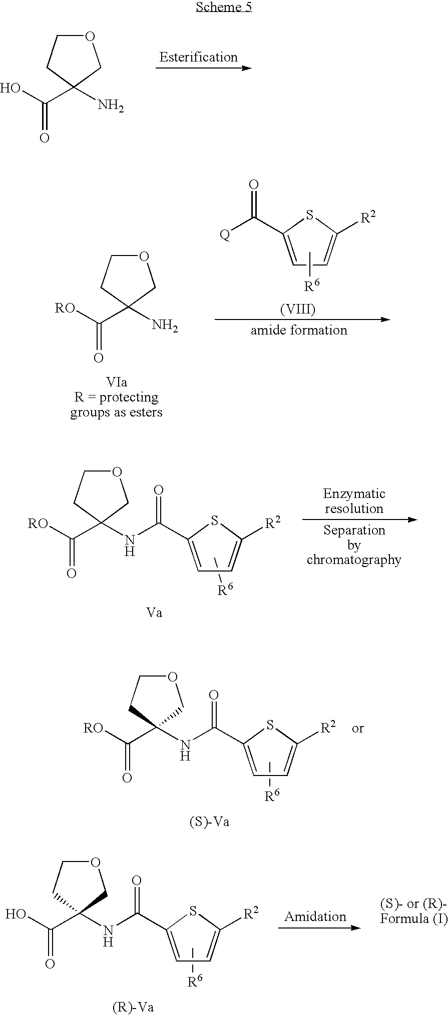

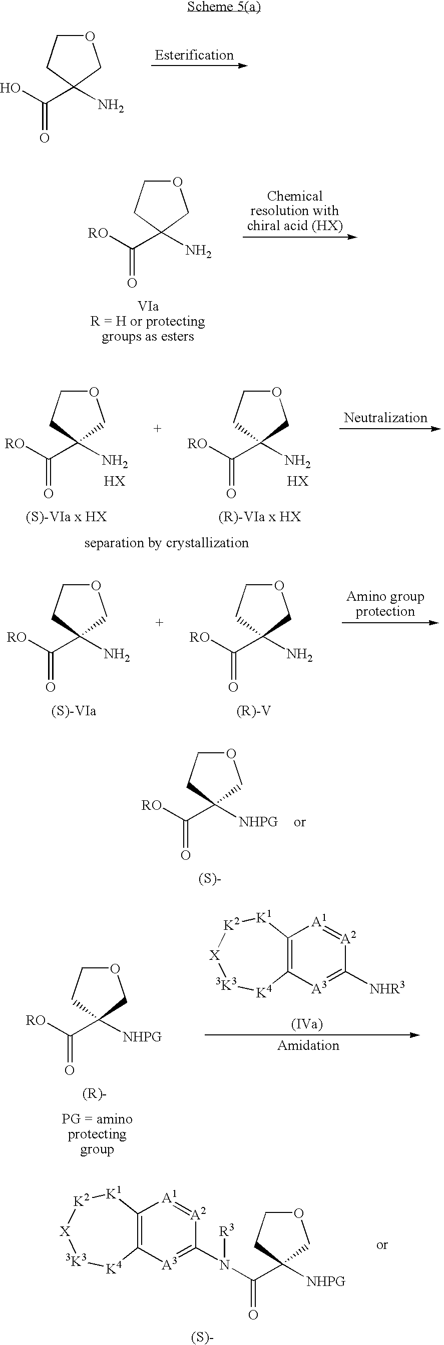

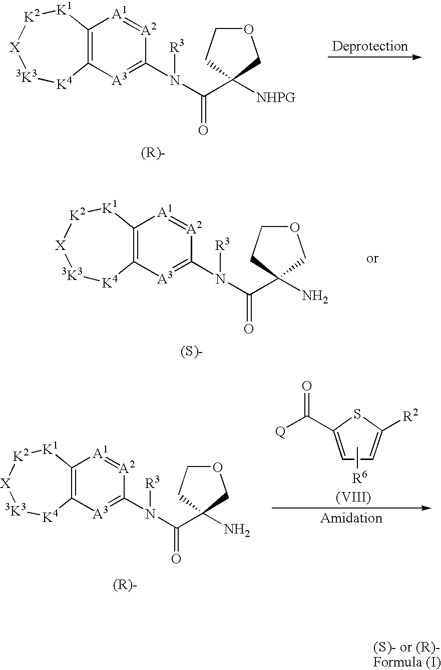

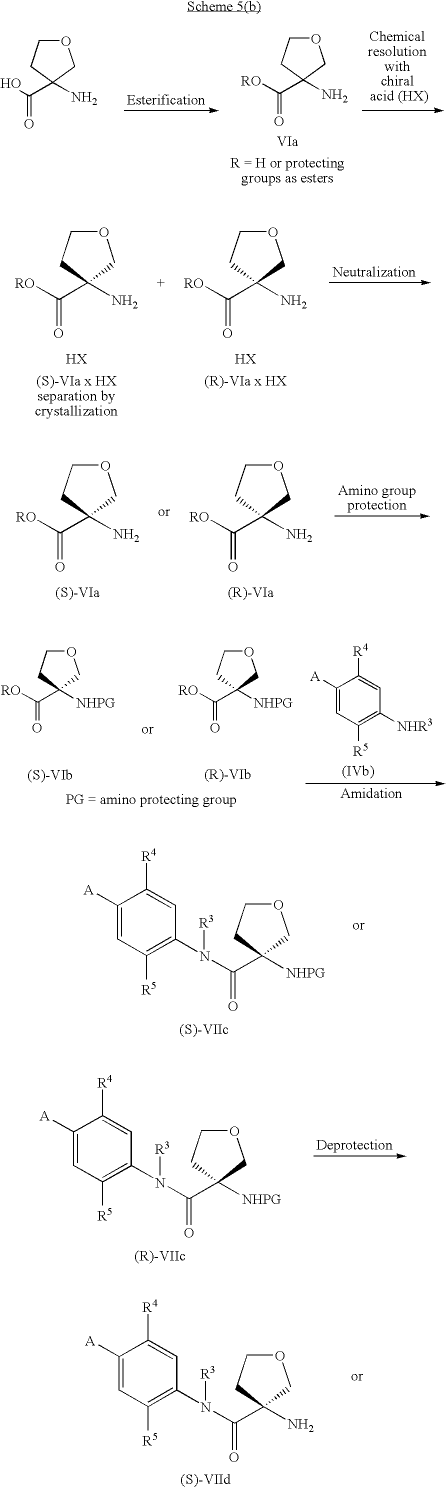

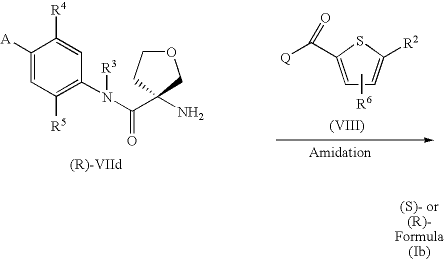

### e) Preparation of a Compound of General Formula (XI)

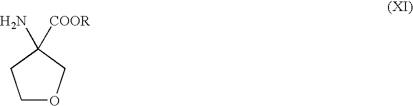

wherein R is C₁-C₁₂-alkyl, aryl, or aryl-C₁-C₁₂-alkyl or a heterocycle, for example R is C₁-C₄-alkyl, aryl, or aryl-C₁-C₄-alkyl, alternatively R is methyl, n-butyl or iso-butyl, benzyl, phenethyl.

In one embodiment, the present invention relates to a process for the preparation of a compound of the formula (XI). In this process, a Strecker reaction is used in a “one-pot” process followed by in suit formation of ester avoiding the isolation of intermediate product 21 from the reaction mixture. For example, the new process is shown as follow:

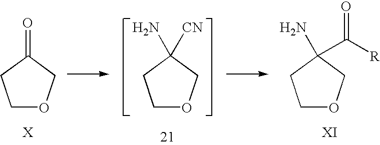

In one embodiment, the process uses ketone (X) as the starting material in a reaction with a nitrogen source (for example ammonium acetate or ammonia), in alcohol (for example methanol or ethanol) and a cyanide source (for example a cyanide salt, such as sodium cyanide or potassium sodium). The reaction is for example carried out at ambient temperature. This results in the production of intermediate 21. Typically, intermediate 21 is not isolated and is directly treated with alcohol R—OH in the presence of acid (for example HCl) to generate the desired ester. The process is generally operated in mild condition and readily available starting materials are used.

Optically pure derivatives of the compounds of the general formula (XI) can be obtained by the resolution methods described hereinbelow, including chemical resolution for example with L-Mandelic acid, or enzymatic resolution, for example with alcalase.

Accordingly, in a further embodiment, the invention relates to a compound of the general formula (XI) in high optical purity. In one aspect the compound of the general formula (XI) in high optical purity is in the R-configuration or in the S-configuration.

In the reactions described hereinbefore any reactive groups present such as hydroxy, carboxy, amino, alkylamino or imino groups may be protected during the reaction by conventional protective groups which are cleaved again after the reaction.

For example a protecting group for a hydroxy group might be the methoxy, benzyloxy, trimethylsilyl, acetyl, benzoyl, tert.-butyl, trityl, benzyl or tetrahydropyranyl group.

Protecting groups for a carboxyl group might be the trimethylsilyl, methyl, ethyl, tert.-butyl, benzyl or tetrahydropyranyl group.

A protecting group for an amino, alkylamino or imino group might be the acetyl, trifluoroacetyl, benzoyl, ethoxycarbonyl, tert.-butoxycarbonyl, benzyloxycarbonyl, benzyl, methoxybenzyl or 2,4-dimethoxybenzyl group and additionally, for the amino group, the phthalyl group.

For example a protecting group for an ethynyl group might be the trimethylsilyl, diphenylmethylsilyl, tert.butyldimethylsilyl or a 1-hydroxy-1-methyl-ethyl group.

Other protective groups which may be used and their removal are described in T. W. Greene, P. G. M. Wuts, “Protective Groups in Organic Synthesis”, Wiley, 1991 and 1999.

Any protective group used is optionally subsequently cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by means of ether splitting, e.g. in the presence of iodotrimethylsilane, at temperatures between 0 and 100° C., preferably at temperatures between 10 and 50° C.

A benzyl, methoxybenzyl or benzyloxycarbonyl group, however, is cleaved by hydrogenolysis, for example, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at room temperature, and under a hydrogen pressure of 1 to 7 bar, but preferably 1 to 5 bar.

A methoxybenzyl group may also be cleaved in the presence of an oxidising agent such as cerium(IV)ammonium nitrate in a solvent such as methylene chloride, acetonitrile or acetonitrile/water at temperatures between 0 and 50° C., but preferably at room temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisol.

A tert.-butyl or tert.-butyloxycarbonyl group is preferably cleaved by treatment with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treatment with a catalytic amount of tetrakis-(triphenylphosphine)-palladium(0), preferably in a solvent such as tetrahydrofuran and preferably in the presence of an excess of a base such as morpholine or 1,3-dimedone at temperatures between 0 and 100° C., preferably at room temperature and under inert gas, or by treatment with a catalytic amount of tris-(triphenylphosphine)-rhodium(I)chloride in a solvent such as aqueous ethanol and optionally in the presence of a base such as 1,4-diazabicyclo[2,2,2]octane at temperatures between 20 and 70° C.

Furthermore, the compounds of formula (I) obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula (I) contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine, and triethanolamine.

According to the invention the optically pure derivatives of 3-Amino-tetrahydrofuran-3-carboxylic acid e.g. I, V, VI, VII or IX can be obtained for example in analogy to the following methods:

Method I: Chemical Resolution with Mandelic Acid

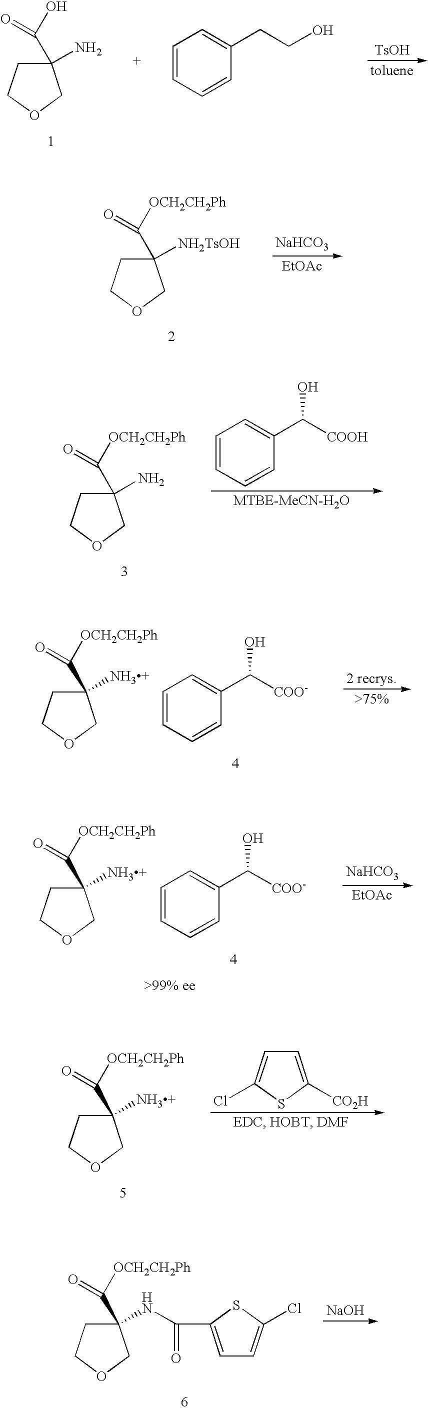

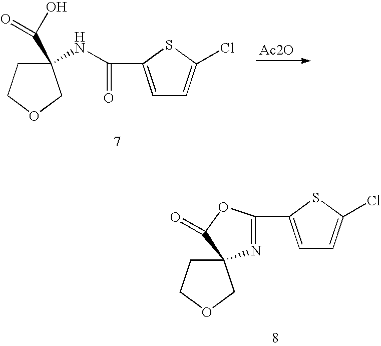

### Method II: Separation of the Enantiomers Via Chiral Chromatography

The separations can be performed on various DAICEL columns like AD-H, OD-H, AS-H, OJ-H, IA, IB and Kromasil DMB, TBB. Especially useful are DAICEL AD-H, OJ-H and IA columns.

Chiral chromatography can be used to

separate racemic 7 into its S- and R-enantiomer

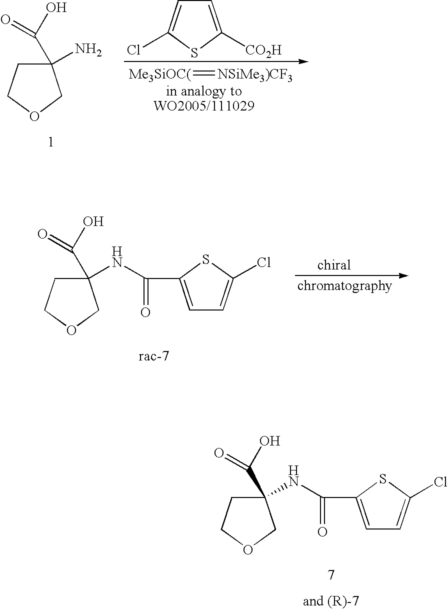

or b) to separate racemic derivatives 11 like

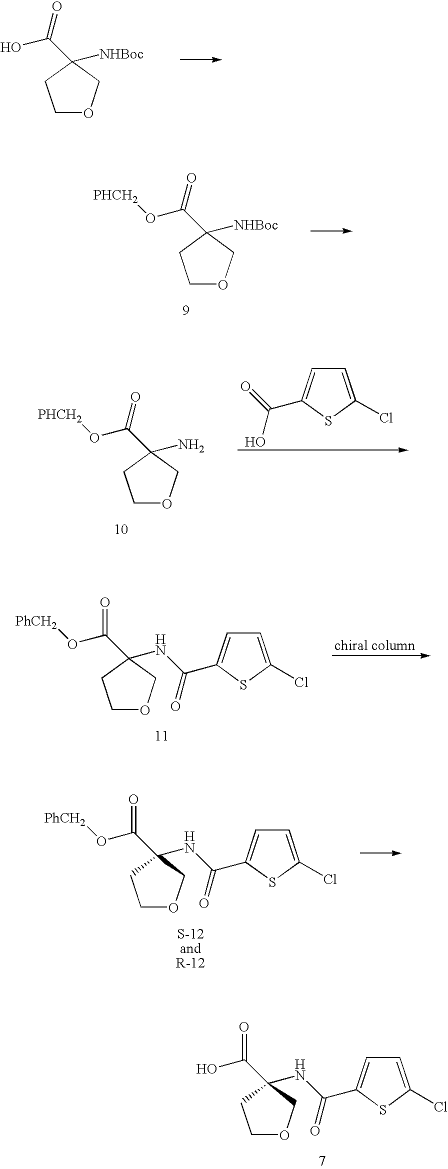

or c) to separate racemic mixtures of I

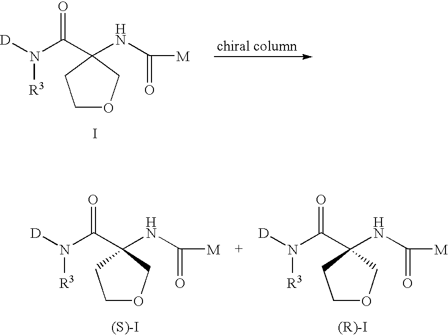

### Method III: Enzymatic Resolution

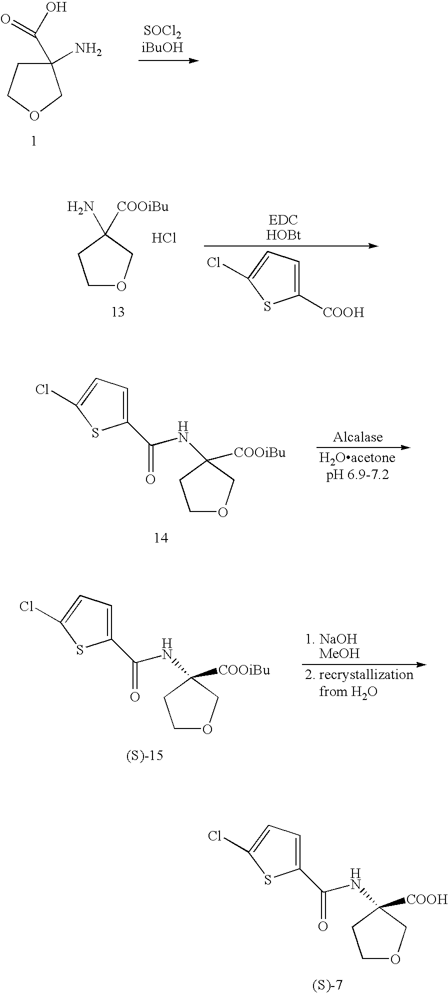

Method IV: Alternative Chemical Resolution with Mandelic Acid

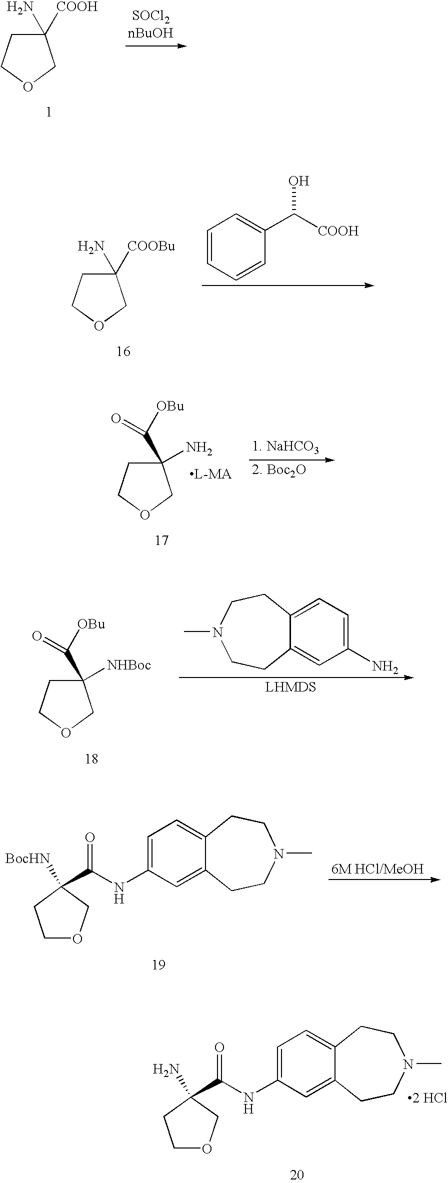

Accordingly, in a further embodiment, the present invention includes a method for preparing a compound of the formula (V) in high optical purity at the carbon in position 3 of the tetrahydrofuran ring by enzymatic resolution of a racemic mixture of said compound of the formula (V), preferably with alcalase, wherein R2 and R6 are as defined hereinabove, and wherein Q is a straight or substituted C₁₋₁₂-alkyloxy group, allyloxy or substituted allyloxy group, a C₁₋₁₂-alkyloxycarbonyloxy or acyloxy group, preferably Q is a straight or substituted C₁₋₄-alkyloxy.

In another embodiment, the present invention includes a compound of the formula (V) in high optical purity at the carbon in position 3 of the tetrahydrofuran ring, wherein R2 and R6 are as defined hereinabove, and wherein Q is a hydroxy or substituted C₁₋₁₂-alkyloxy group, a halogen atom or a C₁₋₁₂-alkyloxycarbonyloxy or acyloxy group, or substituted allyloxy group, preferably Q is a substituted C₁₋₁₂-alkyloxy or substituted allyloxy group. In one aspect of the invention, the amino-tetrahydrofuran carboxylic acid amide moiety of the compound of the general formula (V) in high optical purity at the carbon in position 3 of the tetrahydrofuran ring has the S-configuration. In another aspect, the compound of the general formula (V) in high optical purity at the carbon in position 3 of the tetrahydrofuran ring is obtainable by the process described above.

In a further embodiment, the present invention includes a method for preparing a compound of the formula (VI) in high optical purity at the carbon in position 3 of the tetrahydrofuran ring by enzymatic resolution of a racemic mixture of said compound of the formula (VI), preferably with alcalase, wherein Q is straight or substituted C₁₋₁₂-alkyloxy group, or a C₁₋₁₂-alkyloxycarbonyloxy or acyloxy group, or substituted allyloxy group, preferably Q is a substituted C₁₋₄-alkyloxy group, and PG is a hydrogen atom or a protective group for the amino function as defined hereinabove.

In another embodiment, the present invention includes a compound of the formula (VI) in high optical purity at the carbon in position 3 of the tetrahydrofuran ring, wherein Q is a hydroxy or straight or substituted C₁₋₁₂-alkyloxy group, a halogen atom or a C₁₋₁₂-alkyloxycarbonyloxy or acyloxy group, or substituted allyloxy, preferably Q is a straight or substituted C₁₋₄-alkyloxy group, and PG is a hydrogen atom or a protective group for the amino function as defined hereinabove. In one aspect of the invention, the amino-tetrahydrofuran carboxylic acid amide moiety of the compound of the general formula (VI) in high optical purity at the carbon in position 3 of the tetrahydrofuran ring has the S-configuration. In another aspect, the compound of the general formula (VI) in high optical purity at the carbon in position 3 of the tetrahydrofuran ring is obtainable by the process described above.

In another embodiment, the present invention includes a method for preparing a compound of the formula (VII) in high optical purity at the carbon in position 3 of the tetrahydrofuran ring by chemical resolution of a racemic mixture of said compound of the formula (VII) with a chiral acid, preferably with L-mandelic acid, wherein D and R3 are as defined hereinabove.

In another embodiment, the present invention includes a compound of the formula (VII) in high optical purity at the carbon in position 3 of the tetrahydrofuran ring, wherein D and R3 are as defined hereinabove. In one aspect of the invention, the amino-tetrahydrofuran carboxylic acid amide moiety of the compound of the general formula (VII) in high optical purity at the carbon in position 3 of the tetrahydrofuran ring has the S-configuration. In another aspect, the compound of the general formula (VII) in high optical purity at the carbon in position 3 of the tetrahydrofuran ring is obtainable by the process described above.

Especially useful 3-Amino-tetrahydrofuran-3-carboxylic acid precursors for the manufacturing of substituted 3-amino-tetrahydrofuran-3-carboxylic acid amides of general formula (I) are the following compounds:

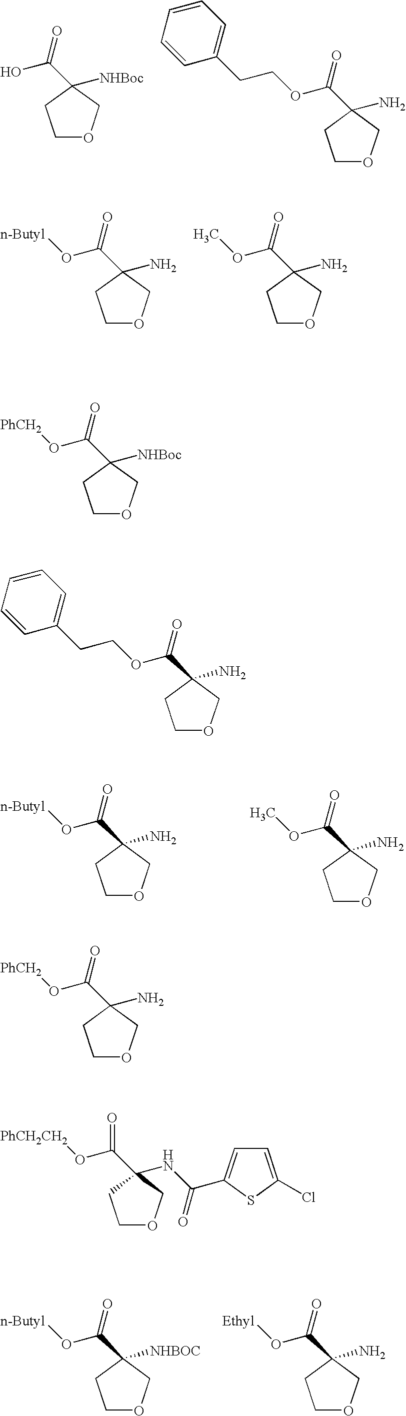

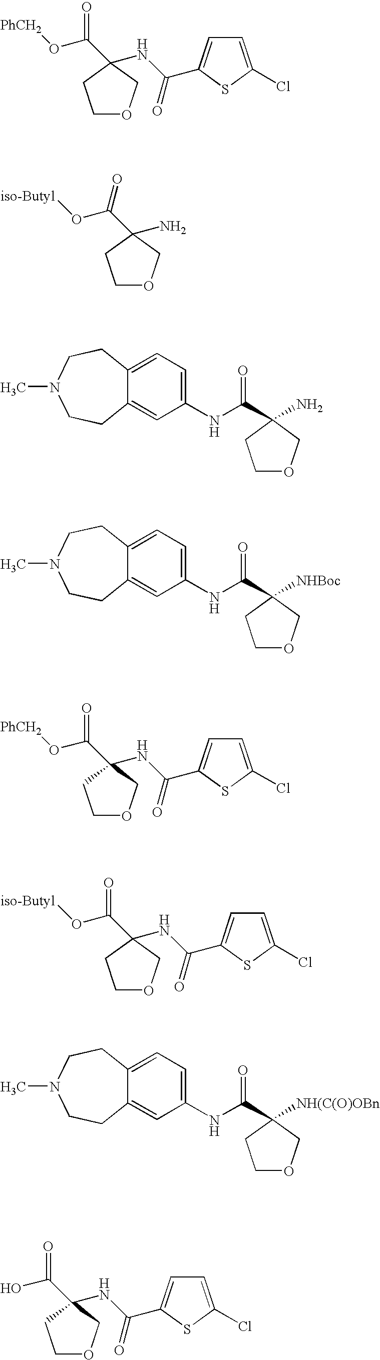

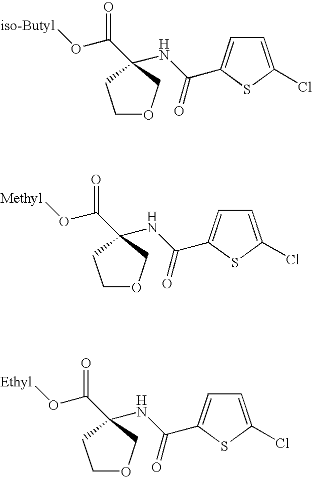

### Determination of the Absolute Stereochemistry

The absolute configuration of the compounds can be determined by X-ray crystallography as is exemplified in the experimental part for (S)-3-[(5-chloro-thiophen-2-yl)-carbonylamino]-tetrahydro-furan-3-carboxylic acid 7:

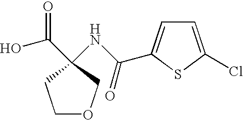

### Description of the Pharmaceutical Useful Properties of the Compounds

As already mentioned hereinbefore, the compounds of general formula (I) and the tautomers, enantiomers, diastereomers and physiologically acceptable salts thereof have valuable pharmacological properties, particularly an antithrombotic activity which is preferably based on an effect on thrombin or factor Xa, for example on a thrombin-inhibiting or factor Xa-inhibiting activity, on a prolonging effect on the aPTT time and on an inhibitory effect on related serine proteases such as e.g. urokinase, factor VIIa, factor IX, factor XI and factor XII.

The compounds listed in the Experimental Section were investigated for their effect on the inhibition of factor Xa as follows:

### Method:

Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

### Material:

Tris(hydroxymethyl)-aminomethane buffer (100 mMol) and sodium chloride (150 mMol), pH 8.0 plus 1 mg/ml Human Albumin Fraction V, protease-free
Factor Xa (Calbiochem), spec. activity: 217 IU/mg, final concentration: 7 IU/ml for each reaction mixture
Substrate S 2765 (Chromogenix), final concentration: 0.3 mM/l (1 KM) for each reaction mixture
Test substance: final concentration 100, 30, 10, 3, 1, 0.3, 0.1, 0.03, 0.01, 0.003, 0.001 μMol/l

### Procedure:

10 μl of a 23.5-times concentrated starting solution of the test substance or solvent (control), 175 μl of TRIS/HSA buffer and 25 μl of a 65.8 U/L Factor Xa working solution are incubated for 10 minutes at 37° C. After the addition of 25 μl of S 2765 working solution (2.82 mMol/l) the sample is measured in a photometer (SpectraMax 250) at 405 nm for 600 seconds at 37° C.

### Evaluation:

1. Determining the maximum increase (deltaOD/minutes) over 21 measuring points.
2. Determining the % inhibition based on the solvent control.
3. Plotting a dosage/activity curve (% inhibition vs substance concentration).
4. Determining the IC₅₀ by interpolating the X-value (substance concentration) of the dosage/activity curve at Y=50% inhibition.

All the compounds tested had an IC₅₀ value of less than 10 μmol/L. Surprisingly, for the compounds with S-configuration at the amino-tetrahydrofuran carboxylic acid amide moiety higher potency was observed in the FXa assay.

For example:

(S)-3-[(5-Bromo-thiophen-2-yl)-carbonylamino]-N-(3-methyl-2,3,4,5-tetrahydro-1H-benzo[d]azepin-7-yl)-tetrahydrofuran-3-carboxylic acid amide showed an IC₅₀ at an approximately 9-fold lower plasma concentration compared to the (R)-enantiomer,
(S)-3-[(5-Chloro-thiophen-2-yl)-carbonylamino]-N-(3-methyl-2,3,4,5-tetrahydro-1H-benzo[d]azepin-7-yl)-tetrahydrofuran-3-carboxylic acid amide showed an IC₅₀ at an approximately 8-fold lower plasma concentration compared to the (R)-enantiomer,
(3S)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide showed an IC₅₀ at an approximately 10-fold lower plasma concentration compared to the corresponding (3R,5R)-diastereoisomer and
(3S)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide showed an IC₅₀ at an approximately 7-fold lower plasma concentration compared to the corresponding (3R,5S)-diastereoisomer.
(S)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide showed an IC₅₀ at an approximately 17-fold lower plasma concentration compared to the (R)-enantiomer,
(S)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide showed an IC₅₀ at an approximately 16-fold lower plasma concentration compared to the (R)-enantiomer,
(S)-5-chloro-thiophene-2-carboxylic acid-N-{3-[4-(5-cyanimin-[1.4]oxazepan-4-yl)-phenylcarbamoyl]-tetrahydrofuran-3-yl}-amide showed an IC₅₀ at an approximately 15-fold lower plasma concentration compared to the (R)-enantiomer and
(S)-5-bromo-thiophene-2-carboxylic acid-N-{3-[3-methyl-4-(3-oxo-morpholin-4-yl)phenylcarbamoyl]-tetrahydro-thiophen-3-yl}-amide showed an IC₅₀ at an approximately 15-fold lower plasma concentration compared to the (R)-enantiomer.

In view of their pharmacological properties the new compounds and the physiologically acceptable salts thereof are suitable for the prevention and treatment of venous and arterial thrombotic diseases, such as for example the prevention and treatment of deep leg vein thrombosis, thrombophlebitis, for preventing reocclusions after bypass operations or angioplasty (PT(C)A), and occlusion in peripheral arterial diseases, and for preventing and treating pulmonary embolism, disseminated intravascular coagulation and severe sepsis, for preventing and treating DVT in patients with exacerbated COPD, for treating ulcerative colitis, for preventing and treating coronary thrombosis, for preventing thromboembolic events associated with atrial fibrillation, e.g., stroke and the occlusion of shunts.

In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic events in patients with all forms of coronary heart disease, for preventing metastasis and the growth of tumours and inflammatory processes, e.g. in the treatment of pulmonary fibrosis or pulmonary arterial hypertension, for preventing and treating rheumatoid arthritis, for preventing and treating fibrin-dependent tissue adhesions and/or the formation of scar tissue and for promoting wound healing processes.

Compounds may also have utility as anticoagulant agents in connection with the preparation, storage fractionation or use of whole blood; and in the coating of invasive devices such as prostheses, artificial valves and catheters in reducing the risk of thrombus formation.

In view of their pharmacological properties the new compounds and the physiologically acceptable salts thereof are also suitable for the treatment of Alzheimer's and Parkinson's disease. One explanation for this arises for example from the following findings, from which it can be concluded that thrombin inhibitors or factor Xa inhibitors, by inhibiting thrombin formation or thrombin activity, may be valuable drugs for treating Alzheimer's and Parkinson's disease. Clinical and experimental studies indicate that neurotoxic mechanisms, for example the inflammation which is associated with the activation of proteases of the clotting cascade, are involved in the dying of neurones following brain injury. Various studies point to the involvement of thrombin in neurodegenerative processes, for example following a stroke, repeated bypass operations or traumatic brain injury. An increased thrombin activity has been demonstrated some days after peripheral nerve damage, for example. It has also been shown that thrombin causes a neurite retraction, as well as glia proliferation, and apoptosis in primary cultures of neurones and neuroblastoma cells (for a summary see: Neurobiol. Aging 2004, 25(6), 783-793). Moreover, various in vitro studies on the brains of patients with Alzheimer's disease indicated that thrombin plays a role in the pathogenesis of this disease (Neurosci. Lett. 1992, 146, 152-54). A concentration of immune-reactive thrombin has been detected in neurite plaques in the brains of Alzheimer's patients. It has been demonstrated in vitro that thrombin also plays a part in the regulation and stimulation of the production of the “Amyloid Precursor Protein” (APP) as well as in the cleaving of the APP into fragments which can be detected in the brains of Alzheimer's patients. Moreover, it has been demonstrated that the thrombin-induced microglial activation leads in vivo to the degeneration of nigral dopaminergic neurones. These findings lead one to conclude that microglial activation, triggered by endogenous substance(s) such as thrombin, for example, are involved in the neuropathological process of the cell death of dopaminergic neurones of the kind which occurs in patients with Parkinson's disease (J. Neurosci. 2003, 23, 5877-86).

The new compounds and the physiologically acceptable salts thereof are also suitable for the prophylaxis and treatment of arterial vascular diseases as combination therapy with lipid lowering agents such as HMG-CoA reductase inhibitors; and vasodilators, in particular ACE-inhibitors, angiotensin II antagonists, renin inhibitors, β-receptor antagonists, α-receptor antagonists, diuretics, Ca-channel blockers, or stimulators of soluble guanylate cyclase.

By increasing the antithrombotic efficacy, the new compounds and the physiologically acceptable salts thereof are also suitable in combination therapy with other anticoagulants e.g. unfractionated heparin, low molecular heparins or fondaparinux, or direct thrombin inhibitors e.g. recombinant hirudin or small molecule synthetic inhibitors.

Similarly, the compounds and the physiologically acceptable salts thereof are also suitable for the prophylaxis and treatment of arterial vascular diseases as combination therapy with platelet aggregation inhibitors. e.g., aspirin, clopidogrel or a glycoprotein-IIb/IIIa antagonist or a thrombin receptor antagonist.

The dosage required to achieve such an effect is appropriately 0.001 to 3 mg/kg body weight, preferably 0.003 to 1.0 mg/kg body weight by intravenous route, and 0.003 to 30 mg/kg body weight, preferably 0.01 to 10 mg/kg body weight by oral route, in each case administered 1 to 4 times a day.

For this purpose, the compounds of formula (I) prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The new compounds and the physiologically acceptable salts thereof may be used therapeutically in conjunction with acetylsalicylic acid, with inhibitors of platelet aggregation such as fibrinogen receptor antagonists (e.g. abciximab, eptifibatide, tirofiban, roxifiban), with physiological activators and inhibitors of the clotting system and the recombinant analogues thereof (e.g. Protein C, TFPI, antithrombin), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel) or with a thrombin receptor antagonist (e.g. SCH-530348).

### EXPERIMENTAL SECTION

The Examples that follow are intended to illustrate the invention, without restricting its scope.

As a rule, melting points and/or IR, UV, ¹H-NMR and/or mass spectra have been obtained for the compounds prepared. Unless otherwise stated, R_{f} values were determined using ready-made silica gel 60 F₂₅₄ TLC plates (E. Merck, Darmstadt, Item no. 1.05714) without chamber saturation. The R_{f} values given under the heading Alox were determined using ready-made aluminium oxide 60 F₂₅₄ TLC plates (E. Merck, Darmstadt, Item no. 1.05713) without chamber saturation. The R_{f} values given under the heading Reversed-phase-8 (RP-8) were determined using ready-made RP-8 F₂₅₄ₛ TLC plates (E. Merck, Darmstadt, Item no. 1.15684) without chamber saturation. The ratios given for the eluants refer to units by volume of the solvents in question. For chromatographic purification silica gel made by Messrs Millipore (MATREX™, 35-70 μm) was used. Unless more detailed information is provided as to the configuration, it is not clear whether the products are pure stereoisomers or mixtures of enantiomers and diastereomers.

The following abbreviations are used in the test descriptions:

BOC tert.-butoxycarbonyl
CDI 1,1′-carbonyldiimidazole

### DIPEA N-ethyl-diisopropylamine

DMAP 4-dimethylzaminopyridine

### DMF N,N-dimethylformamide

DMSO dimethylsulfoxide
D-DTTA (+)-O,O′-di-p-toluoyl-D-tartaric acid
EDC 1-ethyl-3-(3-dimethylaminopropy)carbodiimide
EtOAc ethylacetate
sat. saturated
h hour(s)
HATU O-(7-azabenzotriazol-1-yl)-N,N,N′,N′-tetramethyluronium-hexafluorophosphate HOBT 1-hydroxy-benzotriazole
IPA isopropanol
LIHMDS lithium hexamethyldisilazide
NaHMDS sodium hexamethyldisilazide
i. vac. in vacuo
conc. concentrated
min minute(s)
MsCl methanesulfonyl chloride
MTBE methyl-tert.butylether
Me-THF 2-methyl-tetrahydrofuran

### NMM N-methyl-morpholine

Pd₂ dba₃ bis(dibenzylideneacetone)palladium(0)
n-PrOH 1-propanol
[Rh(COD)Cl]2 chloro(1,5-cyclooctadiene)rhodium
R_{f} retention factor
Rₜ retention time
rt room temperature
TBTU O-(benzotriazol-1-yl)-N,N,N′,N′-tetramethyluronium tetrafluoroborate
TEA triethylamine
TFA trifluoroacetic acid
TFFA trifluoroacetic anhydride
THF tetrahydrofuran
TsCl para-toluene sulfonic acid chloride
TsOH para-toluene sulfonic acid.
Walphos 1-[2-(2′ diphenylphosphinophenyl)ferrocenyl]ethyldiphenylphosphine

The term “thiophen-2-yl” or “thien-2-yl” denotes the group shown in the box:

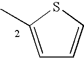

The HPLC-MS data were obtained under the following conditions:

### Method A

The mobile phase used was:

A: water with 0.15% HCOOH
B: acetonitrile

| time in min | % A | % B | flow rate in ml/min |
|---|---|---|---|
| 0.00 | 95 | 5 | 1.00 |
| 2.00 | 95 | 5 | 1.00 |
| 9.00 | 2 | 98 | 1.00 |

The stationary phase used was Zorbax StableBond C18 column; 8 μm; 50 mm×90 mm

### A) Determination of the Absolute Stereochemistry of Compound 7

The crystal structure of compound 7 was determined by direct methods. The absolute configuration was determined by refinement of the Flack parameter (Flack H D (1983), Acta Cryst. A39, 876-881).

### Crystal Data

| | |
|---|---|
| C₁₀H₁₀ClNO₄S | V = 1100.7 (4) Å³ |
| Mr = 275.70 | Z = 4 |
| Dₓ = 1.664 Mg m⁻³ | |
| a = 7.8200 (16) Å | Cu Kα |
| b = 8.7700 (18) Å | μ = 4.91 mm⁻¹ |
| c = 16.050 (3) Å | T = 100 (2) K |
| α = 90° | Prism, colourless |
| β = 90° | 0.1 × 0.1 × 0.2 mm |
| γ = 90° | |

### Data Collection

Saturn 944 CCD mounted on AFC11K diffractometer

| | |
|---|---|
| 1627 independent reflections | 1610 reflections with I > 2σ(I) |
| oscillation scans | |
| Absorption correction: | Rᵢₙₜ = 0.052 |
| empirical (using intensity measurements) | |
| 3840 measured reflections | θₘₐₓ = 63.7° |

### Refinement

Refinement on F2 w=1/[σ²(Fₒ²)+(0.0388P)²+0.2397P] where P═(Fₒ²+2F_{c}²)/3

| | |
|---|---|
| R[F² > 2σ(F²)] = 0.030 | (Δ/σ)ₘₐₓ = 0.001 |
| wR(F²) = 0.072 | Δρₘₐₓ = 0.27 e Å⁻³ |
| S = 1.08 | Δρₘᵢₙ = −0.27 e Å⁻³ |
| 1627 reflections | Extinction correction: none |
| 158 parameters | |

H atoms treated by a mixture of independent and constrained refinement Flack parameter: 0.009 (17)
Data collection: Saturn 944 CCD mounted on AFC11K/RU200 rotating anode generator; cell refinement: D*trek; data reduction: D*trek; program(s) used to solve structure: SHELXS97 program(s) used to refine structure: SHELXL97 molecular graphics: XP.

The configuration of the chiral carbon atom is S. The structure is shown in FIG. 4.

### B) Preparation of 3-amino-tetrahydro-furan-3-carboxylic acid derived intermediates

### Preparation of rac-3-amino-tetrahydro-furan-3-carboxylic acid phenethyl ester x para-toluenesulfonic acid (2)

To a solution of 3-amino-tetrahydrofuran-3-carboxylic acid 1 (100 g, 163 mmol) in 2-phenylethanol (456 mL) and toluene (400 mL) was added TsOH.H₂O (174 g, 916 mmol) under nitrogen and the mixture was heated to reflux while the water was collected via a Dean-Strak trap. At the completion of the reaction, the reaction mixture was concentrated to ⅓ of its original volume and cooled to ambient temperature. MTBE (1000 ml) was added and the mixture was stirred for 1 h. The slurry was filtered and the wet cake was washed twice with MTBE and dried to give the desired product 2 (284 g) in 92% yield.

¹HNMR (DMSO-D6, 400 MHz) δ 8.60 (br s, 3H), 7.47 (d, J=8.0 Hz, 2H), 7.20-7.50 (m, 5H), 7.11 (d, J=7.9 Hz, 2H), 4.44 (dt, J=6.6, 1.1 Hz, 2H), 3.97 (m, 1H), 3.84 (m, 3H), 2.97 (t, J=6.6 Hz, 2H), 2.32 (m, 1H), 2.30 (s, 3H), 2.09 (m, 1H)

### Preparation of rac-3-amino-tetrahydro-furan-3-carboxylic acid phenethyl ester (3)

To a 1.5 L of 5% NaHCO₃ aqueous solution was added 2 (283 g) and 1.5 L of ethyl acetate and the resulting mixture was stirred at ambient temperature for 30 min. The organic phase was removed and the aqueous phase was extracted once with 1.5 L of ethyl acetate. The combined organic phases were dried and concentrated to dryness to yield 157 g of 3 in 97% yield.

### Preparation of (S)-3-amino-tetrahydro-furan-3-carboxylic acid phenethyl ester x L-mandelic acid (4)

A mixture of 3 (157 g), MTBE (0.94 L), MeCN (0.94 L), water (0.094 L), and L-mandelic acid (152.2 g) was heated to 50-52° C. for 30 min. The mixture was cooled to 41-43° C. in an hour and 1.29 g of seed was added. After stirred for 30 min, the mixture was cooled to 0° C. and stirred for 3 h. The slurry was filtered and the wet cake was washed twice with MTBE (75 mL) and dried to give crude 4 (113.5 g) in 43.9% yield and 87% de.

The above salt was recrystallized from a mixture of MTBE (908 mL), CH₃CN (908 mL), and water (54.5 mL) to give 102.0 g of product in 89.9% yield and 98.2% de. One more crystallization from MTBE (816 mL), CH₃CN (816 mL), and water (48.9 mL) gave 94.3 g of 4 in 92.5% yield (35.0% overall yield) and 99.7% de. The enantiomerically purity was analyzed by chiral HPLC with chiralpak AD-H column, 250 cm×4.6 mm; 5 μm; 2 mL/min, 220 nm; 95% heptane/5% IPA; rₜ=9.3 min ((S)-isomer); rₜ=10.1 min, ((R)-isomer).

### Preparation of (S)-3-Amino-tetrahydro-furan-3-carboxylic acid phenethyl ester 5

A suspension of 4 (94.3 g) in 750 mL of EtOAc was added 750 mL of 5% NaHCO₃ solution and the mixture was stirred for 30 min. After the organic phase was removed, the aqueous phase was extracted once with EtOAc (750 mL). The combined organic phases were dried and concentrated to dryness to give 57.6 g of 5 in >99% yield.

### Preparation of (S)-3-[(5-chloro-thiophen-2-yl)-carbonylamino]-tetrahydro-furan-3-carboxylic acid phenethylester (6)

A mixture of 5 (20 g, 85.1 mmol), 5-chloro-thiophene-2-carboxylic acid (14.5 g, 89.4 mmol), HOBT.H₂O (13.8 g, 102 mmol), EDC.HCl (19.6 g, 102.1 mmol), and DMF (200 mL) was cooled to 10-15° C. Then TEA (17.8 mL) was added over 5 min and the mixture was warmed to ambient temperature and stirred for 2 h. EtOAc (200 mL) and water (200 mL) was added to the mixture and stirred for 20 min. The organic phase was removed and the aqueous phase was extracted once with EtOAc (200 ml). The combined organic phases were washed with 200 mL of 5% NaCl solution and dried and concentrated to give 6 (29 g) in 90% yield.

¹HNMR (CDCl₃, 400 MHz) δ 7.15-7.30 (m, 6H), 6.89 (br s, 1H), 4.42 (t, J=6.9 Hz, 2H), 3.90-4.12 (m, 4H), 2.96 (t, J=6.9 Hz, 2H), 2.53 (m, 1H), 2.29 (m, 1H).

### Preparation of (S)-3-[(5-chloro-thiophen-2-yl)-carbonylamino]-tetrahydro-furan-3-carboxylic acid (7)

A solution of 6 (40 g) in MeOH (200 mL) was cooled to 10-15° C., followed by addition of 1N NaOH (200 mL) over 5 min. The mixture was warmed to ambient temperature and stirred for 30 min. The mixture was concentrated to remove most of MeOH and MTBE (200 mL) was added and stirred the mixture for 10 min. The organic phase was removed and aqueous phase was washed once with 200 mL of MTBE. The aqueous layer was cooled to 0° C. and 3N HCl solution was added to a pH=2-3. Then 200 mL of Me-THF and 18 g of NaCl was added and the mixture was stirred for 10 min. The aqueous phase was extracted once with 100 mL of Me-THF. The combined organic phase was concentrated and 100 mL of heptane was added. The slurry was filtered and the wet cake was washed with heptane (50 mL×2) and dried to give 7 (23.3 g) in 99% yield.

Racemic compound 7 could also be prepared following the procedure described for example 1e:

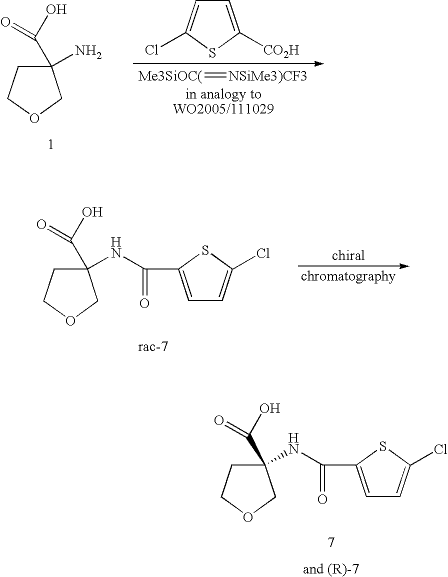

For separation of the racemic compound into its respective enantiomers, a conventional analytical HPLC system with DAICEL AD-H 250 mm×4.6 mm chiral column has been used, eluting with 0.1% Acetic Acid in Hexane (80%)/EtOH (20%) as liquid phase. At a flow rate of 1 ml/min, retention times for the enantiomers are 9.2 min and 12.5 min.

Alternatively, the separation of this racemate can be achieved on HPLC with DAICEL OJ-H chiral column, eluting with 0.1% Acetic Acid in Hexane (80%)/EtOH (20%). At a flow rate of 1 ml/min, retention times for the enantiomers are 6.05 min and 8.07 min respectively.

### Preparation of (S)-2-(5-chloro-thiophene-2-yl)-3,7-dioxa-1-aza-spiro[4.4]non-1-en-4-one (8)

50 mg (0.18 mmol) (S)-3-[(5-chloro-thiophen-2-yl)-carbonylamino]-tetrahydro-furan-3-carboxylic acid 7 were stirred for 2 h in 45 ml acetic anhydride at 85° C. The reaction mixture was evaporated down i. vac., the residue was taken up twice each in toluene and dichloromethane and completely concentrated by evaporation. The crude title compound was reacted directly without any further purification.

### Preparation of benzyl 3-tert.-butoxycarbonylamino-tetrahydro-furan-3-carboxylate (9)

A mixture of 7.0 g 3-tert.-butoxycarbonylamino-tetrahydro-furan-3-carboxylic acid, 80 ml DMF and 4.6 g K₂CO₃ was stirred for 15 min, then 3.6 ml benzylbromide was added dropwise and it was stirred for 3 days. The mixture was filtered and the liquid phase was concentrated i. vac. The residue was diluted with CH₂Cl₂, and the mixture was washed with water and saturated NaCl-solution. The organic phase was dried with Na₂SO₄ and concentrated i. vac. to yield the title compound in 58% yield.

### Preparation of benzyl 3-amino-tetrahydro-furan-3-carboxylate (10)

A mixture of 5.2 g benzyl 3-tert.-butoxycarbonylamino-tetrahydro-furan-3-carboxylate, 200 ml CH₂Cl₂ and 20 ml TFA was stirred for 3 h and concentrated i. vac. to yield the title compound in quantitative yield.

### Preparation of 3-[(5-chloro-thiophen-2-yl)-carbonylamino]-tetrahydro-furan-3-carboxylic acid benzylester 11 and separation of the enantiomers (S-12 and R-12)

1.59 g (9.8 mmol) 5-chloro-thiophene-2-carboxylic acid is dissolved in 30 ml DMF and stirred with 3.61 g (10.7 mmol) benzyl 3-amino-tetrahydro-furan-3-carboxylate and 3.46 g (10.8 mmol) TBTU and 4.3 ml (39 mmol) NMM at room temperature for 20 h. Then the mixture is evaporated down and purified by chromatography on silica gel (eluant: dichloromethane/ethanol 100:0 to 94:6).

Yield: quantitative
R_{f} value: 0.59 (silica gel; dichloromethane/ethanol=9:1)

### C₁₇H₁₆ClNO₄S (365.83)

Mass spectrum: (M+H)⁺=366/368 (chlorine isotopes)

For separation of the racemic mixture into its respective enantiomers (S-12 and R-12): a conventional HPLC system with DAICEL IA, 250 mm×4.6 mm chiral column has been used, eluting with EtOH (2%)/CHCl₃(20%)/Hexane (68%). At a flow rate of 1 ml/min, retention times for the enantiomers are 12.3 min and 20.7 min.

Alternatively, the separation of this racemate can be achieved on supercritical fluid chromatography with DAICEL IA chiral column, eluting with EtOH (15%)/CHCl₃(10%)/supercritical CO₂ (75%). At a flow rate of 70 ml/min, retention times for the enantiomers are 3.57 min and 5.13 min respectively.

### Preparation of (S)-3-[(5-chloro-thiophen-2-yl)-carbonylamino]-tetrahydro-furan-3-carboxylic acid (7)

To a solution of (S)-3-[(5-chloro-thiophen-2-yl)-carbonylamino]-tetrahydro-furan-3-carboxylic acid benzylester S-12 (5.8 g) in ethanol (100 mL) was added 1N NaOH (63 mL). The mixture stirred for 90 min and the mixture was concentrated i.vac. Cold 1N HCl was added and the mixture was stirred overnight. The precipitate was filtered and dried to give the title compound in 99% yield.

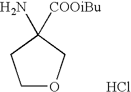

### Preparation of rac-3-Amino-tetrahydro-furan-3-carboxylic acid isobutyl ester hydrogen chloride (13)

To a mixture of amino acid 1 (190.5 g, 1.45 mol) in 2-methyl-1-propanol (1.9 L) at 0° C. was added dropwise thionyl chloride (211.6 mL, 2.0 equiv.) over 20 min. The mixture was heated to 89° C., and kept at 89° C. for 0.5 h, then heated to 108° C. and stirred at this temperature for 1.5 h. The mixture was cooled to rt and concentrated to remove most of 2-methyl-1-propanol. The residue was treated with t-butyl methyl ether (1 L) to form a suspension and stirred at rt for 0.5 h. The mixture was filtered and the cake was washed twice with t-butyl methyl ether (0.2 L×2) to provide the desired salt 13 (287 g, 1.28 mol, 88%) as a white solid.

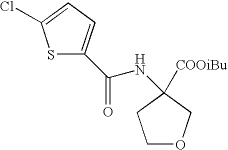

### Preparation of rac-3-[(5-Chloro-thiophene-2-carbonyl)-amino]-tetrahydro-furan-3-carboxylic acid isobutyl ester (14)

To a mixture of salt 13 (287 g, 1.28 mol), 5-chlorothiophene-2-carboxylic acid (219 g, 1.35 mol, 1.05 equiv), HOBT.H₂O (208 g, 1.54 mol, 1.2 equiv), and EDC.HCl (295 g, 1.54 mol, 1.2 equiv) in anhydrous DMF at 0° C. was added triethylamine over 10 min. The mixture was allowed to warm to rt and stirred for 3 h. EtOAc (2 L) and water (2 L) was added and the aqueous layer was removed. The EtOAc layer was further washed with water (2 L) and 5% NaCl solution (2 L) and concentrated. The residue was passed through a short silica gel plug (eluent: Hexane/EtOAc 4:1→1:1) to give the desired amide 14 (335 g, 1.01 mol, 82%) as an oil. 14: ¹HNMR (CDCl₃, 400 MHz) δ=7.47 (s, 1H), 7.40 (d, J=4.0 Hz, 1H), 6.86 (d, J=4.0 Hz, 1H), 4.26 (d, J=9.6 Hz, 1H), 3.90-4.08 (m, 4H), 2.60 (m, 1H), 2.38 (m, 1H), 1.96 (m, 1H), 0.90 (d, 4.04 (m, J=6.8 Hz, 6H);

¹³CNMR (CDCl₃, 100 MHz) δ=172.1, 163.5, 161.6, 136.7, 136.0, 128.3, 127.1, 76.2, 72.0, 67.9, 66.0, 60.5, 37.4, 27.7, 19.0; ESI-MS: m/z 332 [M+H]⁺, 685 [2M+Na]⁺.

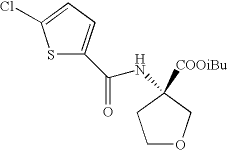

### Preparation of (S)-3-[(5-Chloro-thiophene-2-carbonyl)-amino]-tetrahydro-furan-3-carboxylic acid isobutyl ester (15)

To a 0.1 M phosphate buffer solution (pH=6.7, NaH₂PO₄.H₂O: 100.5 g, Na₂HPO₄: 80.8 g, H₂O: 6.55 L) at rt was added Alcalase (564 mL, >0.75 U/mL, 2 U/mol) and racemic ester 14 (70 g, 0.211 mol) in acetone (3.275 L). The pH value of the resulting mixture was about 7.30. The mixture was stirred at rt until the ee of the remaining ester reached 93% by chiral HPLC (about 30 h). EtOAc (2 L) was added and the aqueous layer was further extracted with EtOAc (1 L). The combined EtOAc layer was concentrated, passed through a silica gel plug (hexane: EtOAc=1:1) to give the optical enriched ester 15 (28 g, 25%, 93% ee).

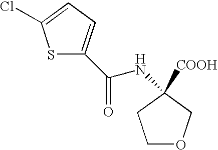

### Preparation of (S)-3-[(5-Chloro-thiophene-2-carbonyl)-amino]-tetrahydro-furan-3-carboxylic acid [(S)-7]

The optically enriched ester 15 (43.5 g, 0.131 mol) was dissolved in MeOH (400 mL). To the solution at 0° C. was added 1 N NaOH solution (400 mL) and the resulting mixture was stirred at rt for 0.5 h. The mixture was then concentrated to remove most of methanol. The resulting aqueous solution was washed once with t-butyl methyl ether (200 mL), and then neutralized with 3 N HCl at 0° C. to pH=1-2. The mixture was saturated by adding solid NaCl and extracted with Me-THF (500 mL). The Me-THF layer was dried over Na₂SO₄ and concentrated to give crude acid (S)-7 as a white solid. The solid was then recrystallized three times from water (40 mL hot water/g) to give desired acid in 99.5% ee (21 g, 48%). (S)-7: ¹HNMR (DMSO-D6, 400 MHz) δ=12.75 (s, 1H), 8.98 (s, 1H), 7.77 (d, J=4.0 Hz, 1H), 7.21 (d, J=4.0 Hz, 1H), 4.12 (d, J=9.3 Hz, 1H), 3.92 (d, J=9.3 Hz, 1H), 3.84 (t, J=7.0 Hz, 2H), 2.35 (m, 2H).

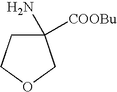

### Preparation of rac-3-Amino-tetrahydro-furan-3-carboxylic acid butyl ester (16)

To a mixture of amino acid 1 (10 g, 76 mmol) and n-butanol (100 mL) at 0° C. was added SOCl₂(18.2 g, 153 mmol, 2 equiv.) over 10 min. The mixture was heated to 110° C. over 10 min and stirred at the temperature for 4 h. After cooled to rt, the mixture was concentrated to remove n-butanol and Me-THF (100 mL) was added. To the mixture was carefully added sat. NaHCO₃ (100 mL) and the resulting mixture was stirred at rt for 30 min. The organic layer was separated and the aqueous layer was washed with Me-THF (50 mL). The combined organic phase was washed with 3% NaCl solution (50 mL) and concentrated to give the crude butyl ester 16 as an oil (14.3 g, 99.4%).

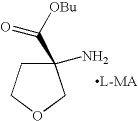

### Preparation of (S)-3-Amino-tetrahydro-furan-3-carboxylic acid butyl ester (S)-mandelic acid (17)

Butyl ester (16, 10 g, 0.053 mol) and 50 mL of MeCN was dissolved in a 250 mL reactor. (S)-Mandelic acid (4.88 g, 0.032 mol, 0.6 equiv) was added followed by addition of another 60 mL of MeCN. The mixture was heated to 70° C. and the clear solution was then cooled to 20° C. over 12 h and kept at 20° C. for 1 h. The slurry was filtered and the mother liquid was recharged back to the reactor for wash. The cake was washed with MTBE (20 mL×2) and dried under vacuum to give 7.8 g of salt with 90% de (43%). 7.5 g of the salt (90% de) was charged into a 250 mL reactor followed by MeCN (90 mL) and H₂O (0.9 mL). The mixture was heated to 70° C. and the clear solution was then cooled to 0° C. over 12 h and kept at 0° C. for 1 h. The slurry was filtered and the wet cake was washed with cold MTBE (20 mL×2, 0-5° C.) and dried under vacuum to give 17 as white crystal (6.5 g, 99.0% ee, 87%).

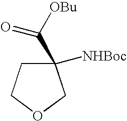

### Preparation of (S)-3-tert-Butoxycarbonylamino-tetrahydro-furan-3-carboxylic acid butyl ester (18)

Salt 17 (33.9 g, 100 mmol) was dissolved in Me-THF (150 mL) and H₂O (150 mL). To the mixture at rt was added solid NaHCO₃ (12.6 g, 150 mmol) portion wise and the mixture was stirred at rt until no gas was generated. The organic phases were separated and the aqueous layer was washed with Me-THF (50 mL). The combined Me-THF was washed with 3% NaCl (100 mL) and concentrated to 100 mL. To the mixture was added Boc₂O (21.8 g, 10 mmol) in one portion. The mixture was heated to 50° C. and stirred at the temperature for 2 h. After cooled to rt, the mixture was washed with sat. NaHCO₃ (100 mL) and brine (100 mL) and concentrated to give crude 18 as an oil (27.3 g, 95%),

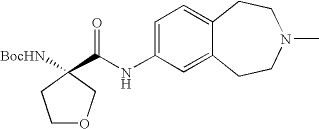

### Preparation of [(S)-3-(3-Methyl-2,3,4,5-tetrahydro-1H-benzo[d]azepin-7-ylcarbamoyl)-tetrahydro-furan-3-yl]-carbamic acid tert-butyl ester (19)

To a solution of 18 (6.1 g, 34.8 mmol) and 3-Methyl-2,3,4,5-tetrahydro-1H-benzo[d]azepin-7-ylamine (10 g, 34.8 mmol) in THF (100 mL) at −10° C. was added dropwise LHMDS (87 mL, 1.0 M in THF, 2.5 equiv.) over 10 min. The mixture was warmed up to 0° C. over 1 h and monitored by HPLC analysis. Sat. NH₄Cl (50 mL) was added at ˜0° C. to quench the reaction and the mixture was further diluted with EtOAc (100 mL). The aqueous layer was separated and extracted once with EtOAc (50 mL). The combined organic phase was washed with brine (50 mL) and concentrated. The light yellow solid was recrystallized from IPA (85 mL) at 0° C. to give 19 as a white solid (11.5 g, 85%, 98.3 A % purity).

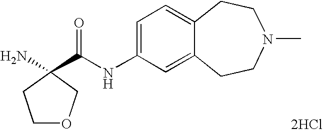

### Preparation of (S)-3-Amino-tetrahydro-furan-3-carboxylic acid (3-methyl-2,3,4,5-tetrahydro-1H-benzo[d]azepin-7-yl)-amide hydrogen chloride (20)

To a solution of 19 (2.00 g) in MeOH (10 mL) was added HCl/MeOH (˜9N, 10 mL) at ambient temperature. The resulting clear solution was stirred at ambient temperature for 1 h and then concentrated to give a white solid. MeOH (5 mL) and MTBE (30 mL) were added and the slurry was stirred at room temp for 0.5 h. The slurry was filtered, washed with MTBE (5 mL) and dried under reduced pressure to give salt 20 as a white solid (1.8 g, 96%, >99% purity).

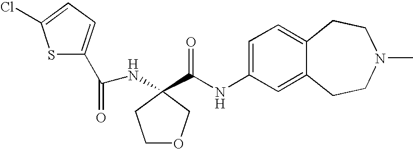

### Preparation of (S)-3-amino-tetrahydrofuran-3-carboxylic acid n-butyl ester via chemical resolution with L-mandelic acid

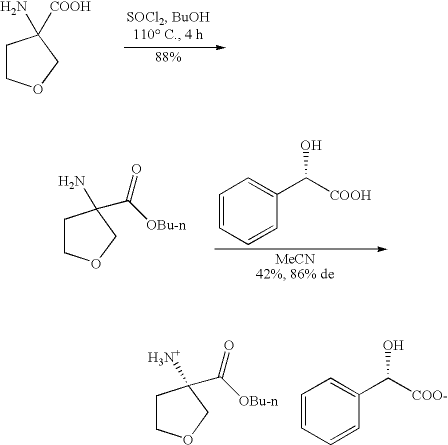

3-Aminotetrahydro-furan-3-carboxylic acid (75 g, 0.572 mol) and 750 mL n-butanol are combined into a 2 L reaction flask. The resulting suspension is cooled while stirring to about 3° C. SOCl₂ (131.6 g, 1.144 mol) is added cautiously over 25 min to the stirred suspension (exothermic). After addition is complete, the resulting mixture is heated to 110° C. over 1 h. HCl gas is generated during the course of heating and the generation of gas is vigorous when the temperature approaches over 100° C. The reaction mixture is stirred 110° C. for 4 h.

When the reaction is complete, as indicated by LC/MS, the mixture is cooled to ˜70° C. The reaction mixture is distilled to a minimum volume (˜200 mL) while controlling the temperature at 70-75° C. at 95-100 mbar. About 460 g solvents/SOCl₂ is collected. The concentrated reaction mixture is cooled to room temperature and 2-methyl-tetrahydrofuran (Me-THF)(600 mL) is added. 8% NaHCO₃ (750 mL) is added cautiously, in portions, while vigorously stirring. CO₂ is generated over the course of the addition. The resulting mixture is stirred for 15 min and then the phases are allowed to stand for 15 min. The phases are separated and then the aqueous phase is stirred with an additional 400 mL of Me-THF 15 min. After standing for 15 min, the organic layer is separated and combined with the first organic extract. 3% NaCl (375 mL) is added to the combined organic extracts and the mixture is stirred the mixture for 15 min and then allowed to stand for 15 min. The aqueous layer (˜1055 g) is removed. The organic solution is distilled to a minimum volume (˜250 mL). MeCN (1000 mL) is added in one portion and the resulting solution is distilled to a minimum volume (˜250 mL). About 800 g solvents were collected. NMR assay of the concentrated product indicated 94.0 g of the desired racemic n-butyl ester was obtained (88%).

### Chemical Resolution

MeCN (1125 mL) is added to the concentrated racemic n-butyl ester. A GC analysis should be conducted at this stage and the content of Me-THF should be controlled to be <5%. If the amount of Me-THF is >5%, the distillation of solvent and addition of 1125 mL of MeCN should be repeated.

L-Mandelic Acid (60.9 g, 0.4 mol) is added to the ester solution in one portion while stirring resulting in the formation of white solids. The mixture is heated to 70° C. and held at that temperature for 30 min, resulting in a clear solution. The solution is cooled to 20° C. over 12 h and then held at 20° C. for 1 h. The resulting slurry is filtered. The mother liquid is added back to the reaction flask to wash the flask. The filter cake is washed twice with MTBE (200 mL×2) and the resulting solid is dried at ˜50° C. under house vacuum for 3 h. (S)-3-Amino-tetrahydrofuran-3-carboxylic acid n-butyl ester L-mandelic acid salt (82.0 g, 42%) is obtained as a white solid in 86% de.

### Enrichment Procedure

The 86% de ester (82 g) is placed in a 2 L flask, under N₂ and suspended in 980 mL of CH₃CN, and 19.7 mL of water. The mixture is heated to about 70° C. to dissolve the salt, resulting in a clear solution. The solution is held at 70° C. for 30 min and then cooled 20° C. to 23° C. over 12 h. The resulting slurry is filtered and the wet filter cake is washed twice with 150 mL of MTBE. The product is dried at 50° C. under reduced pressure resulting in 66 g (81% yield) of (S)-3-amino-tetrahydrofuran-3-carboxylic acid n-butyl ester L-mandelic acid salt in 99% de.

### Preparation of (S)-3-amino-tetrahydrofuran-3-carboxylic acid n-butyl ester via chemical resolution with L-mandelic acid

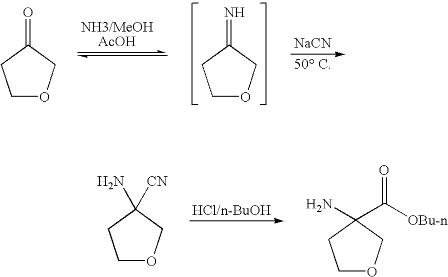

To a solution of dihydrofuran-3-one (10.0 g, 116.2 mmol) in MeOH (50 mL) was added a solution of 7 N NH₃/MeOH (33 mL) at ambient temperature. The mixture was cooled to 0° C. and 7.66 g of AcOH was added over 5 min while maintaining the temperature below 25° C. The mixture was stirred at ambient temperature for 10 min and 5.64 g of NaCN was added in one portion. The mixture was heated to 50° C., stirred for 2 h and then concentrated to remove the MeOH and ammonia. EtOAc (40 mL) was added to the concentrated mixture and then stirred for 15 min. The slurry was filtered and the wet cake was washed twice with 20 mL EtOAc. The combined filtrates, containing the desired 3-amino-tetrahydrofuran-3-carbonitrile, were concentrated and to the residue was added 20 mL of n-BuOH. The resulting mixture was cooled to 0° C. and then 100 mL of 5.5 N HCl in n-BuOH was added. The resulting mixture was stirred at ambient temperature for 12 h.

The mixture was cooled to 0° C. and 20 mL water was added. The mixture was then concentrated to remove most of the n-BuOH. Saturated NaHCO₃ was added to the residue to pH>7 and the resulting mixture was extracted twice with 200 mL of 2-methyltetrahydrofuran. The organic extracts were washed once with brine (80 mL) and then concentrated to give 18.3 g of the title compound (85% overall yield). ¹H NMR (CDCl3, ppm): 2.02 (s, 2H), 2.09-2.15 (m, 1H), 2.43-2.50 (m, 1H), 3.75-3.78 (d, J=9.04, 1H), 3.98-4.08 (m, 3H). ¹³C NMR (CDCl3, ppm) 40.80, 54.03, 67.51, 78.72, 122.63.

### Chemical Resolution

MeCN (1125 mL) is added to the concentrated racemic n-butyl ester. A GC analysis should be conducted at this stage and the content of Me-THF should be controlled to be <5%. If the amount of Me-THF is >5%, the distillation of solvent and addition of 1125 mL of MeCN should be repeated.

L-Mandelic Acid (60.9 g, 0.4 mol) is added to the ester solution in one portion while stirring resulting in the formation of white solids. The mixture is heated to 70° C. and held at that temperature for 30 min, resulting in a clear solution. The solution is cooled to 20° C. over 12 h and then held at 20° C. for 1 h. The resulting slurry is filtered. The mother liquid is added back to the reaction flask to wash the flask. The filter cake is washed twice with MTBE (200 mL×2) and the resulting solid is dried at ˜50° C. under house vacuum for 3 h. (S)-3-Amino-tetrahydrofuran-3-carboxylic acid n-butyl ester L-mandelic acid salt (82.0 g, 42%) is obtained as a white solid in 86% de.

### Enrichment Procedure

The 86% de ester (82 g) is placed in a 2 L flask, under N₂ and suspended in 980 mL of CH₃CN, and 19.7 mL of water. The mixture is heated to about 70° C. to dissolve the salt, resulting in a clear solution. The solution is held at 70° C. for 30 min and then cooled 20° C. to 23° C. over 12 h. The resulting slurry is filtered and the wet filter cake is washed twice with 150 mL of MTBE. The product is dried at 50° C. under reduced pressure resulting in 66 g (81% yield) of (S)-3-amino-tetrahydrofuran-3-carboxylic acid n-butyl ester L-mandelic acid salt in 99% de.

Alternatively, various alcohols (ROH) are used in as shown in the Table below.

| Entry | Alcohol (ROH) | Conditions | Yield of esters |
|---|---|---|---|
| 1 | PhCH₂CH₂OH | H₂SO₄, 90° C., 6 h | 10% |
| 2 | PhCH₂CH₂OH | H₂SO₄, Cul, 90° C., 6 h | 9% |
| 3 | PhCH₂CH₂OH | TsOH, 90° C., 6 h | ~39% |
| 4 | PhCH₂CH₂OH | TsOH, CuI, 90° C., 6 h | ~39% |
| 5 | PhCH₂CH₂OH | Conc. HCl, 75° C., 3 h | 15% |
| 6 | PhCH₂CH₂OH | 2 N HCl, rt, 18 h | trace |
| 7 | PhCH₂CH₂OH | 6 N HCl, rt, 2 h | ~30% |
| 8 | MeOH | 9N HCl, rt, 18 h | Product on LC |
| | | | yield N/A |
| 9 | PhCH₂CH₂OH | ~7 N HCl, rt, 18 h | ~33% |
| 10 | PhCH₂CH₂CH₂OH | ~6 N HCl, rt, 18 h | ~9% |
| 11 | nBuOH | ~2 N HCl, 50° C., 2 h | ~15% |
| 12 | nBuOH | ~9 N HCl, rt, 18 h | ~70% |
| 13 | nBuOH | ~9 N HCl, rt, 18 h | ~73% |
| 14 | Iso-BuOH | ~9 N HCl, rt, 18 h | ~69% |

### Preparation of (S)-3-[(5-Chloro-thiophene-2-carbonyl)-amino]-tetrahydro-furan-3-carboxylic acid ((S)-3,5-dimethyl-2,3,4,5-tetrahydro-1H-3-5 benzazepin-7-yl)-amide

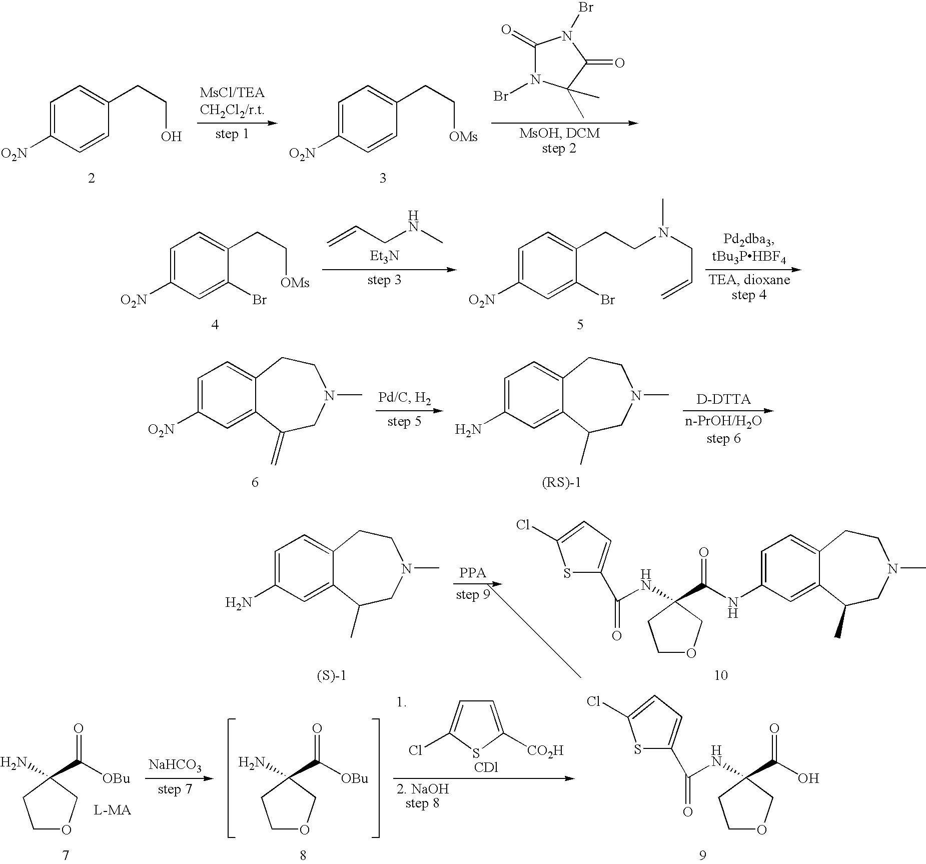

### Resolution of (RS)-1 to Prepare (S)-1:

To a 2 L jacked-flask with condenser and mechanical stirrer was charged crude (RS)-1 (84 g), nPrOH (840 mL), and D-DTTA (122 g). The mixture was heated to 60° C. and then water (252 mL) was added. The mixture was then heated to reflux to become a clear solution. After stirring at reflux for ˜0.5 h, the mixture was cooled to ambient temperature over 2 h, and further cooled to 0° C. over 0.5 h and kept at 0° C. for 1 h. The slurry was filtered and washed with cold solvent to give the salt (101 g) in 39% yield and 95% de.

The salt was then recrystallized from nPrOH/water (800 mL/200 mL) to give the (S)-1 (95 g) in >99% de and 37% overall yield.

Preparation of 4 from 2

To a 2 L reaction was charged 2-(4-nitrophenyl)ethanol 2 (150 g, 0.882 mol), dichlormethane (1 L) and methanesulfonyl chloride (75.8 mL, 0.971 mol, 1.1 eq) at 20-25° C. The mixture was cooled to −10° C., and then triethylamine (107 g, 1.06 mol, 1.2 eq) was added slowly over 2 h while maintaining the internal temperature <5° C. The mixture was then warmed to 25° C. and stirred at this temperature for 1.5 h. Additional MsCl (4 mL, 0.05 eq) was added in one portion. The mixture was further stirred at 20-25° C. for 1 h. 1 N HCl (800 mL) was added and the mixture was stirred at 20-25° C. for 10 min. The organic layer was separated and the aqueous layer was discarded. The organic phase was washed with 5% NaCl solution (500 mL) and concentrated to about 800 mL. To the solution was charged methanesulfonic acid (285 mL, 4.41 mol, 5 eq) in one portion, followed by addition of 1,3-dibromo-5,5-hydantoin (151 g, 0.53 mol, 0.6 eq) in several portions over 5 min. The mixture was stirred at 25-32° C. for 2 h. Additional 1,3-dibromo-5,5-hydantoin (25 g, 0.09 mol, 0.1 eq) was added and the mixture was further stirred at 25-30° C. for 4 h. The reaction mixture was cooled to 0° C. and water (800 mL) was cautiously added over 30 min while controlling the temperature <35° C. The organic phase was separated and washed sequentially with 10% Na₂S₂O₃ (500 mL), 3% NaHCO₃ (500 mL), 5% NaCl (500 mL), and concentrated to give crude 4 as an oil, which was directly used in the next step.

### Preparation of 5 Via Amination:

To a solution of TEA (180 mL) and methylallylamine (96.5 g) in DMF (250 mL) at 5° C. was added dropwise a solution of crude 4 in DMF (200 mL) over 1 h while controlling the reaction temperature <15° C. The mixture was kept stirring at 15-18° C. for 2 h, then quenched with 3% NaHCO₃ (1 L) and EtOAc (1 L). The organic phase was separated and the aqueous phase was further extracted once with EtOAc (500 mL). The combined organic phase was washed with brine, concentrated to about 1 L, and cooled to 0-5° C. HCl (˜64.8 g) gas was bubbled through the solution to form a slurry. The slurry was filtered and the wet cake was washed with EtOAc (200 mL), dried at 20-25° C. with house vacuum over 12 h to give the desired HCL salt of 5 as a white solid (241 g, 0.75 mol, 85% overall yield from 2). ¹HNMR (400 MHz, CDCl₃) δ 8.40 (d, J=2.3 Hz, 1H), 8.10 (dd, J=8.4, 2.3 Hz, 1H), 7.42 (d, J=8.4 Hz, 1H), 5.83 (m, 1H), 5.17 (m, 2H), 3.09 (d, J=6.4 Hz, 2H), 3.01 (m, 2H), 2.65 (m, 2H), 2.34 (s, 3H)

### Synthesis of Compound 6 via a Heck Reaction

To a 2 L 3-necked flask equipped with mechanical stirrer and thermometer at room temperature was charged aryl amine (85 g, 256 mmol), dioxane (640 mL) and triethylamine (52 g, 511 mol, 2 eqiuv.) under argon. The mixture was degassed with argon for 15 min then Pd₂ dba₃ (11.7 g) and PtBu₃.HBF₄ (7.4 g) were added under argon. The whole mixture was further degassed at room temperature for 5 min, and then heated to 100° C. and stirred at this temperature under argon for 1.5 h. The mixture was cooled to ˜50° C. and distilled under house vacuum to remove most of dioxane, and then EtOAc (0.8 L) and 3% NaHCO₃ (0.5 L) solution were added. The mixture was filtered through a pad of diatomaceous earth to remove some precipitated palladium species. The organic phase was separated and the aqueous phase was extracted once with EtOAc. The combined organic phase was washed with brine (0.4 L) and concentrated to about 0.5 L. 5-6 M HCl in IPA (56 mL) was added at 0° C. and the resulting slurry was filtered. The wet cake was washed and dried to give the HCL salt of 6 as a yellowish solid (66 g, 84% yield, 94% purity). ¹HNMR (400 MHz, CDCl₃) δ 8.10 (d, J=2.4 Hz, 1H), 8.03 (dd, J=8.2, 2.4 Hz, 1H), 7.24 (d, J=8.3 Hz, 1H), 5.37 (d, J=1.1 Hz, 1H), 5.35 (d, J=1.0 Hz, 1H), 3.34 (s, 2H), 2.98 (m, 2H), 2.81 (m, 2H), 2.43 (s, 3H).

Preparation of 9 by using CDI as Coupling Reagent:

To a suspension of 5-chloro-thiophenecarboxylic acid (25.7 g) in 100 mL of EtOAc was added CDI (24.85 g) in one portion at 20-25° C. over 10 min while gas was generated vigorously. The mixture was heated to reflux for 30 min and then cooled to room temperature. DMAP (1.44 g) was added followed by the amino acid butyl ester 8 (22 g) in EtOAc (150 mL) in one portion. The mixture was reflux for 20 h and cooled to 0-5° C. 3 N HCl (150 mL) was added and the resulting mixture was stirred at room temperature for 10 min. The organic phase was separated, washed with 5% NaHCO₃ (150 mL) and 5% NaCl solution (50 mL). The organic phase was concentrated to ˜50 mL and MeOH (150 mL) was added. The mixture was further concentrated to ˜100 mL.

To the above solution was added 2 N NaOH (90 mL, 1.5 equiv) and the mixture was stirred at room temperature for 2 h. The mixture was distilled to remove most of the MeOH and then cooled to 0° C. 12 N HCl (˜20 mL) was added dropwise to adjust the pH to 1˜2 while maintaining the internal temperature <30° C. The resulting mixture was extracted with Me-THF (200 mL), The organic layer was separated, washed with 5% NaCl (150 mL), and concentrated to ˜100 mL. To the residue was added heptane (100 mL) to form a slurry, which was filtered to give the desired product 9 (31.3 g, 96% yield, >98% purity) as a white solid.

### Preparation of (S)-1 Via Asymmetric Hydrogenation of 6. HCl:

[Rh(COD)Cl]₂ (4.9 mg, 0.01 mmol) and Walphos (13.16 mg, 0.02 mmol) were stirred in 2 mL degassed MeOH at for 10 min before being transferred to a solution of compound 6 (127 mg, 0.5 mmol) in MeOH (2 mL). The mixture was stirred at room temperature under 100 psi H₂ for 12 h. HPLC showed complete reduction of the double bond. To the mixture was added 10% Pd/C (10 mg). The mixture was further stirred at room temperature under 100 psi H₂ for 2 h, then filtered, and concentrated to give pure compound (S)-1. Chiral HPLC showed 79% ee.

### Preparation of 10:

Carboxylic acid 9 (3.04 g, 11.0 mmol, 1.05 eq) was dissolved in dry THF (50 mL) at room temperature followed by sequential addition of TEA (5.11 mL, 36.8 mmol, 3.5 eq) and propylphosphonic acid anhydride in EtOAc (50% w/w, 7.0 g, 6.48 mL, 11.0 mmol, 1.05 eq). The mixture was stirred at room temperature for 10 min. To the mixture was added aniline (S)-1 (2.0 g, 10.5 mmol, 1.0 eq) in THF (10 mL) at room temperature. The resulting mixture was stirred at reflux for 2 h. LC showed 88 A % conversion of product along with 12% mixed anhydride. The mixture was quenched by adding 40 mL saturated NaHCO₃ solution and then was concentrated to remove most of THF. To the residue was added Me-THF (40 mL). The organic phase was separated and the aqueous layer was further washed with Me-THF (20 mL). The combined Me-THF extracts were washed with brined, dried over Na₂SO₄, concentrated, and purified by column chromatography (EtOAc/EtOH/Et₃N=2:1:0.06) to give the desired product 10 (4.13 g, 9.2 mmol, 83%) as a white solid. ¹HNMR (400 MHz, CCl₃) δ 8.41 (s, 1H), 7.42 (s, 1H), 7.35 (d, J=4.0 Hz, 1H), 7.27-7.33 (m, 2H), 7.06 (d, J=8.0 Hz, 1H), 6.91 (d, J=4.0 Hz, 1H), 4.34 (d, J=9.4 Hz, 1H), 4.19-4.31 (m, 2H), 4.15 (d, J=9.4 Hz, 1H), 3.19 (br, 1H), 3.03 (br, 1H), 2.75-2.95 (m, 3H), 2.71 (d, J=12.3 Hz, 1H), 2.48 (m, 1H), 2.37 (s, 3H), 2.20-2.33 (br, 2H), 1.38 (d, J=7.2 Hz, 3H); ¹³CNMR (100 MHz, CCl₃) δ 18.5, 35.4, 35.9, 47.6, 57.3, 64.3, 65.1, 68.1, 72.6, 117.7, 127.2, 127.7, 129.9, 135.7, 136.1, 137.4, 138.0, 146.2, 160.6, 171.7; ESI MS: 448 [M+H].

### Preparation of (S)-3,5-Dimethyl-2,3,4,5-tetrahydro-1H-3-benzazepin-7-ylamine

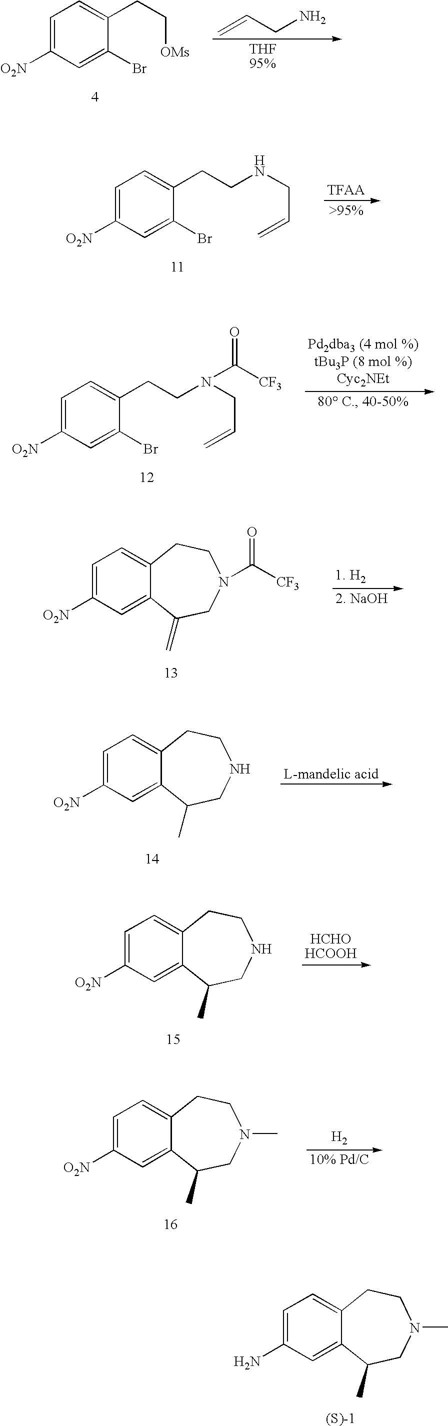

### Preparation of 11 and 12:

To a solution of 4 (120 g, 370 mmol) in dry DMF (750 mL) at ambient temperature is added allylamine (127 g, 2220 mmol). The reaction mixture is stirred at room temperature for 1 h. HPLC showed completion of the reaction. EtOAc (500 mL) and water (500 mL) were added to the reaction mixture. The organic layer was separated and the aqueous layer was extracted with EtOAc (200 mL). The combined organic layer was washed with brine (2×250 mL) and concentrated to give the desired product 11 (103 g) in 97% yield.

To a solution of 11 in CH₂Cl₂ (1 L) at 0° C. was added TEA (2 eq) and TFAA (1.2 eq) over 0.5 h. The mixture was allowed to warm to room temperature and was stirred for 3 h. Water (0.5 L) was added to quench the reaction and the resulting mixture was further stirred at room temperature for 10 min. The organic layer was separated and the aqueous layer was extracted with CH₂Cl₂ (200 mL). The combined CH₂Cl₂ was washed with water (0.5 L), brine (0.5 L), then concentrated to give the desired product as a brown solid (95% yield). ¹HNMR (400 MHz, CDCl₃) δ 8.45 (m, 1H), 8.14 (m, 1H), [7.44 (d, J=8.4 Hz, major); 7.40 (d, J=8.4 Hz, minor); 1H], [5.82 (m, minor), 5.68 (m, major); 1H], 5.20-5.32 (m, 2H), 3.92 (d, J=5.5 Hz, 1H), 3.62 (t, J=7.8 Hz, 2H), 3.18 (m, 2H).

### Preparation of 13:

To a mixture of aryl bromide 12 (2.6 g, 6.82 mmol, 1.0 eq), Pd₂ dba₃ (250 mg, 0.273 mmol, 0.04 eq), and N-methyldicyclohexylamine (2.00 g, 10.23 mmol, 1.5 eq) in anhydrous dioxane (60 mL, 0.1 M) under argon was added 10% (w/w) t-Bu₃P in hexane (1.62 mL, 0.55 mmol, 0.08 eq). The mixture was stirred under argon at 80° C. for 2 h and then cooled to room temperature, quenched by adding water (40 mL) and EtOAc (40 mL). The organic phase was separated and the aqueous layer was washed once with EtOAc (40 mL). The combined EtOAc was washed with water (40 mL) and brine (40 mL), dried over Na₂SO₄, and purified by column chromatography to give 13 (1.1 g, 3.77 mmol, 54%) as a yellow solid along with 5-10% 8-membered ring by-product. (two atropisomers):

¹HNMR (400 MHz, CDCl₃) δ 8.19 (m, 1H), 8.11 (m, 1H), 7.33 (m, 1H), 5.40-5.60 (m, 2H), 4.48 (d, J=15.9 Hz, 2H), 3.89 (m, 2H), 3.15 (m, 2H); ¹³CNMR (100 MHz, CDCl₃) δ 33.1, 35.22, 44.8, 46.2, 47.7, 50.4, 50.7, 114.9, 117.7, 118.7, 120.5, 122.3, 123.1, 123.4, 123.6, 123.7, 130.5, 130.6, 141.0, 141.4, 142.7, 143.2, 143.3, 143.9, 147.4.

### Preparation of 14:

To a 300 mL autoclave was added Wilkinson catalyst RhCl(PPh₃)₃ (1.78 g, 1.92 mmol, 0.04 eq) followed by a solution of compound 18 (14.4 g, 48.0 mmol, 1.0 eq) in THF (100 mL). The mixture was stirred at room temperature under 30-40 psi H₂ for 12 h. LC showed complete conversion and no trace of over-reduced by-product was observed. Note: Around 10% of 8-membered ring by-product, which was generated from Heck reaction, was seen. The mixture was concentrated and purified by column chromatography (hexane→hexane/EtOAc=3/1) to give the desired hydrogenation product as a white solid. To a solution of the hydrogenation product in THF (80 mL) at 0° C. was added a solution of NaOH (1.98 g, 49.6 mmol) in water (20 mL). The mixture was stirred at room temperature for 2 h. LC showed completion of hydrolysis. The mixture was concentrated and the residue was treated with Me-THF (250 mL) and water (100 mL). The Me-THF layer was separated and the aqueous layer was washed with Me-THF (100 mL). The combined Me-THF was washed with brine (100 mL), dried over Na₂SO₄, and concentrated to give the crude product (8.9 g, 43.2 mmol, 90% yield) as a yellow solid.

### Preparation of 15:

A mixture of racemic amine 14 (8.0 g, 38.8 mmol, 1 eq) and L-madelic acid (4.43 g, 29.1 mmol, 0.75 eq) in acetone (90 mL) and water (9 mL) was heated to reflux to become a transparent solution. The mixture was cooled to 0° C. over 6 h while stirring. The resulting slurry was filtered to give enantiomerically enriched salt with 58% ee (7.0 g, 50.4% yield). The salt was further crystallized from ethanol-water for 5 consecutive times to give the salt (2.5 g, 18%) with 97.0% ee. The salt was then treated with 2 N NaOH (20 mL) and Me-THF (50 mL). The Me-THF layer was separated and the aqueous layer was extracted with Me-THF. The combined Me-THF layer was washed with brine (20 mL), dried over Na₂SO₄, and concentrated to give compound 15 as a yellow crystalline solid (1.44 g, 7.0 mmol, ee: 97.0%, 18% yield).

### Preparation of 16 and (S)-1

The intermediate 15 (1.44 g, 7.0 mmol) was dissolved in 10 mL of HCOOH. To the mixture at room temperature was added 37% HCHO (0.81 g, 37% w/w, 10.5 mmol). The mixture was stirred at 90° C. for 3 h, and concentrated to give a yellow oily product. The residue was diluted with Me-THF (50 mL) and 2 N NaOH (20 mL) and stirred at room temperature for 10 min. The aqueous layer was further washed with Me-THF and the combined Me-THF was washed with brine and concentrated to give methylated compound 16.

The above residue (1.8 g, 8.2 mmol, 98.2% ee) was dissolved in MeOH (20 mL) and 10% Pd/C (200 mg) was added. The mixture was stirred at room temperature under 100 psi H₂ for 12 h, filtered to remove Pd/C, and concentrated. The residue was purified by column chromatography [EtOAc(Et₃N 0.06 v/v)/MeOH=100/0→50/50 as eluent) to give the aniline compound (S)-1 (1.57 g, 8.2 mmol, 100%, 98.2% ee, 98.5 A % HPLC purity) as a brown oil. ¹HNMR (400 MHz, CD₃OD) δ 6.82 (d, J=7.9 Hz, 1H), 6.61 (s, 1H), 6.49 (dd, J=7.8, 2.2 Hz, 1H), 3.07 (m, 1H), 2.93 (m, 1H), 2.60-2.85 (m, 3H), 2.30 (s, 3H), 2.18 (br, 2H), 1.32 (d, J=7.3 Hz, 3H); ¹³CNMR (100 MHz, CD₃OD) δ 19.2, 35.6, 47.9, 59.2, 66.0, 114.4, 114.6, 131.0, 132.3, 146.9, 147.0; ESI MS: 191 [M+H].

### Preparation of (S)-3,5-Dimethyl-2,3,4,5-tetrahydro-1H-3-benzazepin-7-ylamine

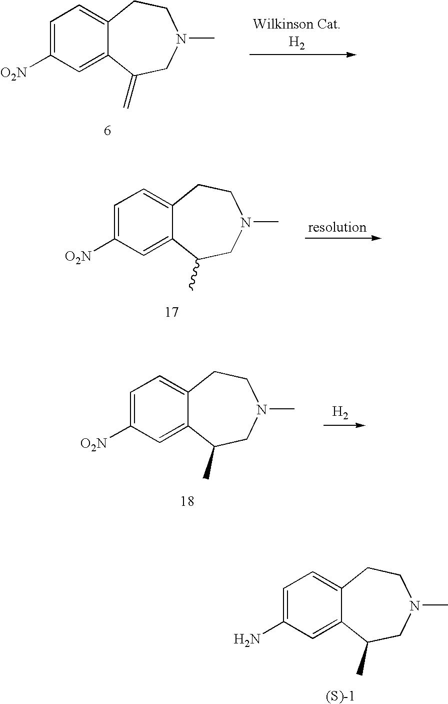

### Preparation of 17:

To a solution of olefin 6 (200 mg, 0.92 mmol) in THF (2 mL) was added RhCl(PPh₃)₃ (34 mg, 0.037 mmol, 0.04 eq). The mixture was stirred at room temperature under 30-40 psi H₂ for 12 h, then concentrated and purified by column chromatography to give the desired amine 17 as a thick oil. 17: ¹HNMR (400 MHz, CDCl₃) δ 8.05 (d, J=2.2 Hz, 1H), 8.00 (dd, J=8.2, 2.3 Hz, 1H), 7.24 (d, J=8.2 Hz, 1H), 3.29 (m, 1H), 3.22 (m, 1H), 2.80-3.10 (m, 2H), 2.89 (d, J=12.3 Hz, 1H), 2.37 (s, 3H), 2.15-2.30 (m, 2H), 1.44 (d, J=7.2 Hz, 3H).

### Preparation of 18:

Same procedure was used for the resolution of (RS)-1 by using DTTA.

### C) Examples

### Example 1

### (R)- and (S)-3-[(5-bromo-thiophen-2-yl)-carbonylamino]-N-(3-methyl-2,3,4,5-tetrahydro-1H-benzo[d]azepin-7-yl)-tetrahydrofuran-3-carboxylic acid amide

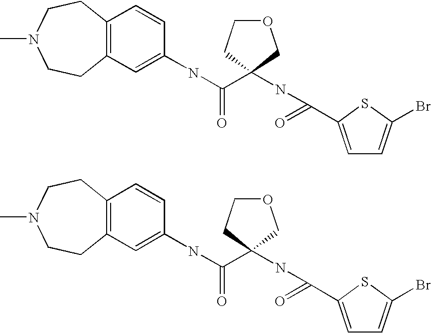

### (a) 7-nitro-2,3,4,5-tetrahydro-1H-benzo[d][azepine

8.4 g (29.0 mmol) 3-trifluoroacetyl-7-nitro-2,3,4,5-tetrahydro-1H-benzo[d]azepine are suspended under a nitrogen atmosphere in 80 ml of methanol and combined with 5 ml NaOH solution (50%) and stirred for 2 h at 70° C.

The methanol is distilled off using the rotary evaporator, the residue is combined with water and extracted with tert.-butylethylether. The organic phase is washed with NaOH solution (50%) and sat. sodium chloride solution, dried on sodium sulphate and evaporated to dryness i. vac.

### Yield: 5.1 g (91%)

R_{f} value: 0.28 (aluminium oxide; dichloromethane/ethanol=95:5)

### C₁₀H₁₂N₂O₂ (192.22)

Mass spectrum: (M+H)⁺=193

### (b) 3-methyl-7-nitro-2,3,4,5-tetrahydro-1H-benzo[d]azepine

5.0 g (26.0 mmol) 7-nitro-2,3,4,5-tetrahydro-1H-benzo[d][azepine are mixed in 9.8 ml formic acid with 15.5 ml formalin solution in water (37%) at room temperature, and stirred overnight at 70° C. The reaction mixture is made alkaline with NaOH solution (50%) while cooling with an ice bath and extracted with tert.-butylmethylether. The organic phase is dried on sodium sulphate and evaporated to dryness i. vac.

### Yield: 4.8 g (90%)

R_{f} value: 0.65 (aluminium oxide; dichloromethane/ethanol=95:5)

### C₁₁H₁₄N₂O₂ (206.24)

Mass spectrum: (M+H)⁺=207

### (c) 3-methyl-2,3,4,5-tetrahydro-1H-benzo[d]azepin-7-ylamine

4.8 g (23.2 mmol) 3-methyl-7-nitro-2,3,4,5-tetrahydro-1H-benzo[d]azepine are dissolved in 45 ml of methanol and combined with 400 mg Pd/C 10%. The mixture is hydrogenated in a Parr apparatus at room temperature at 3 bar hydrogen pressure for 5 hours. Then the catalyst is filtered off and the filtrate is evaporated down i. vac.

### Yield: 3.9 g (96%)

R_{f} value: 0.36 (aluminium oxide; dichloromethane/ethanol=98:2)

### C₁₁H₁₆N₂ (176.26)

Mass spectrum: (M+H)⁺=177

### (d) 3-amino-tetrahydro-furan-3-carboxylic acid-hydrochloride

3.5 g (15.1 mmol) 3-tert.-butoxycarbonylamino-tetrahydro-furan-3-carboxylic acid are dissolved in 150 ml of 1-molar hydrochloric acid and stirred for 1 h at room temperature. Then the reaction mixture is lyophilised.

### Yield: 2.5 g (100%)

### C₅H₉NO₃*HCl (167.59)

Mass spectrum: (M+H)⁺=132

### (e) 3-[(5-bromo-thiophen-2-yl)-carbonylamino]-tetrahydro-furan-3-carboxylic acid

3.1 g (14.9 mmol) 5-bromo-thiophene-2-carboxylic acid in 50 ml dichloromethane are combined with 5.4 ml (74.6 mmol) thionyl chloride with stirring at room temperature and stirred for 3.5 h at reflux temperature. Then the reaction mixture is evaporated to dryness.

2.5 g (14.9 mmol) 3-amino-tetrahydro-furan-3-carboxylic acid-hydrochloride are dissolved in 2.0 ml (14.9 mmol) TEA and 150 ml acetonitrile and combined with 5.9 ml (22.4 mmol) N,O-bis-(trimethylsilyl)-trifluoro-acetamide with stirring and refluxed for 4 h with stirring. The reaction mixture is combined with 4.1 ml (29.8 mmol) TEA and the solution of the prepared acid chloride in 50 ml acetonitrile, stirred for 15 min at reflux temperature and then cooled slowly to room temperature. Then the mixture is evaporated to dryness i. vac., the residue is combined with water and 2-molar sodium carbonate solution and washed with diethyl ether. The aqueous phase is adjusted to pH 1 with 20 ml conc. hydrochloric acid, the precipitate is suction filtered and dried at 50° C. in the vacuum drying cupboard.

### Yield: 3.6 g (75%)

### C₁₀H₁₀BrNO₄S (320.16)

Mass spectrum: (M−H)⁻=318/320 (bromine isotopes)

### (f) 3-[(5-bromo-thiophen-2-yl)-carbonylamino]-N-(3-methyl-2,3,4,5-tetrahydro-1H-benzo[d]azepin-7-yl)-tetrahydrofuran-3-carboxylic acid amide

700.0 mg (2.19 mmol) 3-[(5-bromo-thiophen-2-yl)-carbonylamino]-tetrahydro-furan-3-carboxylic acid are combined with 890.0 mg (2.34 mmol) HATU and 601.0 μl (5.47 mmol) NMM in 10 ml DMF with stirring at room temperature and stirred for 10 min. Then 385.0 mg (2.19 mmol) 3-methyl-2,3,4,5-tetrahydro-1H-benzo[d]azepin-7-ylamine are added and the mixture is stirred overnight at 65° C. The reaction mixture is combined with water and sat. sodium hydrogen carbonate solution, the precipitate is filtered off and purified by chromatography on aluminium oxide (eluant: dichloromethane/ethanol 100:0 to 98:2).

### Yield: 850.0 mg (81%)

R_{f} value: 0.62 (aluminium oxide; dichloromethane/ethanol=95:5)

### C₂₁H₂₄BrN₃O₃S (478.40)

Mass spectrum: (M+H)⁺=478/480 (bromine isotopes)

For separation of the racemic mixture into its respective enantiomers, a conventional HPLC system with DAICEL AD-H 250 mm×4.6 mm chiral column has been used, eluting with (0.2% Diethylamine in Hexane)/isopropanol 70/30 as liquid phase. At a flow rate of 1 ml/min, retention times for the enantiomers are 13.6 min and 16.4 min.

### Example 2

### (R)- and (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

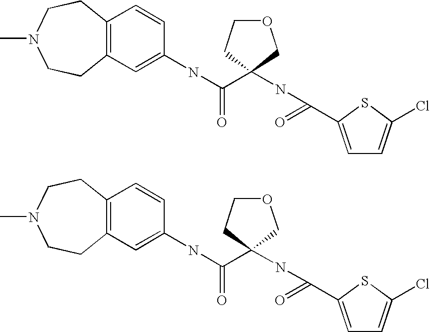

### (a) benzyl 3-[(5-chloro-thiophen-2-yl)-carbonylamino]-tetrahydro-furan-3-carboxylate

1.59 g (9.8 mmol) 5-chloro-thiophene-2-carboxylic acid is dissolved in 30 ml DMF and stirred with 3.61 g (10.7 mmol) benzyl 3-amino-tetrahydro-furan-3-carboxylate and 3.46 g (10.8 mmol) TBTU and 4.3 ml (39 mmol) NMM at room temperature for 20 h. Then the mixture is evaporated down and purified by chromatography on silica gel (eluant: dichloromethane/ethanol 100:0 to 94:6).

Yield: quantitative
R_{f} value: 0.59 (silica gel; dichloromethane/ethanol=9:1)

### C₁₇H₁₆ClNO₄S (365.83)

Mass spectrum: (M+H)⁺=366/368 (chlorine isotopes)

### (b) 3-[(5-chloro-thiophen-2-yl)-carbonylamino]-tetrahydro-furan-3-carboxylic acid

3.6 g (9.8 mmol) benzyl 3-[(5-chloro-thiophen-2-yl)-carbonylamino]-tetrahydro-furan-3-carboxylate are dissolved in 60 ml of ethanol and combined with 39.1 ml (39.1 mmol) 1-molar aqueous sodium hydroxide solution and stirred for 6 h at room temperature. After evaporation i. vac. the residue is combined with 1-molar aqueous hydrochloric acid while cooling with an ice bath, the precipitate is suction filtered and dried at 60° C. in the vacuum drying cupboard.

### Yield: 2.5 g (91%)

R_{f} value: 0.13 (silica gel; dichloromethane/ethanol 9:1)

### C₁₀H₁₀ClNO₄S (275.71)

Mass spectrum: (M−H)⁻=274/276 (chlorine isotopes)

### (c) 3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

Prepared analogously to Example 2(a) from 3-[(5-chloro-thiophen-2-yl)-carbonylamino]-tetrahydro-furan-3-carboxylic acid and 3-methyl-2,3,4,5-tetrahydro-1H-benzo[d]azepin-7-ylamine with TBTU and TEA in THF at room temperature with subsequent purification by chromatography with aluminium oxide (eluant: dichloromethane/ethanol 100:0 to 97:3).

### Yield: 67%

R_{f} value: 0.63 (aluminium oxide; dichloromethane/ethanol=95:5)

### C₂₁H₂₄ClN₃O₃S (433.95)

Mass spectrum: (M+H)⁺=434/436 (chlorine isotopes)

For separation of the racemic mixture into its respective enantiomers, a conventional analytical HPLC system with DAICEL IA 250 mm×4.6 mm chiral column has been used, eluting with EtOH as liquid phase. At a flow rate of 0.5 ml/min, retention times for the enantiomers are 13.10 min and 16.30 min.

Alternatively, for separation of the racemic mixture into its respective enantiomers, a conventional HPLC system with DAICEL AD-H 250 mm×4.6 mm chiral column has been used, eluting with (0.2% Cyclohexylamine in Hexane)/isopropanol 70/30 as liquid phase. At a flow rate of 1 ml/min, retention times for the enantiomers are 12.8 min and 15.2 min.

### d) (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide was also prepared according to the following procedure

To a mixture of 1.08 g (6.13 mmol) 7 and 1.86 g (6.75 mmol) 3-methyl-2,3,4,5-tetrahydro-1H-benzo[d]azepin-7-ylamine in 50 ml THF, was added 2.98 ml TEA and 4.7 ml 1-propylphosphonic acid cyclic anhydride (50% in EtOAc). The mixture was refluxed for 2 h, the solvents were removed i. vac. and the crude mixture was purified by chromatography (Method A) to yield the title compound in 89%.

or to the following procedure:

To a solution of 5-chloro-thiophene-2-carboxylic acid in MeCN (0.1 g, 0.615 mmol) was added TsCl (0.106 g, 0.554 mmol) in one portion. After the mixture was cooled to 0° C., NMM (0.38 mL, 310 mg, 3.08 mmol, 5 equiv.) was added slowly and the mixture was warmed to room temperature and stirred for 3 h, then heated to 50° C. for 0.5 h. The reaction was monitored by HPLC for the disappearance of TsCl (<1% by area). Salt 20 (0.141 g, 0.388 mmol, KF ˜1%) was added and the reaction mixture was stirred at ambient temperature for 2 h and then concentrated to remove MeCN. EtOAc (50 mL) and sat. NaHCO₃ (50 mL) was added and the mixture was stirred at rt for 15 min. The organic phase was separated, washed with sat. NaHCO₃ solution (50 mL) and brine (50 mL), and concentrated to give the desired example (S)-2 as a white solid (75% based on HPLC assay).

The following solubility and solid state characteristics of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide and of its anhydrous crystalline form are relevant to the present invention.

### Solubility properties of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

### Solubility and Dissolution Rate in Aqueous Media

The table below shows the values of solubility of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide in different aqueous media.

| | solubiliy | pH of the |
|---|---|---|
| medium | [mg/ml] | saturated solution |
| water | 0.28 | 8.6 |
| 0.1 N HCl | >10 | 1.0 |
| pH 2.2 | >10 | 2.4 |
| pH 4.0 | >10 | 4.3 |
| pH 6.0 | >10 | 6.3 |
| pH 7.4 | 1.8 | 7.5 |
| 0.01 N NaOH | 0.046 | 11.8 |

The table below shows the values of intrinsic dissolution rate of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide in aqueous media. The intrinsic dissolution rate was determined in aqueous media covering a range of pH 1.1-7.4 using the rotating disc method which maintains a constant surface area. 5 mg of drug substance was compressed to form a disc at 356.1 N for 60 s. These discs were mounted to specially designed sample holders which fit into a Sotax dissolution tester. The dissolution media (37° C.) were stirred at 200 rpm. Samples were automatically withdrawn every second minute from the dissolution vessel and assayed via UV spectrophotometry. The intrinsic dissolution rate expressed in μg/cm²/min was calculated using the slope of the concentration versus time plot and from the linear portion of the slope of the dissolution curve, volume of dissolution medium (35 ml) and area (diameter: 2 mm) of the exposed disk.

| pH of | dissolution rate |
|---|---|
| aqueous medium | [μg/cm²/min] |
| 1.1 | 8220 |
| 2.3 | 4520 |
| 3.2 | 4000 |
| 4.1 | 3110 |
| 5.1 | 4015 |
| 6.0 | 4140 |
| 7.4 | 240 |

From the above results, it can be concluded that the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide has a pH dependent solubility profile in aqueous media with excellent solubility in acidic media and a reduced solubility in neutral and basic media due to the lower solubility of the free base. Furthermore, the compound exhibits up to pH 6.0 very fast dissolution rates, and even acceptable dissolution rates at pH 7.4.

Solid state properties of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

In the solid state, the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide appears as a white microcrystalline powder.

Manufacturing process of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide.

The anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide may be manufactured by drying a preparation of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide at a temperature above 130° C. and maintaining it under dry atmosphere.

Solid state properties (crystallinity and polymorphism) of the anhydrous crystalline form of the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide

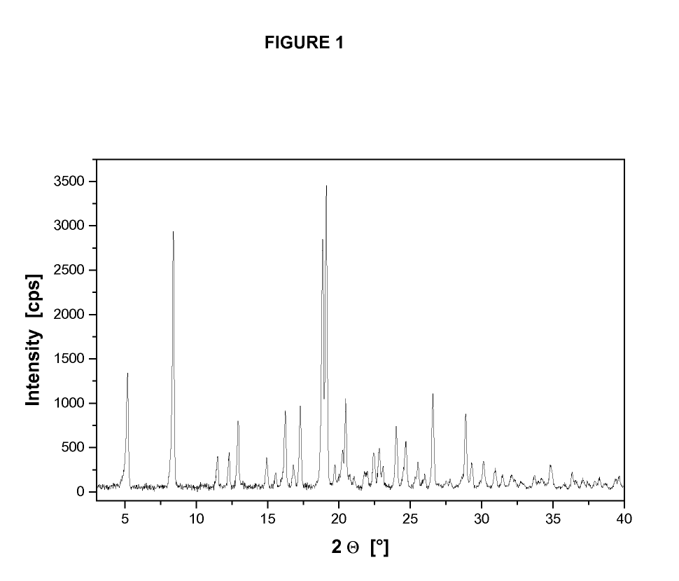

The compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide is highly crystalline. The X-ray powder diffraction diagram is shown in FIG. 1.

The X-ray powder reflection and intensities (standardised) are shown in the following table.

| 2 Θ | d-value | Intensity I/I₀ |
|---|---|---|
| [°] | [Å] | [%] |
| 5.17 | 17.07 | 39 |
| 8.39 | 10.54 | 85 |
| 11.48 | 7.70 | 10 |
| 12.29 | 7.19 | 11 |
| 12.93 | 6.84 | 23 |
| 14.93 | 5.93 | 11 |
| 15.56 | 5.69 | 6 |
| 16.23 | 5.46 | 26 |
| 16.81 | 5.27 | 8 |
| 17.28 | 5.13 | 28 |
| 18.85 | 4.70 | 85 |
| 19.11 | 4.64 | 100 |
| 19.72 | 4.50 | 8 |
| 20.26 | 4.38 | 13 |
| 20.46 | 4.34 | 30 |
| 20.75 | 4.28 | 4 |
| 21.06 | 4.22 | 4 |
| 21.79 | 4.08 | 5 |
| 21.97 | 4.04 | 6 |
| 22.43 | 3.96 | 12 |
| 22.82 | 3.89 | 13 |
| 23.09 | 3.85 | 7 |
| 24.02 | 3.70 | 20 |
| 24.69 | 3.60 | 15 |
| 25.54 | 3.49 | 9 |
| 26.01 | 3.42 | 5 |
| 26.59 | 3.35 | 32 |
| 27.52 | 3.24 | 3 |
| 27.78 | 3.21 | 3 |
| 28.89 | 3.09 | 25 |
| 29.31 | 3.05 | 9 |
| 30.13 | 2.96 | 9 |
| 30.95 | 2.89 | 6 |

In the above table, the value “2Θ [°]” denotes the angle of diffraction in degrees and the value “dₕₖₗ [Å]” denotes the specified distances in Å between the lattice planes.

According to the findings shown in the above table, the present invention further relates to the crystalline anhydrous form of (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide, characterised in that in the x-ray powder diagram has, inter alia, the characteristic values d=3.35 Å, 4.34 Å, 4.64 Å, 4.70 Å, 10.54 Å and 17.07 Å (most prominent peaks in the diagram).

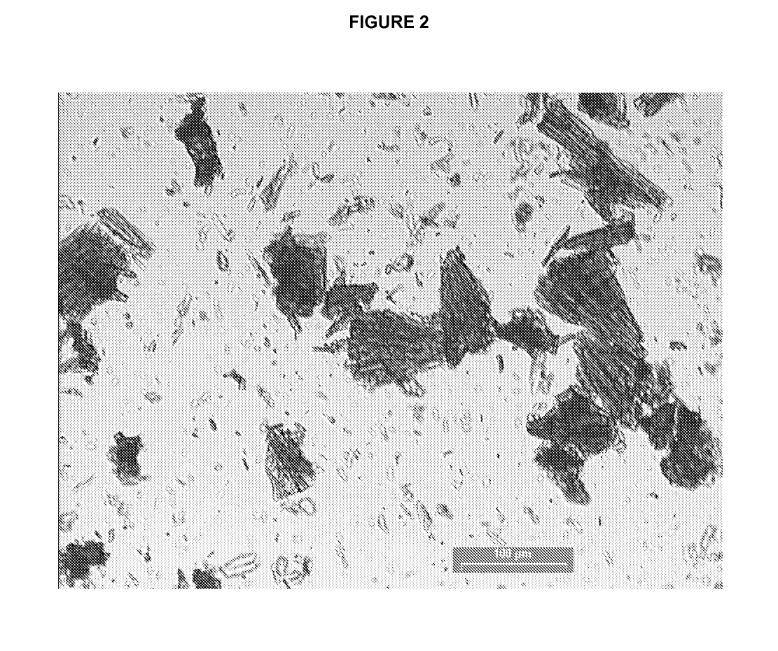

The material crystallizes in rod-like crystals and tends to agglomerate in larger aggregates, as shown in FIG. 2.

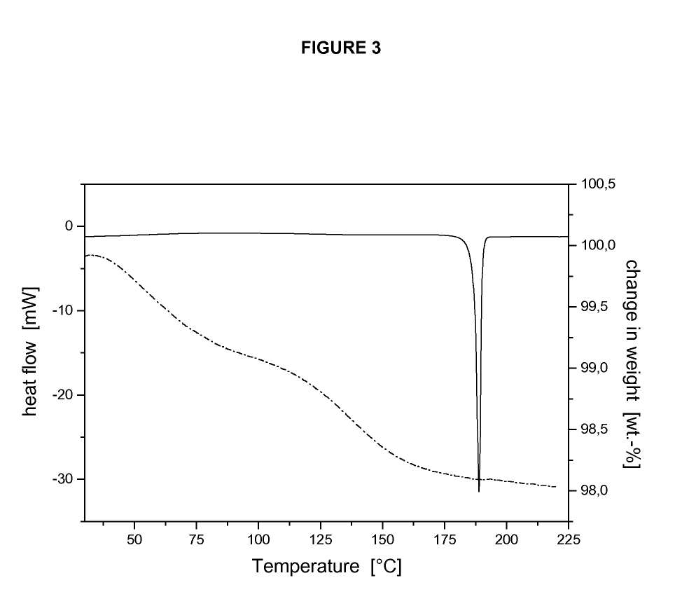

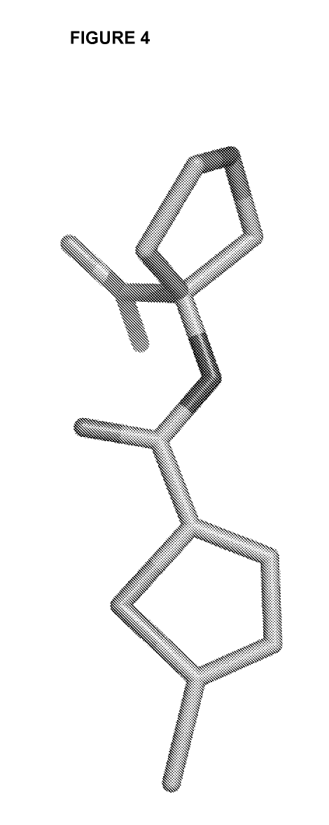

The thermoanalysis of the crystalline anhydrous form of (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide according to the invention, as shown in FIG. 3 (DSC/TG diagram), shows a T_{fus}=185±3° C. (DSC: 10 K·min⁻¹ heating rate), in the form of a strong endothermic peak. A closer look to the TG-trace (confirmed by TG-IR coupling experiments) shows a weight loss of approx. 1.0-2.0% up to approximately 180° C. This weight loss may indicate the presence of absorbed water on the surface of the microcrystalline material. Thermal decomposition starts above 240° C. indicating a congruent melting process at 185° C.

Thus, the present invention further relates to the crystalline anhydrous form of (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide, characterised by a melting point of T_{m.p.}=185±3° C. (determined by DSC; evaluation using peak-maximum; heating rate: 10° C./min).

From the above data, it can be concluded that the compound (S)-3-[(5-chloro-thiophen-2-yl)-carbonylamino]-N-(3-methyl-2,3,4,5-tetrahydro-1H-benzo[d]azepin-7-yl)-tetrahydrofuran-3-carboxylic acid amide is characterized by its high solubility in acidic media and its high crystallinity. The crystalline polymorphic form is characterized as an anhydrous form, present as single stable polymorphic form.

### Example 3

(3S)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide and
(3S)-3-[(5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amide

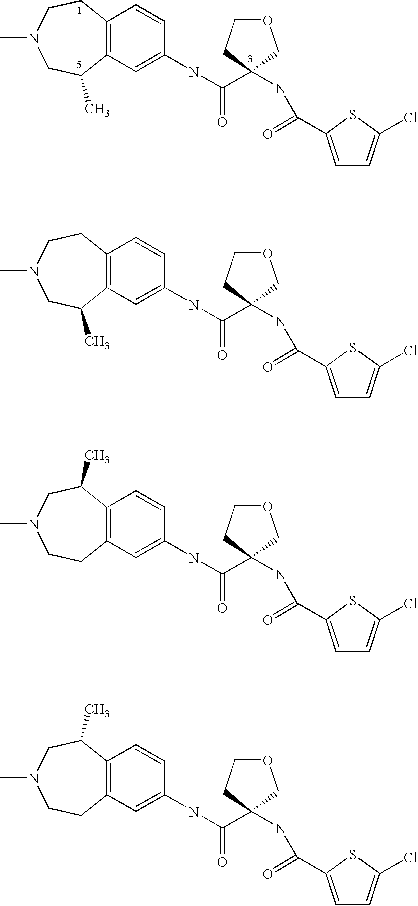

(a) 1-methyl-2-oxo-2,3,4,5-tetrahydro-1H-benzo[d][azepine

8.0 g (37 mmol) 2-chloro-N-(2-phenylethyl)-propanamide and 15 g (112 mmol) aluminium trichloride were carefully mixed at 90° C. and heated to 150° C. for 6 h. The mixture was diluted with water and methanol and extracted with EtOAc. The combined organic layers were dried with Na₂SO₄, concentrated and purified by chromatography (method A) to give the title compound,
(b) 1-methyl-2,3,4,5-tetrahydro-1H-benzo[d][azepine

2.7 g (15 mmol) of 1-methyl-2-oxo-2,3,4,5-tetrahydro-1H-benzo[d][azepine was added to 46 ml 1M BH3-THF complex solution and stirred at room temperature under a nitrogen atmosphere overnight. 50 ml of methanol were carefully added, followed by 30 ml of 2M HCl. The mixture was extracted with EtOAc, the combined organic layers were dried with Na₂SO₄, concentrated and purified by chromatography (method A) to give the title compound as formic acid salt.
(c) 1,3-dimethyl-7-nitro-2,3,4,5-tetrahydro-1H-benzo[d][azepine and 1,3-dimethyl-8-nitro-2,3,4,5-tetrahydro-1H-benzo[d][azepine

Following the procedure for example 1b 1-methyl-2,3,4,5-tetrahydro-1H-benzo[d][azepine was methylated to give 1,3-dimethyl-2,3,4,5-tetrahydro-1H-benzo[d][azepine.

1.79 g (10 mmol) of 1,3-dimethyl-2,3,4,5-tetrahydro-1H-benzo[d][azepine was mixed with 3.7 ml conc. H2SO₄ and 0.71 ml 65% HNO₃ at −5° C. and stirred for 1 h by −5° C. to 0° C. The mixture was poured into 100 ml of ice cold water and 10 M NaOH was added. The mixture was extracted with EtOAc, the combined organic layers were dried with Na₂SO₄, concentrated and purified by chromatography on silica gel (eluant: dichloromethane:95% ethanol/5% ammonia 99:1 to 95:5) to give a mixture of the title compounds.
(d) 3,5-dimethyl-7-amino-2,3,4,5-tetrahydro-1H-benzo[d][azepine and 3,5-dimethyl-8-amino-2,3,4,5-tetrahydro-1H-benzo[d][azepine

1.4 g (6.3 mmol) of a mixture of 1,3-dimethyl-7-nitro-2,3,4,5-tetrahydro-1H-benzo[d][azepine and 1,3-dimethyl-8-nitro-2,3,4,5-tetrahydro-1H-benzo[d][azepine, 20 ml methanol and 0.20 g 10% palladium on charcoal was stirred for 5.5 h under an atmosphere of hydrogen (50 psi). It was filtered, concentrated and the mixture was purified by chromatography on silica gel (eluant: dichloromethane:95% ethanol/5% ammonia 99:1 to 80:20) to give 0.45 g of regioisomer B: rac-7-Amino-1,3-dimethyl-2,3,4,5-tetrahydro-1H-benzo[d]azepine

R_{f} value: 0.75 (silica gel; dichloromethane/ethanol/ammonia=80:20:2)

### C₁₂H₁₈N₂ (190.28)

Mass spectrum: (M+H)⁺=191
and 0.55 g of regioisomer A: rac-7-Amino-3,5-dimethyl-2,3,4,5-tetrahydro-1H-benzo[d]azepine
R_{f} value: 0.70 (silica gel; dichloromethane/ethanol/ammonia=80:20:2)

### C₁₂H₁₈N₂ (190.28)

Mass spectrum: (M+H)⁺=191.
e) Regioisomer A and S-Configurated Carboxylic Acid 7 were Coupled According To the Procedure Described in Example 2d to Give a Mixture of the 3S-Diastereoisomers
   R_{f} value: 0.75 (silica gel; dichloromethane/ethanol/ammonia=80:20:2)

### C₂₂H₂₆ClN₃O₃S (447.979)

Mass spectrum: (M+H)⁺=448/450 chloro isotopes.

For separation of the mixture of diastereoisomers into the pure single stereoisomers (35)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and (3S)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide a conventional HPLC system with DAICEL AD-H 250 mm×4.6 mm chiral column has been used, eluting with 0.2% Cyclohexylamine in Hexane (80%)/EtOH (20%) as liquid phase. At a flow rate of 1 ml/min, retention times for the stereoisomers are 10.75 min and 16.5 min.

Each of the diastereoisomers shows the following

Mass spectrum: (M+H)⁺=448/450 chloro isotopes.

Alternatively, the separation of the mixture of diastereoisomers can be achieved by supercritical fluid chromatography with DAICEL AD-H chiral column, eluting with 0.2% Cyclohexylamine in EtOH (45%)/supercritical CO₂ (65%). At a flow rate of 5 ml/min, retention times for the stereoisomers are 3.94 min and 4.08 min respectively.
f) Regioisomer B and S-Configurated Carboxylic Acid 7 Can be Coupled According to the Procedure Described Above to Give a Diastereomeric Mixture of the Regioisomers. (3S)-3-[(5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide and (3S)-3-[(5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amid. The diastereoisomers could be separated by chiral chromatography (DAICEL AS-H, 250×4.6 mm, eluens: Methanol and 45% Diethylamine). At a flow rate of 5 ml/min, retention times for the stereoisomers are 6.5 min and 8.5 min.

Each of the diastereoisomers shows the following

Mass spectrum: (M+H)⁺=448/450 chloro isotopes.

### Example 3-A

### (3R)-3-[(5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and (3R)-3-[(5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide

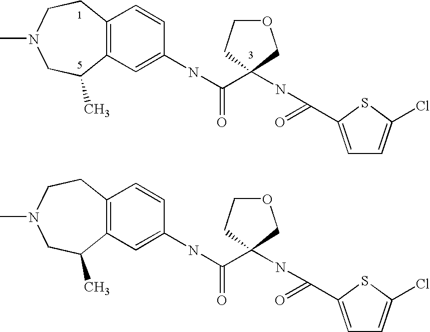

Regioisomer A can be coupled separately with the R-configurated enantiomer of carboxylic acid 7 following the procedure for example 3 to give the 3R-diastereoisomers.

Both diastereoisomers showed the following characteristics

R_{f} value: 0.6 (silica gel; dichloromethane/ethanol/ammonia=80:20:2)

### C₂₂H₂₆ClN₃O₃S (447.979)

Mass spectrum: (M+H)⁺=448/450 chloro isotopes.

### Example 4

### (R)- and (S)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide

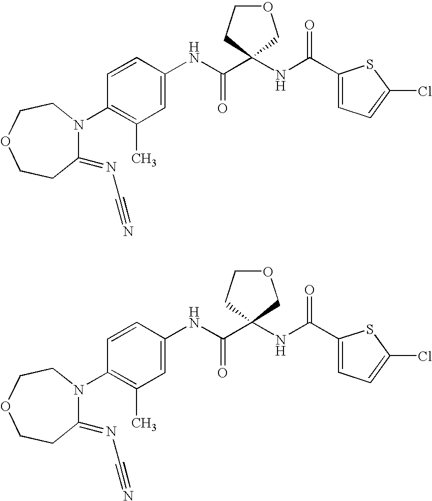

500 mg (1.9 mmol) 2-(5-chloro-thiophene-2-yl)-3,7-dioxa-1-aza-spiro[4.4]non-1-en-4-one are stirred with 0.45 g (1.8 mmol) 3-methyl-4-(5-oxo-[1.4]oxazepan-4-yl)-aniline in 4.5 ml of toluene with 5.0 ml DMF and 500 μl glacial acetic acid at 80° C. for 5 h. Then the reaction mixture was concentrated poured into 100 ml semisat. sodium hydrogen carbonate/100 ml EtOAc. After extraction with EtOAc, the combined organic phases were dried over magnesium sulphate and evaporated down i. vac. The racemic mixture was separated into the enantiomers by using a chiral chromatography column (500×50 mm, DAICEL AD-column, eluent: Ethanol 30 ml/min):

### Enantiomer 1: Rₜ=86 min

Mass spectrum: (M+H)⁺=502/504 (chlorine isotopes)

### Enantiomer 2 Rₜ=136 min.

Mass spectrum: (M+H)⁺=502/504 (chlorine isotopes)

### Example 5

### (R)- and (S)-5-chloro-thiophene-2-carboxylic acid-N-{3-[4-(5-cyanimin-[1.4]oxazepan-4-yl)-phenylcarbamoyl]-tetrahydrofuran-3-yl}-amide

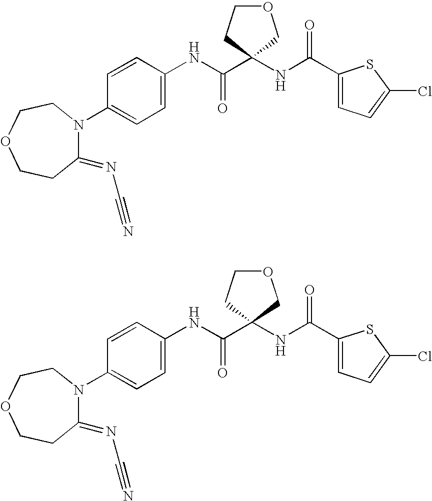

The (S)-Enantiomer was obtained by following the procedure for example 4 and using (S)-2-(5-chloro-thiophene-2-yl)-3,7-dioxa-1-aza-spiro[4.4]non-1-en-4-one 8 and 3-methyl-4-(5-oxo-[1.4]oxazepan-4-yl)aniline.

### C₂₂H₂₂ClN₅O₄S (487.96)

Mass spectrum: (M+H)⁺=488/490 (chlorine isotopes)

The (R)-Enantiomer was obtained by following the same procedure using (R)-2-(5-chloro-thiophene-2-yl)-3,7-dioxa-1-aza-spiro[4.4]non-1-en-4-one and 3-methyl-4-(5-oxo-[1.4]oxazepan-4-yl)aniline.

### C₂₂H₂₂ClN₅O₄S (487.96)

Mass spectrum: (M+H)⁺=488/490 (chlorine isotopes)

### Example 6

### (R)- and (S)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide

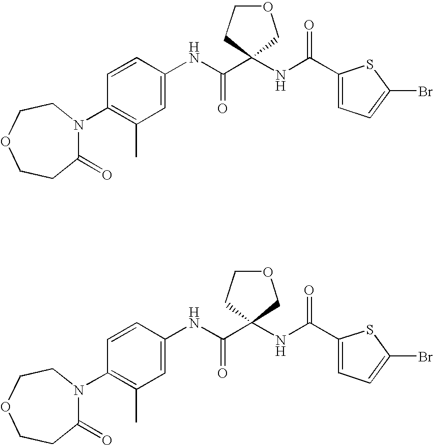

The racemic mixture was prepared according to the following scheme as described in WO2005111029:

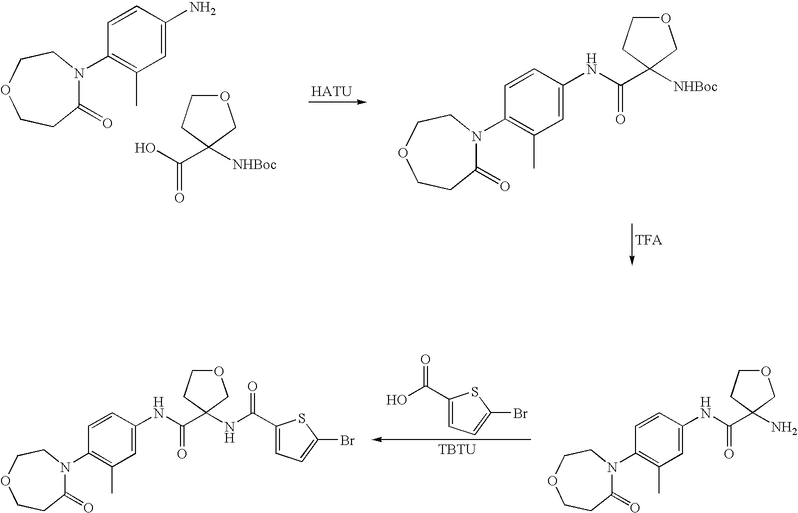

For separation of the racemic mixture into its respective enantiomers, a conventional analytical HPLC system with DAICEL AD-H 250 mm×4.6 mm chiral column has been used, eluting with 0.2% Cyclohexylamine in Hexane (80%)/IPA (20%) as liquid phase. At a flow rate of 1 ml/min, retention times for the enantiomers are 23.70 min and 28.40 min.

### Enantiomer 1: Rₜ=23.70 min

Mass spectrum: (M+H)⁺=520/522 (bromine isotopes)

### Enantiomer 2: Rₜ=28.40 min.

Mass spectrum: (M+H)⁺=520/522 (bromine isotopes)

### Example 7

### (R)- and (S)-5-bromo-thiophene-2-carboxylic acid-N-{3-[4-(5-cyanimin-[1.4]oxazepan-4-yl)-phenylcarbamoyl]-tetrahydrofuran-3-yl}-amide

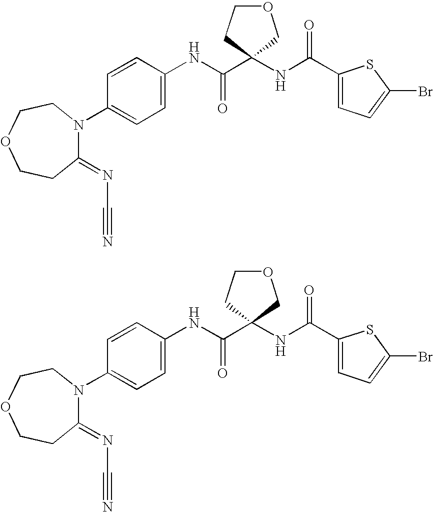

The racemic mixture was prepared from rac-3-[(5-chloro-thiophen-2-yl)-carbonylamino]-tetrahydro-furan-3-carboxylic acid rac-7 and 4-(5cyanimino-[1,4]oxazepan-4-yl)anilin (prepared in analogy to procedures described in WO2005/111029) in analogy to procedure 4.

### C₂₂H₂₂BrN₅O₄S (532.411)

Mass spectrum: (M+H)⁺=532/534 (bromine isotopes)

For separation of the racemic compound into its respective enantiomers, a conventional analytical HPLC system with DAICEL AD-H 250 mm×4.6 mm chiral column has been used, eluting with 0.2% Acetic Acid in Hexane (80%)/EtOH (20%) as liquid phase. At a flow rate of 1 ml/min, retention times for the enantiomers are 9 min and 12.2 min.

Alternatively, the separation of this racemate can be achieved on HPLC with DAICEL OJ-H chiral column, eluting with 0.2% Acetic Acid in Hexane (80%)/EtOH (20%) as liquid phase. At a flow rate of 1 ml/min, retention times for the enantiomers are 6.10 min and 8.10 min respectively.

### Example 8

### (R)- and (S)-5-bromo-thiophene-2-carboxylic acid-N-{3-[3-methyl-4-(3-oxo-morpholin-4-yl)-phenylcarbamoyl]-tetrahydro-thiophen-3-yl}-amide

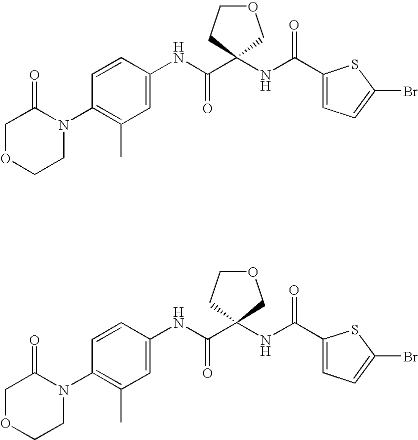

The racemic mixture was prepared as described in WO2005/111029. For separation of the racemic mixture into its respective enantiomers, a conventional HPLC system with a DAICEL OD 250 mm×20 mm chiral column has been used, eluting first with hexane and after the first peak has been eluated with hexane/ethanol 55/45 as liquid phases.

### C₂₁H₂₂BrN₃O₅S (508.39)

Mass spectrum: (M+H)⁺=508/510 (bromine isotopes) for each enantiomer.

### Example 9

### (R)- and (S)-5-ethinyl-thiophene-2-carboxylic acid-N-{3-[4-(3-oxo-morpholin-4-yl)-phenylcarbamoyl]-tetrahydro-thiophen-3-yl}-amide

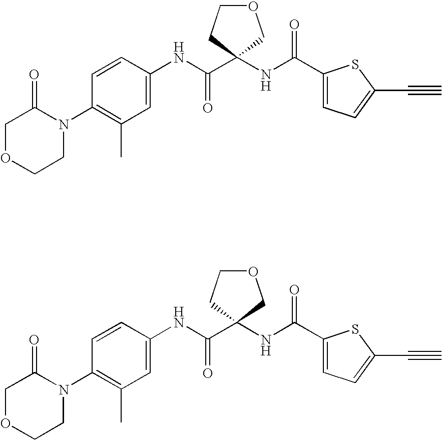

The racemic mixture was prepared as described in WO2006/034822. For separation of the racemic compound into its respective enantiomers, a conventional analytical HPLC system with DAICEL AD-H 250 mm×4.6 mm chiral column has been used, eluting with hexane/ethanol 1/1 as liquid phase. Retention times for the enantiomers were 13.9 min and 22.2 min. For the preparative separation a DAICEL AD-H 500 mm×50 mm chiral column has been used, eluting with ethanol as liquid phase.

The following racemic compounds can be prepared in enantiomerically pure form analogously to the methods described in the Examples above or by synthetic pathways known from the literature:

### A (S)- and (R)-5-bromo-thiophene-2-carboxylic acid-N-{3-[3-methyl-4-(pyrrolidin-1-yl-carbonyl)-phenylcarbamoyl]-tetrahydrofuran-3-yl}-amide

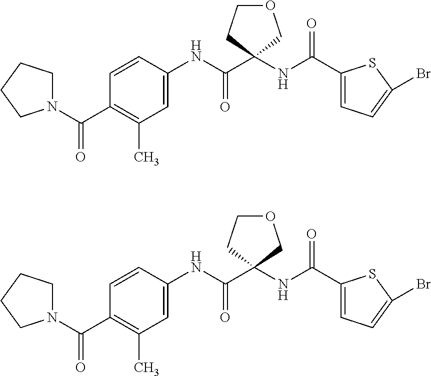

### B (S)- and (R)-5-bromo-thiophene-2-carboxylic acid-N-{3-[4-(pyrrolidin-1-yl-carbonyl)-phenylcarbamoyl]-tetrahydrofuran-3-yl}-amide

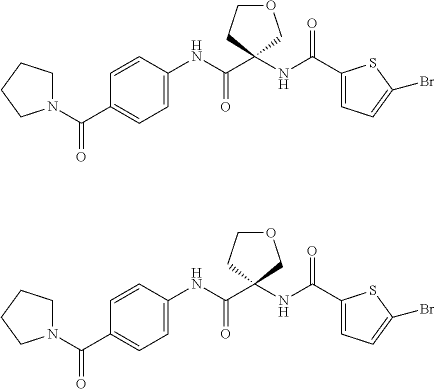

### C (S)- and (R)-5-ethinyl-thiophene-2-carboxylic acid-N-{3-[3-chloro-4-(3-oxo-morpholin-4-yl)-phenylcarbamoyl]-tetrahydro-thiophen-3-yl}-amide

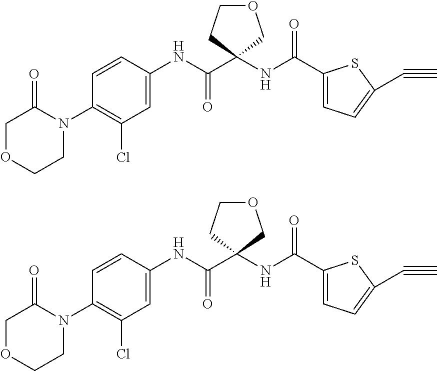

### D (S)- and (R)-5-bromo-thiophene-2-carboxylic acid-N-{3-[3-chloro-4-(2-oxo-azepan-1-yl)-phenylcarbamoyl]-tetrahydro-thiophen-3-yl}-amide

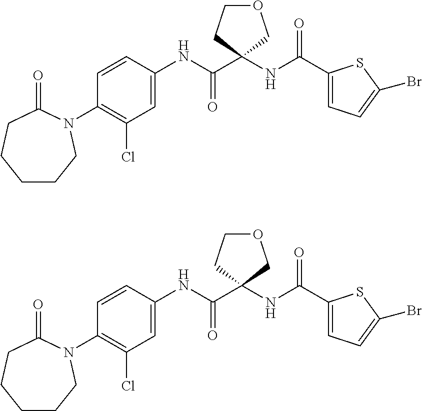

### E (S)- and (R)-5-bromo-thiophene-2-carboxylic acid-N-{3-[4-(2-oxo-azepan-1-yl)-phenylcarbamoyl]-tetrahydro-thiophen-3-yl}-amide

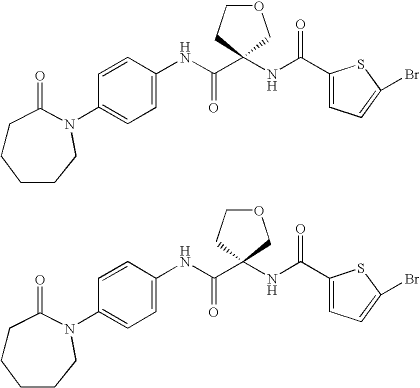

### F (S)- and (R)-5-bromo-thiophene-2-carboxylic acid-N-{3-[3-methyl-4-(5-cyanimin-[1.4]oxazepan-4-yl)-phenylcarbamoyl]-tetrahydrofuran-3-yl}-amide

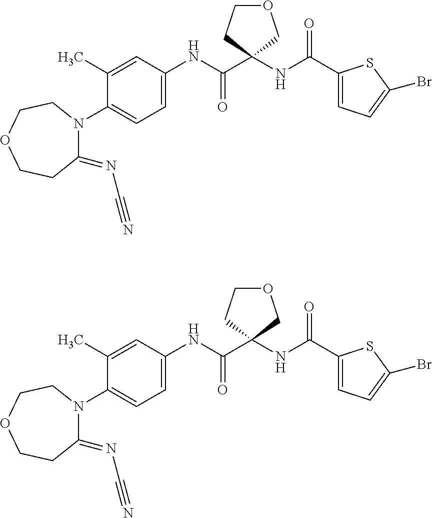

### G (S)- and (R)-5-bromo-thiophene-2-carboxylic acid-N-{3-[3-chloro-4-(5-cyanimin-[1.4]oxazepan-4-yl)-phenylcarbamoyl]-tetrahydrofuran-3-yl}-amide

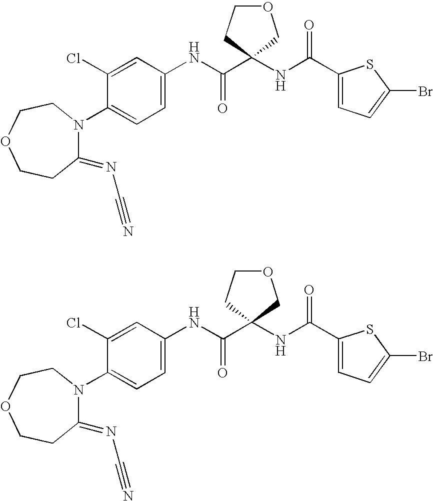

### H (S)- and (R)-5-chloro-thiophene-2-carboxylic acid-N-{3-[3-methyl-4-(3-cyanimino-morpholin-4-yl)-phenylcarbamoyl]-tetrahydrofuran-3-yl}-amide

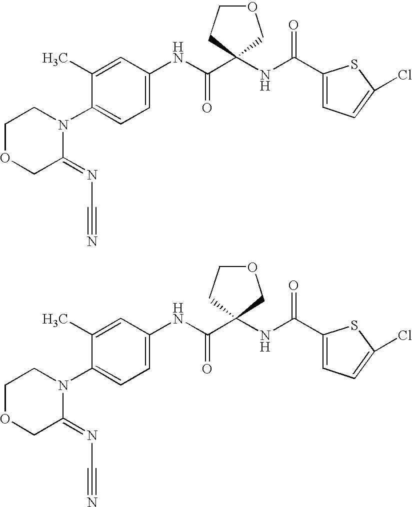

### I (S)- and (R)-5-chloro-thiophene-2-carboxylic acid-N-{3-[3-chloro-4-(3-cyan imino-morpholin-4-yl)-phenylcarbamoyl]-tetrahydrofuran-3-yl}-amide

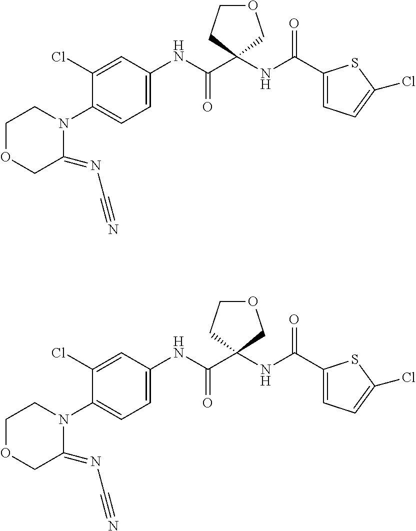

### J (S)-3-[(5-chloro-thiophen-2-yl)-carbonylamino]-N-((2S)-2,3-dimethyl-2,3,4,5-tetrahydro-1H-benzo[d]azepin-7-yl)-tetrahydrofuran-3-carboxylic acid amide and (S)-3-[(5-chloro-thiophen-2-yl)-carbonylamino]-N-((2R)-2,3-dimethyl-2,3,4,5-tetrahydro-1H-benzo[d]azepin-7-yl)-tetrahydrofuran-3-carboxylic acid amide

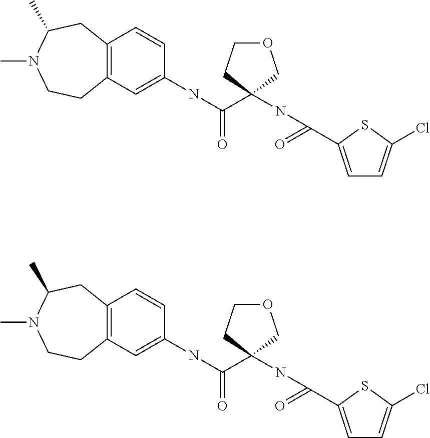

### K (S)-3-[(5-chloro-thiophen-2-yl)-carbonylamino]-N-((4S)-3,4-dimethyl-2,3,4,5-tetrahydro-1H-benzo[d]azepin-7-yl)-tetrahydrofuran-3-carboxylic acid amide and (S)-3-[(5-chloro-thiophen-2-yl)-carbonylamino]-N-((4R)-3,4-dimethyl-2,3,4,5-tetrahydro-1H-benzo[d]azepin-7-yl)-tetrahydrofuran-3-carboxylic acid amide

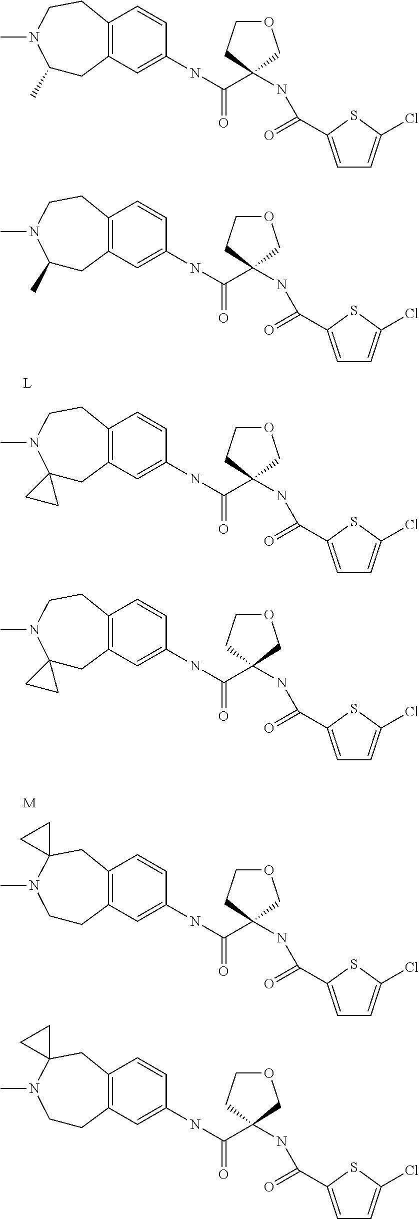

### N (S)- and (R)-3-[(5-chloro-thiophen-2-yl)-carbonylamino]-N-(2,2,3-trimethyl-2,3,4,5-tetrahydro-1H-benzo[d]azepin-7-yl)-tetrahydrofuran-3-carboxylic acid amide

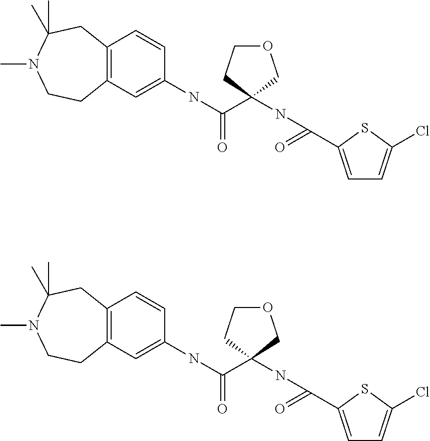

### O (S)- and (R)-3-[(5-chloro-thiophen-2-yl)-carbonylamino]-N-(3,4,4-trimethyl-2,3,4,5-tetrahydro-1H-benzo[d]azepin-7-yl)-tetrahydrofuran-3-carboxylic acid amide

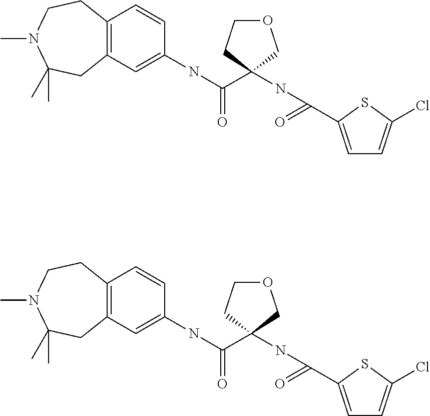

### P (S)- and (R)-3-[(5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide

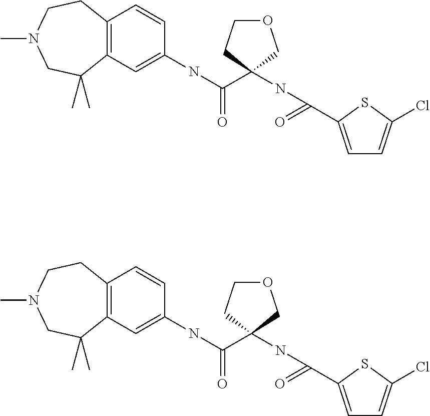

(S)-3-[(5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide was prepared in analogy to the procedures described above.
R_{f} value: 0.38 (RP-8; methanol/5% NaCl-solution=6:4)

### C₂₃H₂₈ClN₃O₃S (462.01)

Mass spectrum: (M+H)⁺=462/464 (chlorine isotopes)

### Q (S)- and (R)-3-[(5-chloro-thiophen-2-yl)-carbonylamino]-N-(3-methyl-9-fluor-2,3,4,5-tetrahydro-1H-benzo[d]azepin-7-yl)-tetrahydrofuran-3-carboxylic acid amide

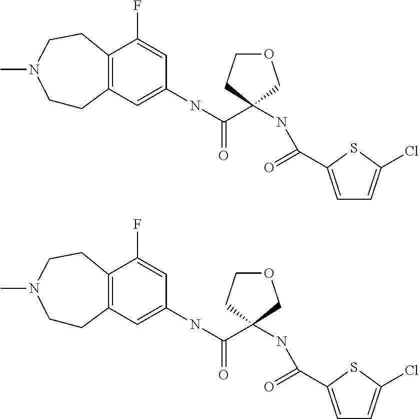

### R (S)- and (R)-3-[(5-chloro-thiophen-2-yl)-carbonylamino]-N-(3-methyl-8-fluor-2,3,4,5-tetrahydro-1H-benzo[d]azepin-7-yl)-tetrahydrofuran-3-carboxylic acid amide

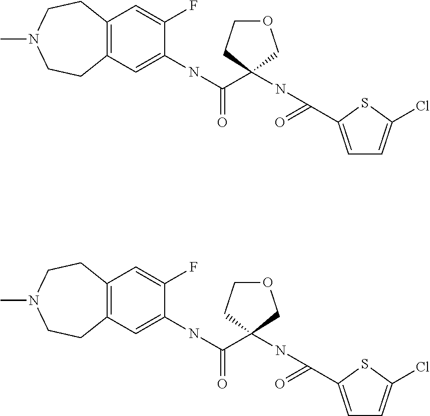

### S (S)-3-[(5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amide and (S)-3-[(5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide

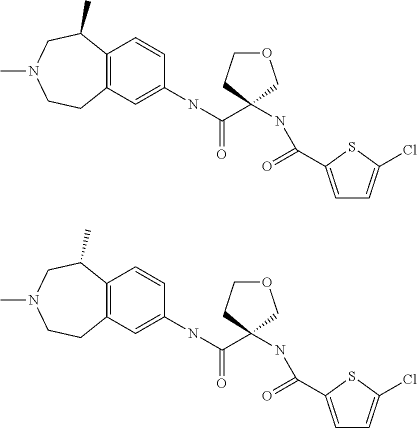

The Examples that follow describe the preparation of pharmaceutical formulations which contain as active substance any desired compound of general formula I.

### Example A

### Dry Ampoule Containing 75 Mg of Active Substance Per 10 Ml

### Composition:

| | |
|---|---|
| Active substance | 75.0 mg |
| Mannitol | 50.0 mg |
| water for injections | ad 10.0 ml |

### Preparation:

Active substance and mannitol are dissolved in water. After packaging the solution is freeze-dried. To produce the solution ready for use for injections, the product is dissolved in water.

### Example B

### Dry Ampoule Containing 35 Mg of Active Substance Per 2 ml

### Composition:

| | | |
|---|---|---|
| Active substance | 35.0 | mg |
| Mannitol | 100.0 | mg |
| water for injections | ad 2.0 | ml |

### Preparation:

Active substance and mannitol are dissolved in water. After packaging, the solution is freeze-dried.

To produce the solution ready for use for injections, the product is dissolved in water.

### Example C

### Tablet Containing 50 Mg of Active Substance

### Composition:

| | | |
|---|---|---|
| (1) Active substance | 50.0 | mg |
| (2) Lactose | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### Example D

### Tablet Containing 350 Mg of Active Substance

### Composition:

| | | |
|---|---|---|
| (1) Active substance | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### Example E

### Capsules Containing 50 Mg of Active Substance

### Composition:

| | | |
|---|---|---|
| (1) Active substance | 50.0 | mg |
| (2) Dried maize starch | 58.0 | mg |
| (3) Powdered lactose | 50.0 | mg |
| (4) Magnesium stearate | 2.0 | mg |
| | 160.0 | mg |

### Preparation:

(1) is triturated with (3). This trituration is added to the mixture of (2) and (4) with vigorous mixing.

This powder mixture is packed into size 3 hard gelatine capsules in a capsule filling machine.

### Example F

### Capsules Containing 350 Mg of Active Substance

### Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

### Preparation:

(1) is triturated with (3). This trituration is added to the mixture of (2) and (4) with vigorous mixing.

This powder mixture is packed into size 0 hard gelatine capsules in a capsule filling machine.

### Example G

### Suppositories Containing 100 Mg of Active Substance

1 suppository contains:

| | | |
|---|---|---|
| Active substance | 100.0 | mg |
| Polyethyleneglycol (M.W. 1500) | 600.0 | mg |
| Polyethyleneglycol (M.W. 6000) | 460.0 | mg |
| Polyethylenesorbitan monostearate | 840.0 | mg |
| | 2,000.0 | mg |

### Preparation:

The polyethyleneglycol is melted together with polyethylenesorbitan monostearate. At 40° C. the ground active substance is homogeneously dispersed in the melt. It is cooled to 38° C. and poured into slightly chilled suppository moulds.

1. Substituted 3-amino-tetrahydrofuran-3-carboxylic acid amides of general formula (I) 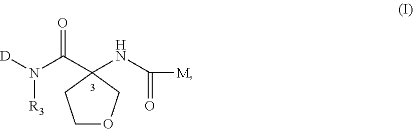
in high optical purity at the carbon atom in position 3 of the tetrahydrofuran ring, wherein
D denotes D¹ a substituted bicyclic ring system of formula (II), 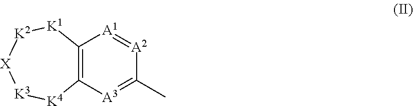
wherein
K¹ and K⁴
each independently of one another denote a —CH₂, —CHR^{7a}, —CR^{7b}R^{7c} or a —C(O) group, and
R^{7a}/R^{7b}/R^{7c}
each independently of one another denote a fluorine atom, a hydroxy, C₁₋₅-alkyloxy, amino, C₁₋₅-alkylamino, di-(C₁₋₅-alkyl)-amino, C₃₋₅-cycloalkyleneimino or C₁₋₅-alkylcarbonyl amino group,
a C₁₋₅-alkyl group which may be substituted by 1-3 fluorine atoms, or
two groups R^{7b}/R^{7c} together with the cyclic carbon atom may form a 3, 4, 5-, 6- or 7-membered saturated carbocyclic group
wherein the methylene groups thereof may be substituted by 1-2 C₁₋₃-alkyl or CF₃— groups, and/or
the methylene groups thereof, if they are not bound to a heteroatom, may be substituted by 1-2 fluorine atoms, and
K² and K³
each independently of one another denote a —CH₂, —CHR^{8a}, —CR^{8b}R^{8c} or a —C(O)— group, and
R^{8a}/R^{8b}/R^{8c}
each independently of one another denote a C₁₋₅-alkyl group which may be substituted by 1-3 fluorine atoms,
or two groups R^{8b}/R^{8c} together with the cyclic carbon atom may form a 3, 4, 5-, 6- or 7-membered saturated carbocyclic group, and
in all there should be no more than four groups selected from among R^{7a}, R^{7b}, R^{7c}, R^{8a}, R^{8b} and R^{8c} in formula (II), and
X denotes a NR¹ group, wherein
R¹ denotes a hydrogen atom or a hydroxy, C₁₋₃-alkyloxy, amino, C₁₋₃-alkylamino, di-(C₁₋₃-alkyl)-amino, a C₁₋₅-alkyl, C₂₋₅-alkenyl-CH₂, C₂₋₅-alkynyl-CH₂ or C₃₋₆-cycloalkyl group, wherein the methylene and methyl groups present in the above-mentioned groups may additionally be substituted by a C₁₋₃-alkyl, carboxy, C₁₋₅-alkoxycarbonyl group or by a hydroxy, C₁₋₅-alkyloxy, amino, C₁₋₅-alkylamino, C₁₋₅-dialkylamino or C₄₋₇-cycloalkyleneimino group,
provided that O—C—O or O—C—N or N—C—N-bonds are excluded and/or
one to three hydrogen atoms may be replaced by fluorine atoms, provided that the methylene or methyl groups are not directly bound to a nitrogen atom,
and wherein
A¹ denotes either N or CR¹⁰,
A² denotes either N or CR¹¹,
A³ denotes either N or CR¹²,
wherein R¹⁰, R¹¹ and R¹² each independently of one another represent
a hydrogen, fluorine, chlorine, bromine or iodine atom, or a C₁₋₅-alkyl, CF₃, C₂₋₅-alkenyl, C₂₋₅-alkynyl, a cyano, carboxy, C₁₋₅-alkyloxycarbonyl, hydroxy, C₁₋₃-alkyloxy, CF₃O, CHF₂O, CH₂FO, or
D denotes D² a group of general formula 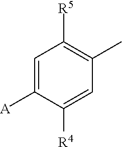
wherein A denotes A⁴, a group 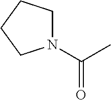
or wherein A denotes A⁵, a group of general formula 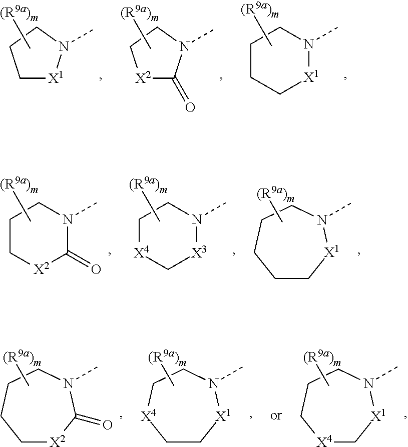
wherein
m is the number 1 or 2,
X¹ denotes a carbonyl, thiocarbonyl, C═NR^{9c}, C═N—OR^{9c}, C═N—NO₂, C═N—CN or sulphonyl group,
X² denotes an oxygen atom or a —NR^{9b} group,
X³ denotes a carbonyl, thiocarbonyl, C═NR^{9c}, C═N—OR^{9c}, C═N—NO₂, C═N—CN or sulphonyl group,
X⁴ denotes an oxygen or sulphur atom or a —NR^{9c} group,
R^{9a} in each case independently of one another denotes a hydrogen or halogen atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
in the previously mentioned substituted 5- to 7-membered groups A⁵ the heteroatoms F, Cl, Br, I, O or N optionally introduced with R^{9a} as substituent are not separated by precisely one carbon atom from a heteroatom selected from among N, O, S,
R^{9b} each independently of one another denote a hydrogen atom or a C₁₋₅-alkyl group,
R^{9c} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R⁴ denotes a hydrogen or halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R⁵ denotes a hydrogen or halogen atom or a C₁₋₃-alkyl group,
R³ denotes a hydrogen atom or a C₁₋₃-alkyl group, and
M denotes a thiophene ring according to formula (III), 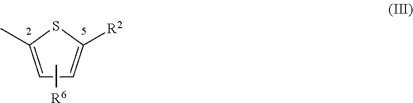
which is bound to the carbonyl group in formula (I) via the 2-position and which is substituted in the 5-position by R² and optionally additionally by R⁶, wherein
R² denotes
R^{2a} a hydrogen, fluorine or iodine atom, or
R^{2b} a methoxy, C₁₋₂-alkyl, formyl, NH₂CO, or
R^{2c} a chlorine, bromine or ethynyl group,
R⁶ denotes a hydrogen, fluorine, chlorine, bromine or iodine atom or a C₁₋₂-alkyl or amino group,
wherein, unless otherwise stated, by the term “halogen atom” mentioned hereinbefore in the definitions is meant an atom selected from among fluorine, chlorine, bromine and iodine,
and wherein the alkyl, alkenyl, alkynyl and alkyloxy groups contained in the previously mentioned definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
the tautomers, enantiomers, diastereomers, mixtures and salts thereof.

2. Substituted 3-amino-tetrahydrofuran-3-carboxylic acid amides of general formula (I) in accordance with claim 1 in high optical purity at the carbon in position 3 of the tetrahydrofuran ring, wherein
D denotes a substituted bicyclic ring system of formula (II), 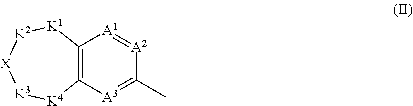
wherein
K¹ and K⁴
each independently of one another denote a —CH₂, —CHR^{7a}, or a —CR^{7b}R^{7c} group, wherein
R^{7a}/R^{7b}/R^{7c}
each independently of one another denote a fluorine atom, a hydroxy, methoxy or C₁₋₂-alkyl group which may be substituted by 1-3 fluorine atoms,
wherein the two groups R^{7b}/R^{7c} cannot both simultaneously be bound to the cyclic carbon atom via a heteroatom, except if —C(R^{7b}R^{7c})— corresponds to a —CF₂ group, or
two groups R^{7b}/R^{7c} may form, together with the cyclic carbon atom, a 3-, 4- or 5-membered saturated carbocyclic group, and
K² and K³
each independently of one another represent a, —CH₂, —CHR^{8a}, or CR^{8b}R^{8c} group, and
R^{8a}/R^{8b}/R^{8c}
each independently of one another denote a C₁₋₂-alkyl group which may be substituted by 1-3 fluorine atoms,
or two groups R^{8b}/R^{8c} may form, together with the cyclic carbon atom, a 3-, 4-, 5-membered carbocyclic group, and
in all in formula (II) there should be no more than four groups selected from among R^{7a}, R^{7b}, R^{7c}, R^{8a}, R^{8b} and R^{8c}, and
X denotes an NR¹ group, wherein
R¹ denotes a hydrogen atom or
a C₁₋₂-alkyl or C₃₋₄-cycloalkyl group,
wherein the methylene and methyl groups present in the above-mentioned groups may additionally be substituted by a methyl group,
and wherein
A¹ denotes CR¹⁰,
A² denotes CR¹¹,
A³ denotes CR¹²,
wherein R¹⁰, R¹¹ and R¹² each independently of one another represent
a hydrogen, fluorine, chlorine, bromine atom or a Methyl, CF₃, a cyano, Methoxy, CF₃O, CHF₂O, CH₂FO— group, or
D denotes D² a group of general formula 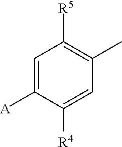
wherein A denotes A⁴, a group 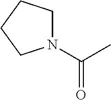
or wherein A denotes A⁵, a group of general formula 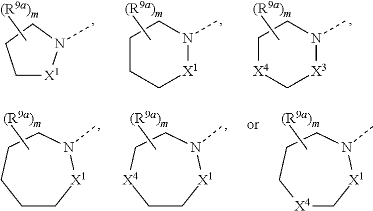
wherein
m is the number 1 or 2,
X¹ denotes a carbonyl or C═N—CN group,
X³ denotes a carbonyl or C═N—CN group,
X⁴ denotes an oxygen atom,
R^{9a} in each case independently of one another denotes a hydrogen atom or a C₁₋₂-alkyl group, while
R⁴ denotes a hydrogen or fluorine, chlorine or bromine atom, a methyl or a methoxy group,
R⁵ denotes a hydrogen, fluorine or chlorine atom or a methyl group,
R³ denotes a hydrogen atom and
M denotes a thiophene ring according to formula (III), 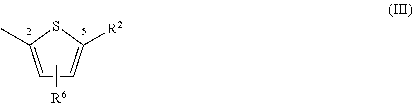
which is bound to the carbonyl group in formula (I) via the 2-position and which is substituted in the 5-position by R² and optionally additionally by R⁶, wherein
R² denotes
R^{2c} a chlorine, bromine atom or an ethynyl group,
R⁶ denotes a hydrogen atom,
the tautomers, diastereomers, mixtures and salts thereof.

3. Substituted 3-amino-tetrahydrofuran-3-carboxylic acid amides of general formula (I) in accordance with claim 1 in high optical purity at the carbon in position 3 of the tetrahydrofuran ring, wherein
D denotes a substituted bicyclic ring system of formula (II), 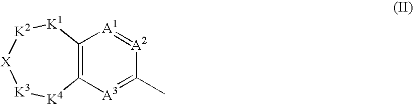
wherein K1, K2, K3, K4, X, A1, A2 and A3 are as defined in claim 1,
the tautomers, diastereomers, mixtures and salts thereof.

4. Substituted 3-amino-tetrahydrofuran-3-carboxylic acid amides of general formula (I) in accordance with claim 1 in high optical purity at the carbon in position 3 of the tetrahydrofuran ring, wherein
D denotes D² a group of general formula 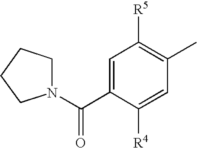
wherein R⁴ and R⁵ are as defined in claim 1,
the tautomers, diastereomers, mixtures and salts thereof.

5. Substituted 3-amino-tetrahydrofuran-3-carboxylic acid amides of general formula (I) in accordance with claim 1 in high optical purity at the carbon in position 3 of the tetrahydrofuran ring, wherein
D denotes D² a group of general formula 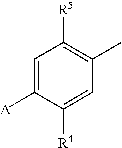
or wherein A denotes A⁵, wherein A5, R⁴ and R5 are as defined in claim 1,
the tautomers, diastereomers, mixtures and salts thereof.

6. Substituted 3-amino-tetrahydrofuran-3-carboxylic acid amides of general formula (I) in accordance with claim 1 in high optical purity at the carbon in position 3 of the tetrahydrofuran ring, wherein the amino-tetrahydrofuran carboxylic acid amide moiety has the R-configuration.

7. Substituted 3-amino-tetrahydrofuran-3-carboxylic acid amides of general formula (I) in accordance with claim 1 in high optical purity at the carbon in position 3 of the tetrahydrofuran ring, wherein the amino-tetrahydrofuran carboxylic acid amide moiety has the S-configuration.

8. A compound in accordance with claim 1, which is selected from the following list of compounds and mixtures and salts thereof:
(S)-3-[5-bromo-thiophen-2-yl)-carbonylamino]-N-(3-methyl-2,3,4,5-tetrahydro-1H-benzo[d]azepin-7-yl)-tetrahydrofuran-3-carboxylic acid amide 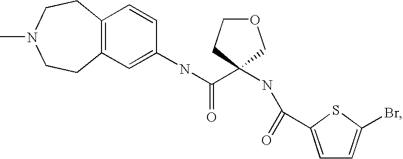
(S)-3-[5-chloro-thiophen-2-yl)-carbonylamino]-N-(3-methyl-2,3,4,5-tetrahydro-1H-benzo[d]azepin-7-yl)-tetrahydrofuran-3-carboxylic acid amide 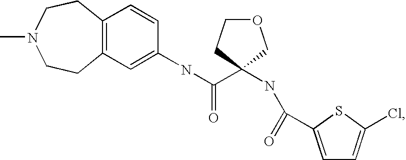
(3S)-3-[5-chloro-thiophen-2-yl)-carbonylamino]-N-((5R)-3,5-dimethyl-2,3,4,5-tetrahydro-1H-benzo[d]azepin-7-yl)-tetrahydrofuran-3-carboxylic acid amide and
(3S)-3-[5-chloro-thiophen-2-yl)-carbonylamino]-N-((5S)-3,5-dimethyl-2,3,4,5-tetrahydro-1H-benzo[d]azepin-7-yl)-tetrahydrofuran-3-carboxylic acid amide 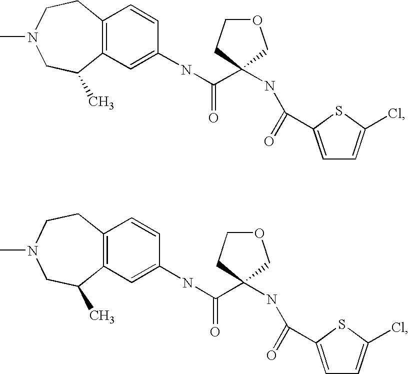
(3S)-3-[5-chloro-thiophen-2-yl)-carbonylamino]-N-((1R)-1,3-dimethyl-2,3,4,5-tetrahydro-1H-benzo[d]azepin-7-yl)-tetrahydrofuran-3-carboxylic acid amide and
(3S)-3-[5-chloro-thiophen-2-yl)-carbonylamino]-N-((1S)-1,3-dimethyl-2,3,4,5-tetrahydro-1H-benzo[d]azepin-7-yl)-tetrahydrofuran-3-carboxylic acid amide 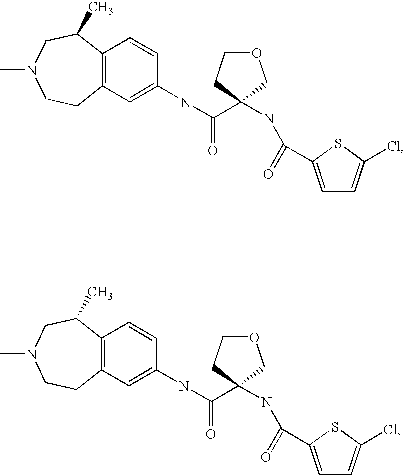
(S)-5-chloro-thiophene-2-carboxylic acid-N-{3-[3-methyl-4-(5-cyanimin-[1.4]oxazepan-4-yl)-phenylcarbamoyl]-tetrahydrofuran-3-yl}-amide 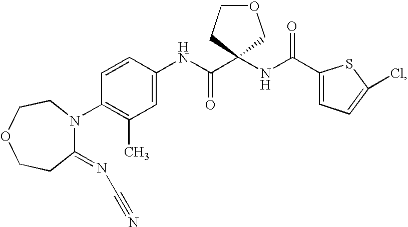
(S)-5-bromo-thiophene-2-carboxylic acid-N-{3-[3-methyl-4-(5-oxo-[1,4]oxazepan-4-yl)-phenylcarbamoyl]-tetrahydrofuran-3-yl}-amide 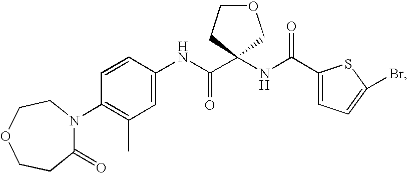
(S)-5-chloro-thiophene-2-carboxylic acid-N-{3-[4-(5-cyanimin-[1,4]oxazepan-4-yl)-phenylcarbamoyl]-tetrahydrofuran-3-yl}-amide 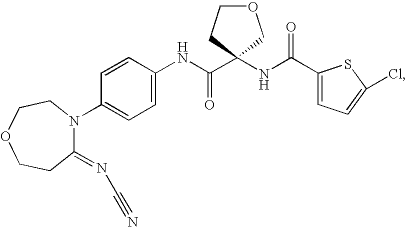
(S)-5-bromo-thiophene-2-carboxylic acid-N-{3-[3-methyl-4-(3-oxo-morpholin-4-yl)-phenylcarbamoyl]-tetrahydro-thiophen-3-yl}-amide 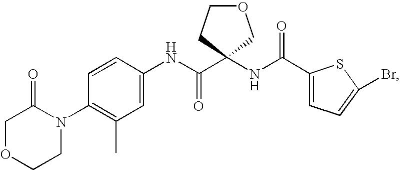
and
(S)-3-[5-chloro-thiophen-2-yl)-carbonylamino]-N-(3,5,5-trimethyl-2,3,4,5-tetrahydro-1H-benzo[d]azepin-7-yl)-tetrahydrofuran-3-carboxylic acid amide

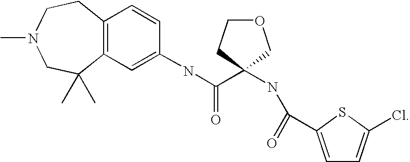

9. A method for the preparation of substituted 3-amino-tetrahydrofuran-3-carboxylic acid amides of general formula (I) in accordance with claim 1 in high optical purity at the carbon in position 3 of the tetrahydrofuran ring, wherein the enantiomers are separated via chiral column chromatography.

10. A method in accordance with claim 9, wherein the column used for the chiral chromatographic separation is selected from the group consisting of the DAICEL columns AD-H, OD-H, AS-H, OJ-H, IA, IB and Kromasil DMB, TBB.

11. A method in accordance with claim 9, wherein the column used for the chiral chromatographic separation is selected from the group consisting of the DAICEL AD-H, OJ-H and IA columns.

12. A method for the preparation of substituted 3-amino-tetrahydrofuran-3-carboxylic acid amides of general formula (Ia) in high optical purity at the carbon in position 3 of the tetrahydrofuran ring 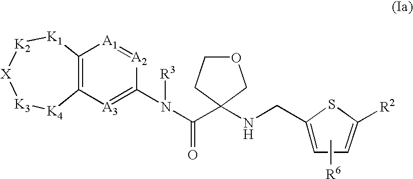
comprising reacting a compound of the general formula (IVa) 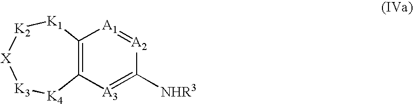
with a compound of the general formula (V) in high optical purity at the carbon in position 3 of the tetrahydrofuran ring 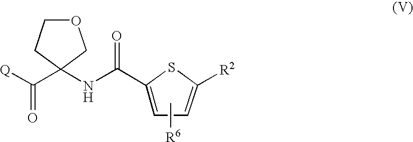
optionally further comprising cleaving protecting groups,
wherein K1, K2, K3, K4, X, A1, A2, A3, R2, R3 and R6 are as defined in claim 1, and
wherein Q is a hydroxy or C₁₋₄-alkyloxy group, a halogen atom or a C₁₋₅-alkyloxycarbonyloxy or acyloxy group.

13. The method according to claim 12, wherein the amino-tetrahydrofuran carboxylic acid amide moiety of the compound of the general formula (V) and of the 3-amino-tetrahydrofuran-3-carboxylic acid amides of general formula (Ia) have the R-configuration.

14. The method according to claim 12, wherein the amino-tetrahydrofuran carboxylic acid amide moiety of the compound of the general formula (V) and of the 3-amino-tetrahydrofuran-3-carboxylic acid amides of general formula (Ia) have the S-configuration.

15. A method for the preparation of substituted 3-amino-tetrahydrofuran-3-carboxylic acid amides of general formula (Ib) in high optical purity at the carbon in position 3 of the tetrahydrofuran ring 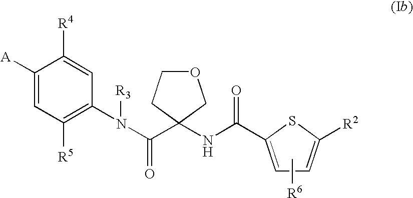
comprising reacting a compound of the general formula (IVb) 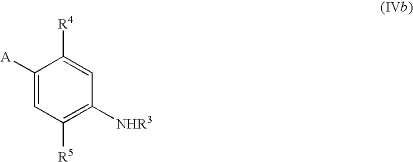
with a compound of the general formula (V) in high optical purity at the carbon in position 3 of the tetrahydrofuran ring 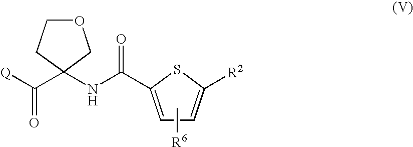
optionally further comprising cleaving protecting groups,
wherein A, R4, R5, R2, R3 and R6 are as defined in claim 1 and
wherein Q is a hydroxy or C₁₋₄-alkyloxy group, a halogen atom or a C₁₋₅-alkyloxycarbonyloxy or acyloxy group.

16. The method according to claim 15, wherein the amino-tetrahydrofuran carboxylic acid amide moiety of the compound of the general formula (V) and of the 3-amino-tetrahydrofuran-3-carboxylic acid amides of general formula (Ib) have the R-configuration.

17. The method according to claim 15, wherein the amino-tetrahydrofuran carboxylic acid amide moiety of the compound of the general formula (V) and of the 3-amino-tetrahydrofuran-3-carboxylic acid amides of general formula (Ib) have the S-configuration.

18. A method for the preparation of substituted 3-amino-tetrahydrofuran-3-carboxylic acid amides of general formula (Ic) in high optical purity at the carbon in position 3 of the tetrahydrofuran ring 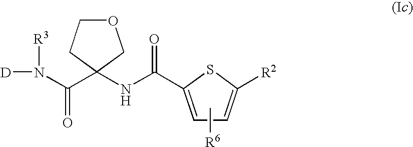
comprising the steps of:
a) reacting a compound of the formula (IV) with a compound of the formula (VI) in high optical purity at the carbon in position 3 of the tetrahydrofuran ring, 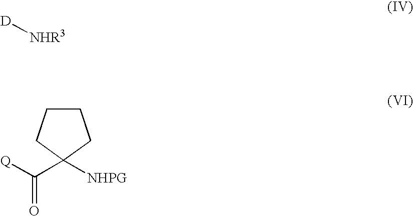
and subsequently cleaving the protecting group PG to obtain a compound of the formula (VII) in high optical purity at the carbon in position 3 of the tetrahydrofuran ring; and 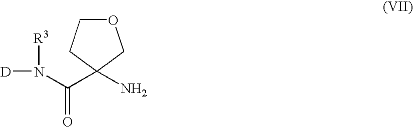
b) reacting said compound (VII) of step a) with a compound of formula (VIII) 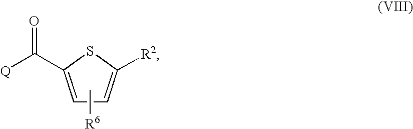
wherein
Q is a hydroxy or C₁₋₄-alkyloxy group, a halogen atom or a C₁₋₅-alkyloxycarbonyloxy or acyloxy group,
PG is a hydrogen atom or a protective group for the amino function, and
D, R3, R2 and R6 are as defined in claim 1.

19. The method according to claim 18, wherein the amino-tetrahydrofuran carboxylic acid amide moiety of the compound of the general formula (VI) and of the 3-amino-tetrahydrofuran-3-carboxylic acid amides of general formula (Ic) have the R-configuration.

20. The method according to claim 18, wherein the amino-tetrahydrofuran carboxylic acid amide moiety of the compound of the general formula (VI) and of the 3-amino-tetrahydrofuran-3-carboxylic acid amides of general formula (Ic) have the S-configuration.

21. A method for preparing a compound of the formula (V) in high optical purity at the carbon in position 3 of the tetrahydrofuran ring 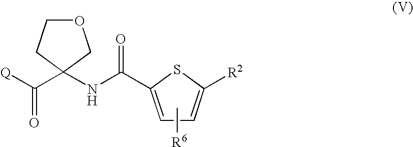
by enzymatic resolution of a racemic mixture of said compound of the formula (V),
wherein R2 and R6 are as defined in claim 1, and
wherein Q is a straight or substituted C₁₋₁₂-alkyloxy group, or allyloxy or substituted allyloxy group, or C₁₋₁₂-alkyloxycarbonyloxy or acyloxy group.

22. A compound of the formula (V) in high optical purity at the carbon in position 3 of the tetrahydrofuran ring, 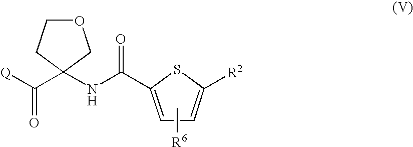
wherein R2 and R6 are as defined in claim 1, and
wherein Q is a hydroxy or C₁₋₁₂-alkyloxy group, or allyloxy or substituted allyloxy group, or a halogen atom or a C₁₋₁₂-alkyloxycarbonyloxy or acyloxy group.

23. The compound of the formula (V) in high optical purity at the carbon in position 3 of the tetrahydrofuran ring according to claim 22, wherein the amino-tetrahydrofuran carboxylic acid amide moiety of said compound of the general formula (V) has the S-configuration.

24. A method for preparing a compound of the formula (VI) in high optical purity at the carbon in position 3 of the tetrahydrofuran ring 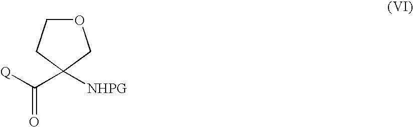
by enzymatic resolution of a racemic mixture of said compound of the formula (VI), wherein Q is a hydroxy or C₁₋₁₂-alkyloxy group, or a allyloxy or substituted allyloxy group, a halogen atom or a C₁₋₁₂-alkyloxycarbonyloxy or acyloxy group, and PG is a hydrogen atom or a protective group for the amino function.

25. A compound of the formula (VI) in high optical purity at the carbon in position 3 of the tetrahydrofuran ring, 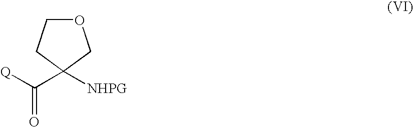
wherein Q is a hydroxy or straight or substituted C₁₋₁₂-alkyloxy group, a allyloxy or substituted allyloxy group, a halogen atom or a C₁₋₁₂-alkyloxycarbonyloxy or acyloxy group, and PG is a hydrogen atom or a protective group for the amino function.

26. The compound of the formula (VI) in high optical purity at the carbon in position 3 of the tetrahydrofuran ring according to claim 25, wherein the amino-tetrahydrofuran carboxylic acid amide moiety of said compound of the general formula (VI) has the S-configuration.

27. A method for preparing a compound of the formula (VII) in high optical purity at the carbon in position 3 of the tetrahydrofuran ring 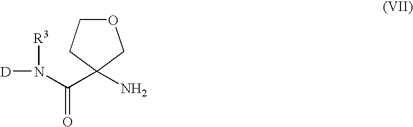
by chemical resolution of a racemic mixture of said compound of the formula (VII) with a chiral acid, wherein D and R3 are as defined in claim 1.

28. A compound of the formula (VII) in high optical purity at the carbon in position 3 of the tetrahydrofuran ring 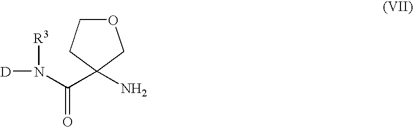
wherein D and R3 are as defined in claim 1.

29. The compound of the formula (VII) in high optical purity at the carbon in position 3 of the tetrahydrofuran ring according to claim 28, wherein the amino-tetrahydrofuran carboxylic acid amide moiety of said compound of the general formula (VII) has the S-configuration.

30. Process for preparing the compounds of general formula (XI) 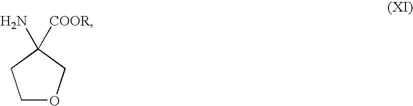
comprising reacting a ketone of the formula (X) 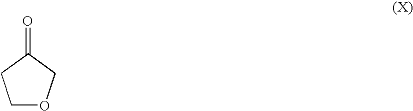
with a nitrogen source and a cyanide source in alcohol, and treating the intermediate with R—OH in the presence of an acid,
wherein R is C₁-C₁₂-alkyl, aryl, aryl-C₁-C₁₂-alkyl or a heterocycle.

31. Process according to claim 30, wherein:
a) said nitrogen source is ammonium acetate or ammonia;
b) said alcohol is methanol or ethanol;
c) said cyanide source is a cyanide salt; or
d) said acid is HCl.
